SEQUENCE LISTING (4 sequences)

SEQ ID NO: 1 - protein, 241 aa, Homo sapiens
  source 1..241: note = Pre-pro human NGF as encoded by the respective human Open Reading Frame
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 118 aa, Homo sapiens
  source 1..118: note = Mature human NGF
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 3 - protein, 118 aa, synthetic construct
  source 1..118: note = Synthetic sequence
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWEFIRIDTACVCVLSRKAVR

SEQ ID NO: 4 - protein, 118 aa, synthetic construct
  source 1..118: note = Synthetic sequence
SSSHPIFHRGEFSVCDSVSVWVGDKTTATDIKGKEVMVLGEVNINNSVFKQYFFETKCRDSNPVDSGCRGIDSKHWNSYCTTTHTFVKALTMDGKQAAWEFIRIDTACVCVLSRKAVR

(19) United States
(12) Patent Application Publication
(10) Pub. No.: US 2025/0277010 A1
(43) Pub. Date: Sep. 4, 2025

(54) AGENT FOR TREATMENT OF DERMATOLOGICAL DISORDERS

(71) Applicant: CHIESI FARMACEUTICI S.P.A., Parma (IT)

(72) Inventors: Gino VILLETTI, Parma (IT); Bruno Pietro IMBIMBO, Parma (IT); Laura CALZÀ, Parma (IT)

(21) Appl. No.: 19/093,959

(22) Filed: Mar. 28, 2025

Related U.S. Application Data
(63) Continuation of application No. 17/277,240, filed on Mar. 17, 2021, now abandoned.

(30) Foreign Application Priority Data
Sep. 17, 2018 (EP) 18194930.6
Oct. 31, 2018 (EP) 18203665.7

(51) Int. Cl. C07K 14/48; A61P 17/02

(52) U.S. Cl. CPC C07K 14/48; A61P 17/02

(57) ABSTRACT

The present invention provides a non-natural polypeptide for the treatment or prevention of skin disorders in a mammal. Administration of the polypeptide is well tolerated by the mammal. The non-natural polypeptide is provided at high purity.

### INTRODUCTION

### Field of the Invention

The present invention provides an agent suitable for the treatment and prevention of dermatological disorders, including but not limited to skin ulcers.

### Background of the Invention

Skin disorders including chronic wounds, such as ulcers, include a sore on the skin or a mucous membrane, often accompanied by disintegration of tissue. Typically, such skin disorders can result in loss of the epidermis and often portions of the dermis and even subcutaneous fat. Such skin disorders can arise can be caused by a wide variety of factors, for example but not limited to impaired blood circulation. Skin ulcers are frequent in humans, including subjects with diabetes (Ndip et al., 2012, Int. J. Gen. Med., vol. 5, p. 129-134). Such skin disorders represent a serious medical and societal problem.

At present, no suitable curative treatment for dermatological disorders of this type is available. According to current estimates, patients must be treated for months and partly for years, which results in significant costs and a high burden on patients and society (Buchberger et al., 2010, GMS Health Technol. Assess., vol. 1(6), doc. 12). In some cases, alternative measures, such as relief of pressure through the use of offloading casting devices is a principal option for management of such disorders (Ndip et al., 2012, Int. J. Gen. Med., vol. 5, p. 129-134), but such alternative measures do not provide a curative treatment at all.

Skin disorders occur often in diabetic subjects: diabetes is frequent in modern societies, and it has been estimated that one in every four patients with diabetes will develop a skin disorder, in particular a foot ulcer, during their lifetime (Ndip et al., 2012, Int. J. Gen. Med., vol. 5, p. 129-134). This disorder often requires long hospital stays, rehabilitation, home care and social services Thus, ulcers as a result of diabetes are a serious problem with an enormous impact on the overall global disease burden in view of the increasing prevalence of diabetes, and the lack of curative treatment options at present is a disadvantage for the subjects concerned, as well as for the society.

In the search for a curative treatment, in the past, some human growth factors have been proposed for therapy and potential cure of skin disorders, but it is now accepted that there is limited evidence supporting the use of human growth factors in the treatment of skin ulcers (Ndip et al., 2012, Int. J. Gen. Med., vol. 5, p. 129-134). One example of a growth factor that has been envisaged in the past for treating skin disorders is platelet derived growth factor (beclapermin, brand name Regranex), but its administration was found to be associated with severe side effects including malignancy (Buchberger et al., 2010, GMS Health Technol. Assess., vol. 1(6), doc. 12; https://www.rxlist.com/regranex-side-effects-drug-center.htm#professional). Another example tested is Granulocyte colony-stimulating factor (G-CSF), but it was found that G-CSF did not significantly affect the likelihood of resolution of infection or wound healing (Cruciani et al., 2005, Diabetes Care, vol. 28, p. 454-460). A further example proposed in the literature is Epidermal Growth Factor (EGF), which was initially proposed to have unexpected positive healing effects (e.g. WO/2003/075949 A1), but no EGF-based treatment is actually commercially available, suggesting that the initial hopes in this agent did not find support or confirmation. Alternatively, human nerve growth factor (hNGF), which has been proposed to have pro-angiogenic properties and to facilitate wound repair (Graiani et al., 2004, Diabetologia, vol. 47, p. 1047-1054) had been proposed for the treatment of certain neurophatic clinical conditions, but the clinical testing was discouraging (Apfel et al., 2000, J. Amer. Med. Assoc., vol. 284, p. 2215-2221) and, as a result, no NGF-based medicament was developed (see e.g. https://www.gene.com/media/press-releases/4875/1999-04-08/phase-iii-trial-with-nerve-growth-factor). For example, although Graiani et al. do not specifically comment on the allogenic effect of NGF, it is generally known that human NGF induces pain (Dyck et al. 1997, Neurology, vol. 48, p. 501-505; Svensson et al., 2003, Pain, vol. 104, p. 241-247). As a result, human NGF could not be established as a suitable therapeutic agent for the treatment of skin ulcers; inter alia due to its pain causing activity and/or lack of efficiency at tolerated doses. Consequently, considering that treatments based on growth factors have had limited success or proof, inter alia because of undesired side effects, the medical society has investigated other potential drugs. Based on the above, interleukins and other non-growth factor molecules have been proposed more recently for the treatment of certain skin ulcers. For example, it was proposed by Genentech that derivatives of interleukins, in particular interleukin 22, which has a proposed role in modulating the immune system, could be suitable for treating or preventing skin ulcers, including diabetic skin ulcers (see e.g. https://www.gene.com/stories/mechanisms-of-healing), but no such medicament is available to patients, and it is presently certain if this might eventually change.

Thus, there is still a need for an effective treatment of skin conditions which is not subject to adverse effects, such as intolerable or otherwise undesirable side effects, and for a therapeutic agent suitable for such purposes and available to practitioners at reliable and acceptable purity for administration to mammalian subjects, including humans.

### Problem to be Solved

It is a primary object of the invention to provide a treatment or prevention for dermatological disorders including ulcers, in diabetic and non-diabetic subjects, which is not associated with undesired or painful side effects. It is also desired to provide a therapeutically active agent at sufficient yield and purity in order to enable such treatment. Thus, an object of the present invention includes eliminating the disadvantages associated with the state of the art. Particular objects comprise the provision of a reliable method for treating s subject with a dermatological disorder without undesired side effects.

### SUMMARY OF THE INVENTION

The present invention provides a polypeptide for use in the treatment and/or prevention of a dermatological disorder in a mammalian subject, wherein the polypeptide is selected among the polypeptide of SEQ ID NO: 3 and the polypeptide of SEQ ID NO: 4. These polypeptides are characterized by a mutation of the amino acid sequence of human NGF (SEQ ID NO: 2), wherein said mutation is associated with reduced nociceptive activity. In particular, arginine at position 100 of hNGF is substituted by glutamic acid.

A particularly preferred polypeptide is the polypeptide of SEQ ID NO: 4. Said polypeptide is characterized by at least the absence of proline at position 61, more preferably by the substitution of proline at position 61 by another amino acid. In SEQ ID NO: 4, proline at position 61 of SEQ ID NO: 3 is substituted by serine.

Preferably, the mammalian subject is a human.

Preferably, the dermatological disorder is characterized by wounded surface on at least a part of the body of the subject. Preferably, the dermatological disorder is characterized by wounded surface. More preferably, the dermatological disorder is a skin lesion, preferably characterized by at least partial ablation of the dermis, and optionally of the dermis.

Preferably, the dermatological disorder comprises at least one ulcer, preferably selected from the group consisting of diabetic ulcers, trauma ulcers, surgical ulcers, pressure ulcers, chronic ulcers, and combinations of any of these ulcers. In alternative but not mutually exclusive embodiments, the dermatological disorder comprises a burn or a mechanical injury.

Preferably, the mammal, preferably the human, suffers from diabetes mellitus or has a predisposition to suffer from diabetes mellitus. In typical embodiments the diabetes mellitus is selected among diabetes mellitus Type 1 and diabetes mellitus Type 2.

In one embodiment, the polypeptide is administered in a single administration.

In an alternative and more preferred embodiment, the polypeptide is administered repeatedly. In a particularly preferred embodiment, the polypeptide is administered repeatedly one to five times per day, preferably twice per day.

In one embodiment, the polypeptide is administered repeatedly until closure of the wounded body surface. Alternatively, the polypeptide is administered repeatedly for a period of three to 30 days, preferably seven to 14 days. Optionally, administration is discontinued after completion of said interval.

In one embodiment, the polypeptide is administered to a subject with diabetic neuropathic foot ulcer (DFU), preferably onto the foot of the subject below the ankle.

Preferably, the polypeptide is for topical administration. More preferably, the polypeptide is administered onto the wounded body surface.

Preferably, the dose of the polypeptide to be administered is determined based on the surface of the wounded body surface to be treated. Preferably, the determination is conducted at the onset of treating. In one embodiment, the dosing is adjusted for later administration(s), depending on the surface of the wounded body surface at the time point of such later administration(s). In an alternative embodiment, the dosing is not adjusted for later administration(s), so that the dose of administration depends solely on the surface of the wounded body surface to be treated at the onset of the administration (first dosing), and subsequent dosages correspond to the first dose.

In one embodiment, the dose/each dose has an amount of 0.3 to 6 μg of the polypeptide per mm² of wounded body surface being treated (0.3 to 6 μg/mm²).

In one embodiment, the polypeptide is comprised in an aqueous medium, and the aqueous medium is administered to the mammalian subject.

Preferably, the treatment and/or prevention does not cause hyperalgesia in the mammalian subject.

In one embodiment, the polypeptide of SEQ ID NO: 3 or the polypeptide of SEQ ID NO: 4 is obtainable from a biological source. This may comprise purification, i.e. separation from other molecules, including other proteins, such as host cell proteins. Optionally, the polypeptide of SEQ ID NO: 3 or the polypeptide of SEQ ID NO: 4 is obtainable in a process which comprises (re-)folding and/or chromatographic purification and/or protease digestion, and optionally adjustment to final protein concentration and/preparation of a desired formulation. In one embodiment, the polypeptide is obtainable by recombinant expression and purification, wherein the purification comprises purification on a mixed mode stationary phase.

Thus, the present invention also provides the polypeptide of SEQ ID NO: 3 and the polypeptide of SEQ ID NO: 4 from a recombinant source and purified as described herein for use in a method for treatment of the human or animal body by therapy, as described herein.

### DETAILED DISCLOSURE OF THE INVENTION

This specification in its entirety, together with the claims and the figures, discloses specific and/or preferred embodiments and variants of the individual features of the invention. The present invention also contemplates as particularly preferred embodiments those embodiments, which are generated by combining two or more of the specific and/or preferred embodiments and variants described herein for the present invention. Thus, the present disclosure also includes all of the entities, compounds, features, steps, methods or compositions referred to or indicated in this specification, individually or collectively, and any and all combinations or any two or more of said entities, compounds, features, steps, methods or compositions. Thus, unless specifically stated otherwise herein or the context requires otherwise, reference to a single entity, compound, feature, step, method or composition shall be taken to encompass one and a plurality (i.e. more than one, such as two or more, three or more or all) of those entities, compounds, features, steps, methods or compositions. Unless specifically stated otherwise or the context requires otherwise, each embodiment, aspect and example disclosed herein shall be taken to be applicable to, and combinable with, any other embodiment, aspect or example disclosed herein.

The person of ordinary skill in the art will appreciate that the invention described herein is susceptible to variations and modifications other than those specifically described. Thus, the present disclosure is not limited in scope by the specific embodiments described herein, which are provided herein for the purposes of illustration and of exemplification. Functionally or otherwise equivalent entities, compounds, features, steps, methods or compositions are within the scope of the present disclosure. it will be apparent to the person of ordinary skill in the art that the present disclosure includes all variations and modifications of the entities, compounds, features, steps, methods or compositions literally described herein.

Each of the references cited herein (including all patents, patent applications, scientific publications, manufacturer's specifications, instructions, presentations, etc.), whether above or below, are hereby incorporated by reference in their entirety. Nothing herein is to be construed as an admission that the present invention would not be entitled to antedate a specific teaching and/or as an admission that a specific reference, other than the common general knowledge, contains information sufficiently clear and complete for it to be carried out by a person skilled in the art.

Generally, unless specifically defined otherwise, all technical and scientific terms used herein have the same meaning as commonly understood by one of ordinary skill in the art (e.g., in medicine, dermatology, neurology, genetics, molecular biology, gene expression, cell biology, cell culture, immunology, neurobiology, chromatography, protein chemistry, and biochemistry). Textbooks and review articles published e.g. in English typically define the meaning as commonly understood by a person of ordinary skill in the art.

The expression “and/or”, e.g., “X and/or Y” shall be understood to mean either “X and Y” or “X or Y” and shall be taken to provide explicit disclosure of “and”, of “or” and of both meanings (“and” or “or”).

As used herein, unless specified otherwise, the terms “about”, “ca.” and “substantially” all mean approximately or nearly, and in the context of a numerical value or range set forth herein preferably designates+/−10%, more preferably +/−5%, around the numerical value or range recited or claimed.

Unless expressly specified otherwise, the word “comprise”, or variations such as “comprises” or “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”.

Unless expressly specified otherwise, all indications of relative amounts regarding the present invention are made on a weight/weight basis. Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

All methods and processes described herein can be performed in any suitable order unless otherwise indicated herein or unless the context clearly dictates otherwise.

The term “agent” as used herein, unless specified otherwise, generally refers to a compound or composition, preferably to a compound. An agent is capable of producing an effect on a living organism and/or on a cell from a living organism or derived from a living organism, e.g. by acting on a cell and/or on body tissue, or in an environment. The physical state of an agent is not particularly limited and, unless specified otherwise, may be in the air, water, and/or solid state. The type of agent is not particularly limited, unless specified otherwise, and thus, an agent may be a chemical and/or a biomolecule such as a protein or a nucleic acid. Specific agents defined herein are useful in the present invention.

An “adverse effect”, as used herein, is an undesired harmful effect resulting from an administration of an agent (a drug) to a subject. Adverse effects include, without limitation, morbidity, mortality, hyperalgesic syndrome, pain, alteration in body weight, levels of enzymes, loss of function, or any pathological change detected at the microscopic, macroscopic or physiological level. Adverse effects may cause a reversible or irreversible change, including an increase or decrease in the susceptibility of the individual to other chemicals, foods, or procedures, such as drug interactions.

As used herein, the terms “chromatography”, “chromatographic” and the like generally refer to a technique suitable for the separation of a mixture, wherein the mixture is added to a non-liquid material called the “stationary phase” with the purpose to separate, at least partially, one or more constituents of the mixture. For that purpose, the stationary phase may be exposed to a fluid and/or the mixture may be dissolved in a fluid; said fluid contacted with the stationary phase may also referred to as “mobile phase”. In general, any step that is “carried out by chromatography”, as described herein, may synonymously referred to as a “chromatographic step”.

The term “mobile phase” as used herein, has the meaning typically used in the art and can refer to all fluids brought in contact with the stationary phase during chromatography, i.e. to wash fluids as well as to fluids (mixtures) comprising a protein of interest, such as one or more of the proteins described herein. In the present invention, the mixture subjected to chromatography, as specified herein, typically comprises one or more proteins, such as in particular the proteins described herein, such as the polypeptides of SEQ ID NO: 3 or 4, a precursor of any of these, a protease, and/or host cell proteins (HCP).

A “stationary phase” typically comprises a typically comprises a base matrix, which is a water-insoluble material, usually in particle from or gel form, such as a resin. In many cases, including embodiments described herein, a stationary phase comprises a base matrix and a moiety that can bind to at least one component comprised in the mixture that is to be subjected to chromatography. The base matrix is normally a water-insoluble material, usually in particle from or gel form. Non-limiting examples of base matrices are sepharose and agarose, for example highly rigid agarose.

A “chromatographic step” as used herein, refers to the action of adding to a chromatography material (preferably a stationary phase) a liquid comprising at least one compound to be analyzed and/or to be purified, which is preferably a protein (and in the context of the present invention said protein is most preferably the polypeptide of SEQ ID NO: 3 or SEQ ID NO: 4), optionally washing the chromatography material with one or more wash solutions, and eluting said at least one compound. In that context, a process characterized by two chromatographic steps, for illustration, is characterized in that a liquid comprising at least one such compound to be analyzed and/or to be purified is added to a first chromatographic material, as above described, and, after elution therefrom, the liquid comprising at least one such compound is added to a second chromatographic material, from which it is also eluted, as' above described. It is the aim of any “chromatographic step” that at least one component comprised in the mixture applied to a stationary phase, preferably in chromatography, binds to the stationary phase. Such compound may be one or more proteins described herein. The compound may be recovered from the stationary phase, e.g. by exchange of mobile phase and/or by continued exposure to the mobile phase overtime.

The term “binds”, when used with reference to chromatography, such as to describe the binding capacity of a stationary phase, is not particularly limited, but typically refers to non-covalent binding. Thus typically at least one component comprised in a mixture, such as at least one protein, binds non-covalently to the stationary phase. A chromatographic step optionally but preferably comprises the washing of the stationary phase to which the at least one component is bound. The at least one component may be at least one protein, such as at least one protein described herein.

The term “heterologous” as used herein describes something consisting of multiple different elements.

The terms “disulfide” and “disulfide bond” are used, in the context of the present invention, within the meaning commonly used in the art. In general, a “disulfide” refers to a functional group with the structure R—S—S—R′. The linkage is also called an “SS-bond” and is usually derived by the coupling of two thiol groups. Disulfide bonds in proteins are formed between the thiol groups of the cysteine residues by the process of oxidative folding; such a specific disulfide bond between the thiol groups of two cysteine residues can also be referred to as “disulfide bridge” Without wishing to be bound to a particular theory, it is normally understood in the art that, in In eukaryotic cells, disulfide bridges are formed in the lumen of the endoplasmic reticulum (and the mitochondrial intermembrane space) but not generally in the cytosol, and, regarding prokaryotes, disulfide bridges are formed in the periplasm (of respective organisms, particularly Gram-negative bacteria); disulfide bridges can also be found in proteins of the extracellular environment of both eukaryotic and prokaryotic cells.

The terms “express”, “expressed” and “expression”, “gene expression” and the like, as used herein, relate to the use of information from a gene in the synthesis of a functional gene product. Gene expression comprises at least the transcription, and optionally comprises one of more additional features, optionally selected from the open list comprising translation and post-translational modification. In the context of recombinant expression of a protein in a host cell, the term normally implies that the protein is produced by the host cell (in any compartment of the cell and/or secreted and/or incorporated in inclusion bodies), unless the context dictates otherwise.

The term “heterologous” as used herein describes something consisting of multiple different elements or origins. For example, in a non-human host cell which comprises a human gene (or gene encoding a non-natural polypeptide, such as the polypeptide of the invention) said gene is “heterologous” to the cell, and the cell may be capable of “heterologous” expression of the respective gene. Heterologous gene expression can also be referred to as “recombinant”.

The term “inclusion body” has the meaning typically used in the art and is meant to refer to aggregates or particles found in the cytosol or in the periplasm of a host cell; inclusion bodies typically comprise protein, such as, in particular, protein expressed recombinantly in the host cell. Without wishing to be bound to any particular theory, it is understood that in the field of recombinant expression, inclusion bodies typically contain the recombinantly expressed protein but relatively little host cell protein (HCP), ribosomal components or DNA/RNA fragments. Without wishing to be bound to any particular theory, it is understood that inclusion bodies typically comprise, at least in part, protein that is not properly folded (misfolded protein), in particular misfolded recombinantly expressed protein. It is understood that inclusion bodies typically comprise protein in a non-properly folded form, i.e. in the context of the present invention they typically comprise the polypeptide according to the present invention and/or a precursor thereof, in a non-properly folded form. The term “misfolded” generally describes a biomolecule, such as a nucleic acid or polypeptide, which is not on the native conformation, i.e. in a non-properly folded form

By “isolated” is meant material that is substantially or essentially free from components that normally accompany it in its native state. For example, an “isolated peptide” or “isolated protein”, as used herein, refers to a peptide or protein, respectively, which has been purified from the cellular and extracellular environment, such as tissue, which surround it in a naturally-occurring state, e.g., from the cell in which it has been expressed, such as a host cell. In an alternative description, an “isolated peptide” or “isolated protein” and the like, as used herein, refer to in vitro isolation and/or purification of a peptide or protein, respectively, from its natural cellular environment, and from association with other components of the environment in which the peptide or protein normally resides. In another example, an “isolated cell”, as used herein, refers to a cell, which has been purified from the cellular and extracellular environment, such as tissue or cell colonies, which surround it in a naturally-occurring state, e.g., a host cell which has been removed from the environment that is normally adjacent to the cell. In accordance with the above definition of the word “isolated”, “to isolate”, as used herein, is the verb that describes the activity to obtain “isolated” material, such as e.g. an isolated cell or an isolated peptide or protein.

The terms “multi” and “multiple” as used herein mean a multitude, i.e. any number of two or more.

The term “mutation”, as used herein, refers to the alteration of the nucleotide sequence of the genome of an organism, virus, or extrachromosomal DNA or other genetic elements. The term also extends to mutations of an amino acid sequence, particularly the amino acid sequence of a gene that carries at least one (non-silent) mutation. Unless specified otherwise, a mutation of the nucleotide sequence is a permanent alteration. Mutations present in the germ line are normally inheritable. In general, a mutation of the nucleotide sequence can result in many different types of change in sequences: mutations in genes can either have no effect, alter the product of a gene, or prevent the gene from functioning properly or completely. Mutations can also be present in non-genic regions. Unless specified otherwise, the wild type sequence is used as a reference sequence to describe a mutation. Thus, for example, when it is said that a given mutant is characterized by mutation of position 100 of a polypeptide sequence, this indicates that at position 100 the mutant does not have the same amino acid residue as the wild type polypeptide. Specific types of mutations of a nucleotide sequence and/or an amino acid sequence include alterations such as deletions, substitutions, additions, insertions and splice variants. A “deletion” with respect to a nucleotide sequence refers to the absence of one or more nucleotide(s) in the nucleotide sequence. A “deletion” with respect to an amino acid sequence refers to the absence of one or more amino acid residue(s) in the polypeptide. An “addition” with respect to a nucleotide sequence refers to the presence of one or more additional nucleotide(s) in nucleotide sequence. An “addition” with respect to an amino acid sequence refers to the presence of one or more additional amino acid residue(s) in the related polypeptide. A “substitution” with respect to a nucleotide sequence refers to the replacement of one or more nucleotide(s) by (an) other nucleotide(s) in the nucleotide sequence. A “substitution” with respect to an amino acid sequence refers to the replacement of one or more amino acid residue(s) by (an) other amino acid residue(s) in the polypeptide. Additions, deletions and substitutions to a nucleotide sequence, such as to an open reading frame, may be 5′ terminus, the 3′ terminus, and/or internal. Additions, deletions and substitutions to a polypeptide, may be at the amino terminus, the carboxy terminus, and/or internal. An “insertion” with respect to a nucleotide sequence and/or a polypeptide sequence is an addition of one or more nucleotides, or one or more amino acid residues, respectively, specifically at an internal position of the respective sequence. The term “splice variant” is used to describe that the RNA encoding a polypeptide sequence is spliced differently from the respective wild type RNA, typically as a result of a mutation at nucleic acid level, usually resulting in a polypeptide translation product which is different from the wild type polypeptide. The term “splice variant” can be used not only with respect to the respective RNA, but also with respect to the respective template DNA sequence (typically genomic DNA) and with respect to the sequence of the polypeptide encoded by such RNA.

The term “mutant” is generally intended to refer to a nucleic acid sequence or amino acid sequence which is different from the wild type sequence. A mutant nucleic acid sequence or amino acid sequence thus has at least one mutation with respect to the respective wild type sequence. In cases where polymorphisms at the nucleic acid sequence exist which are, however, not reflected at the level of the respective encoded polypeptide (silent mutations, degeneracy of the genetic code), the term “mutant”, on nucleic acid level, specifically refers only to those nucleic acid variants which encode a mutant polypeptide. Mutants can contain different combinations of mutations, alone or in combination, including more than one mutation and different types of mutations.

The term “nerve growth factor”, abbreviated “NGF” or “beta-NGF” stands for a neurotrophic factor and neuropeptide involved in the regulation of growth, maintenance, proliferation, and survival of certain neurons and other cells, in accordance with the common meaning in the art (see e.g. Levi-Montalcini, 2004, Progress in Brain Research, vol. 146, p. 525-527). Unless the context dictates otherwise, the term nerve growth factor stand for wild-type NGF only and does not include the polypeptides of SEQ ID NO: 3 or 4. Wild-type NGF is the 2.55, 26-kDa beta subunit obtainable form a NGF precursor, which is biologically active: wild-type NGF binds with at least two classes of receptors: the tropomyosine receptor kinase A (TrkA) and low-affinity NGF receptor (LNGFR/p75NTR). The term “NGF”, unless specified otherwise, refers to NGF of any species, preferably mammalian species; however, human NGF is always preferred, “hNGF”, as used herein stands for human NGF. Unless the context dictates otherwise, the terms “NGF” and “hNGF” refer to wild-type NGF, i.e. hNGF stands for wild-type NGF. The amino acid sequence of wild-type human NGF corresponds to positions 121-239 of SEQ ID NO: 1 (grey in FIG. 24). The sequences of non-human NGF are available, e.g., in the scientific literature, through sequence searches, such as BLAST, using positions 121-239 of SEQ ID NO: 1 as bait, and in public protein databases such as Swissprot.

The terms “NGF mutein” and “mutein of NGF”, or, with reference to NGF “mutein thereof”, are used herein interchangeably to refer to a polypeptide which is characterized by at least one mutation, compared to wild-type NGF, as further described in detail herein. The polypeptides of SEQ ID NO: 3 and SEQ ID NO: 4 are muteins of NGF. Preferably a mutein of NGF has 80 to 99.5% sequence identity with NGF, particularly human NGF, more preferably a mutein has 90 to 99% sequence identity with NGF, particularly human NGF.

The terms “mature part” “mature portion”, with reference to NGF, are used interchangeably with the term “beta-NGF” and refer to a polypeptide of NGF which is characterized in that it does not comprise the pro-peptide (and hence, of course, not the pre-pro-peptide) of NGF. In analogy, the term “mature part” is also used to refer to the polypeptides of SEQ ID NO: 3 or 4, as these polypeptides likewise do not comprise a pro-peptide (and hence, of course, not a pre-pro-peptide). Preferably, the mature part does also not comprise a C-terminal cleavable peptide encoded by the wild-type NGF open reading frame; such C-terminal cleavable peptide, in the case of human NGF, consists of the two amino acid residues “RA” (240 and 241 in SEQ ID NO: 1). More particularly, the mature part is obtainable, without limitation, by cleavage of a pro-NGF with the protease Furin (and with other proteases capable of precisely cleaving directly N-terminal of the first amino acid residue of NGF, or of the polypeptide of SEQ ID NO: 3 or 4, respectively. For example, the Furin cleavage site of human NGF, and of many orthologs, is well known to consist of the sequence R¹S²K³R⁴ (one letter amino acid code, sequences numbered from N to C terminus; boxed in FIG. 25)). In mature NGF, normally neither the Furin cleavage site nor any amino acid N-terminally of the Furin cleavage site is present. For illustration, the mature part of human NGF consists of the polypeptide represented by amino acid positions 122-239 of SEQ ID NO: 1. The mature part of non-human NGF may be identified, e.g. by sequence search and/or sequence analysis, wherein said mature part of human NGF is used for sequence alignment.

The term “precursor”, as used herein with reference to NGF, refers to any peptide sequence from which NGF is obtainable through proteolytic cleavage. For illustration, both pro-NGF and pre-pro-NGF, as well as variants thereof, are typical examples of precursors of NGF. The term “precursor” as used herein, can refer to precursors the most C-terminal amino acid residue of which is the most C-terminal residue of NGF, and also to precursors which extend at the C-terminus beyond the most C-terminal residue of NGF, as long as NGF is obtainable therefrom by proteolytic cleavage: although the naturally occurring precursor of wild-type human pro-NGF (SEQ ID NO: 1) comprises a C-terminal dipeptide (amino acid residues 240 and 241 in SEQ ID NO: 1, bold in FIG. 1), it is preferable in the present invention that the precursor does not comprise a C-terminal cleavable peptide encoded by the wild-type NGF open reading frame; such C-terminal cleavable peptide, in the case of human NGF, consists of the two amino acid residues “RA” (240 and 241 in SEQ ID NO: 1.

The terms “pre-peptide” or “pre-sequence”, as used herein, generally interchangeably refer to a polypeptide sequence encoded by part of the NGF open reading frame, N-terminally directly adjacent to the pro-peptide. For illustration: a pre-peptide is NGF consists of the sequence comprising the continuous sequence ranging from reside 1 of SEQ ID NO: 1 to residue 18 of SEQ ID NO: 1. The sequences of the respective pre-peptides of precursors of non-human NGF are available, e.g., in the scientific literature, through sequence searches, such as BLAST, using positions 1-18 of SEQ ID NO: 1 as bait, and in public protein databases such as Swissprot. A polypeptide or protein consisting of the pre-peptide and of pro-NGF, wherein the C-terminus of the pre-peptide is directly adjacent to the N-terminus of pro-NGF, can be referred to herein as “pre-pro-NGF”.

The terms “pro-peptide” or “pro-sequence”, as used herein, generally interchangeably refer to a polypeptide sequence encoded in nature by part of the NGF open reading frame, N-terminally directly adjacent to mature NGF, but which polypeptide sequence does not include the pre-peptide. For illustration: a pro-peptide is comprised in the wild-type precursor of NGF. The pro-peptide of the precursor of NGF, consists of the sequence comprising the continuous sequence ranging from residue 19 of SEQ ID NO: 1 to residue 121 of SEQ ID NO: 1. The sequences of the respective pro-peptides of non-human pro-NGF are available, e.g., in the scientific literature, through sequence searches, such as BLAST, using positions 19-121 of SEQ ID NO: 1 as bait, and in public protein databases such as Swissprot.

“pro-NGF”, as used herein, refers to a peptide sequence comprising both the mature part of NGF and the respective pro-peptide, but not the respective pre-peptide. Human pro-NGF consists of the sequence comprising the continuous sequence ranging from reside 19 of SEQ ID NO: 1 to at least residue 239 of SEQ ID NO: 1. Although wild-type human pro-NGF comprises a C-terminal dipeptide (amino acid residues 240 and 241 in SEQ ID NO: 1, bold in FIG. 25), it is preferable that the pro-NGF obtained and used in the present invention does not comprise a C-terminal cleavable peptide encoded by the wild-type NGF open reading frame; such C-terminal cleavable peptide, in the case of human NGF, consists of the two amino acid residues “RA” (240 and 241 in SEQ ID NO: 1). The sequences of non-human pro-NGF are available, e.g., in the scientific literature, through sequence searches, such as BLAST, using positions 19-239 of SEQ ID NO: 1 as bait, and in public protein databases such as Swissprot.

The terms “nucleic acid” and “polynucleotide” are used interchangeably herein, and refer to both RNA and DNA, including cDNA, genomic DNA, synthetic DNA, and DNA/RNA equivalents containing nucleotide analogs, phosphate analogs and/or sugar analogs. A nucleic acid can be double-stranded or single-stranded (i.e., a sense strand or an antisense strand). Non-limiting examples of polynucleotides include genes, open reading frames, gene fragments, exons; introns, messenger RNA (mRNA), transfer RNA, ribosomal RNA, siRNA, micro-RNA, ribozymes, cDNA, recombinant polynucleotides, branched polynucleotides, plasmids, vectors, isolated nucleic acids of any type and sequence nucleic acid probes, and primers, as well as nucleic acid analogs. Nucleic acids may have any type of three-dimensional structure.

The term “peptide” according to the invention comprises oligo- and polypeptides and refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently to a chain by peptide bonds.

The term “protein” preferably refers to large peptides, preferably to peptides with more than 100 amino acid residues, but in general the terms “peptide”, “polypeptide” and “protein” are synonyms and are used interchangeably herein, unless the context dictates otherwise. Thus, the terms. “polypeptide of SEQ ID NO: 4 and “protein of SEQ ID NO: 4” have the identical meaning.

The term “pharmaceutically acceptable” generally describes that a certain substance can be administered to a subject, optionally and preferably in combination with an agent, without the agent causing intolerable adverse effects, at the dosage used.

The terms “pharmaceutically acceptable carrier” and “pharmaceutically acceptable excipient” are used to refer to any one or more of solvents, dispersion media, coatings, antibacterial and antifungal agents, isotonic and absorption delaying agents, and the like that are physiologically compatible and are suitable for administration to a subject as described herein, or do not otherwise interfere with such administration. Examples of such pharmaceutically acceptable carriers comprise without limitation one or more of water, saline, phosphate buffered saline, dextrose, glycerol, ethanol and the like, as well as combinations thereof. Particularly for the case of liquid pharmaceutical compositions, it may be preferable to include isotonic agents, for example, sugars, polyalcohols such as mannitol, sorbitol, or sodium chloride in the composition. Pharmaceutically acceptable carriers may further comprise auxiliary substances such as wetting or emulsifying agents, preservatives or buffers, which enhance the shelf life or effectiveness of the agent. A pharmaceutically acceptable carrier is typically comprised in a composition according to the present invention.

The term “pharmaceutically active agent” refers to an agent that can be used in the administration to a subject where the agent would be of benefit, e.g., in ameliorating the symptoms of a disease or disorder. In addition, a “pharmaceutically active agent” can have a positive or advantageous effect on the condition or disease state of a subject when administered to the subject in a therapeutically effective amount. Preferably, a pharmaceutically active agent has curative properties and may be administered to ameliorate, relieve, alleviate, reverse, delay onset of or lessen the severity of one or more symptoms of a disease or disorder. A pharmaceutically active agent may have prophylactic properties and may be used to delay the onset of a disease or to lessen the severity of such disease or pathological condition. For example, an agent of the invention is considered herein as a pharmaceutically active ingredient for the treatment of cystic fibrosis, as claimed. In another example, a pharmaceutically active protein can be used to treat a cell or an individual which does not normally express a protein, or not at the desired levels, or which mis-expresses a protein, e.g., a pharmaceutically active protein can compensate for a mutation, or for lack of sufficiently high expression, by supplying a desirable protein. The term “pharmaceutically active peptide or protein” includes entire proteins or polypeptides, and can also refer to pharmaceutically active fragments thereof. It can also include pharmaceutically active analogs of a peptide or protein.

An “open reading frame” or “ORF” is a continuous stretch of codons beginning with a start codon and ending with a stop codon.

The terms “subject” and “patient”, as used herein, relate to a mammal. For example, mammals in the context of the present invention are humans, non-human primates, domesticated animals including but not limited to dogs, cats, sheep, cattle, goats, pigs, horses etc., laboratory animals including but not limited to mice, rats, rabbits, etc., as well as animals in captivity such as animals of zoos. The terms “subject” and “patient” as used herein particularly include humans. The subject (human or animal) has two sets of chromosomes; that is, the subject is diploid. The term “patient” refers to a subject which suffers from a condition, is at risk of suffering from a condition, has suffered from a condition, or is predicted to suffer from a condition, and which may be subjected to therapy, e.g. by administration of an agent. The patient's condition may be chronic and/or acute. Thus, a “patient” can also be described as a subject subjected to a therapy and/or or in need of a therapy.

The term “therapy” is to be understood broadly and refers to the treatment of a subject with the goal to prevent or treat a condition in the subject. In preferred embodiments, therapy specifically includes the administration of an agent to the subject.

The term “trypsin”, as used herein, generally refers to a proteolytic enzyme classified as EC 3.4.21.4). Trypsin cleaves peptide chains mainly at the carboxyl side of the amino acids lysine or arginine, normally except when either is followed by proline. Without wishing to be bound by theory, it is understood that trypsin is a serine protease, and that trypsin is naturally found in the digestive system of many vertebrates, where it hydrolyzes proteins. Preferred in the present invention is trypsin from recombinant sources. Although, in vivo, trypsin is formed together with a pro-peptide (termed “trypsinogen”), the term “trypsin”, as used herein preferably refers to mature trypsin devoid of any pro-peptide. The use of trypsin for proteolytic cleavage can also be referred to as “trypsin proteolysis” or “trypsinization”, and proteins that result from cleavage with trypsin are said to have been “trypsinized”.

A “variant” of a precursor of NGF or of the polypeptide of SEQ ID NO: 3 or 4, refers to a polypeptide or protein wherein the amino acid sequence that is not part of the mature NGF (beta-NGF) or not part of SEQ ID NO: 3 or 4, respectively, is characterized by at least one mutation in comparison with a wild-type precursor of NGF, such as with a wild-type pro-NGF or a wild-type pre-pro-NGF; said at least one mutation is preferably found N-terminal to the amino acid sequence of the mature NGF (beta-NGF). Thus, as used herein, a “variant” of a precursor of NGF or the like refers to a peptide or protein wherein the pre-peptide and/or the pro-peptide is characterized by at least one mutation, with respect to the amino acid sequence of the pre-peptide and/or the pro-peptide, for example but without limitation those variants described in WO 2013/092776 A1 and in by US 2018/0086805 A1. For illustration, WO 2013/092776 A1 describes “variants” of pro-NGF wherein the (wild-type) Furin cleavage site is absent due to one or more specific mutations.

The term “vector” or “cloning vector” generally refers to a nucleic acid that can be introduced into a host cell. Example vectors include, without limitation, plasmids, phages and all other types of nucleic acids that can be introduced into a host cell. The term “vector” is to be understood broadly and will comprise vectors which encode a peptide or protein for heterologous expression (such vectors may serve as templates, for the generation of transcripts), and those which do not. Vectors of the first type will contain an open reading frame encoding a protein or peptide, which may be expressed, when the vector is present in a host cell. Although the type of vector that the skilled person will choose will be dependent on the type of host cell that the skilled person will choose, in a particular case, cloning vectors for all common host cells, including E. coli, are commercially available, and the skilled person will thus choose a particular vector in full consideration of the host cell chosen.

The term “wild type” is used herein to refer to a gene or a protein typically found in nature, preferably in a healthy subject. A gene or a protein that is not “wild type” is referred to herein as “mutant” or “mutated”, or the like. For illustration, SEQ ID NO: 1 shows the amino acid sequence of a precursor of wild-type human NGF; SEQ ID NO: 2 shows the amino acid sequence of wild-type human NGF.

The present invention is based on several findings, which are interrelated and thus together lead the inventors to arrive at the various aspects of the invention, which will all be described individually in the following.

### The Agent According to the Present Invention

The present invention provides an agent for the treatment and/or prevention of dermatological disorder in a mammalian subject. The agent that can be used in the administration to a subject where the agent would be of benefit, e.g., in ameliorating the symptoms of a disease or disorder. IN particular, the agent useful in the present invention is a polypeptide of SEQ ID NO: 3 or SEQ ID NO: 4. Thus, the present invention, in particular, provides a polypeptide of SEQ ID NO: 3 or SEQ ID NO: 4 for use in therapy. The therapy typically comprises administration of said polypeptide to a human or animal body, as described herein below.

According to the present invention the polypeptide of SEQ ID NO: 3 and SEQ ID NO: 4 are provided as pharmaceutically active agents. By the present invention the polypeptide of SEQ ID NO: 3 or SEQ ID NO: 4 is provided for medical use, in particular for the treatment and/or prevention of dermatological disorder in a mammalian subject. Optionally, the polypeptide of SEQ ID NO: 3 or SEQ ID NO: 4 is from a recombinant source. Thus, the present invention provides also the recombinant polypeptide of SEQ ID NO: 3 or SEQ ID NO: 4 for medical use, as described herein.

The agent according to the present invention, also termed herein “polypeptide of SEQ ID NO: 3” or “polypeptide of SEQ ID NO: 4” will now be described in more detail. The term “polypeptide of SEQ ID NO: 3” and similar terms denote herein a polypeptide comprising the amino acid sequence defined by SEQ ID NO: 3 and/or an agent with equivalent biological activity. The term “polypeptide of SEQ ID NO: 4” and similar terms denote herein a polypeptide comprising the amino acid sequence defined by SEQ ID NO: 4 and/or an agent with equivalent biological activity. Thus, within these terms term are also included functionally equivalent parts or analogues of such polypeptides. One example of a biologically equivalent part of the polypeptide could be a domain or subsequence of the polypeptide of SEQ ID NO: 3 or the polypeptide of SEQ ID NO: 4, which includes the binding site to enable the domain or subsequence to exert substantially the same biological activity as the full-length polypeptide of SEQ ID NO: 3 or the full-length polypeptide of SEQ ID NO: 4 or alternatively a gene coding for such a polypeptide. The term “substantially the same biological activity” refers to an equivalent part or analogues polypeptide having at least 50%, preferably at least 60%, more preferably at least 70%, more preferably at least 75%, more preferably at least 80%, more preferably at least 85%, more preferably at least 90%, more preferably at least 95% and most preferably at least 97%, at least 98% or at least 99% of the activity of the polypeptide of SEQ ID NO: 3 or the polypeptide of SEQ ID NO: 4 in the assays described in Examples 3 and 4. An example of a biologically equivalent analogue of the polypeptide could be a fusion protein which includes at least a part of the amino acid sequence of the polypeptide of SEQ ID NO: 3 or the polypeptide of SEQ ID NO: 4, but it can also be a homologous analogue of the polypeptide. Also, completely synthetic molecules that mimic the specific biological activity of the polypeptide of SEQ ID NO: 3 or the polypeptide of SEQ ID NO: 4 would constitute “biologically equivalent analogues”.

More preferably, the term “polypeptide of SEQ ID NO: 3” and similar terms denote herein a polypeptide comprising the amino acid sequence defined by SEQ ID NO: 3; such agents are optionally fusion proteins which comprise inter alia the amino acid sequence defined by SEQ ID NO: 3. Most preferably, the term “polypeptide of SEQ ID NO: 3” and similar terms denote herein a polypeptide consisting of the amino acid sequence defined by SEQ ID NO: 3; in this embodiment, the agent consists of a polypeptide consisting of the 118 amino acid residues in sequential order as defined by SEQ ID NO: 3. In this and other embodiments, the polypeptide optionally carries one or two or three internal cysteine bonds, so that cysteine (Cys, C) residues are covalently linked to each other to form intramolecular disulfide bridges. The cysteine bonds are preferably equivalent to those in-wild-type human NGF.

Equally more preferably, the term “polypeptide of SEQ ID NO: 4” and similar terms denote herein a polypeptide comprising the amino acid sequence defined by SEQ ID NO: 4; such agents are optionally fusion proteins which comprise inter alia the amino acid sequence defined by SEQ ID NO: 4. Most preferably, the term “polypeptide of SEQ ID NO: 4” and similar terms denote herein a polypeptide consisting of the amino acid sequence defined by SEQ ID NO: 4; in this embodiment, the agent consists of a polypeptide consisting of the 118 amino acid residues in sequential order as defined by SEQ ID NO: 4. In this and other embodiments, the polypeptide optionally carries one or two or three internal cysteine bonds, so that cysteine (Cys, C) residues are covalently linked to each other to form intramolecular disulfide bridges. The cysteine bonds are preferably equivalent to those in wild-type human NGF.

The polypeptide of the present invention may optionally be characterized by further posttranslational modifications. Such posttranslational modifications optionally include glycosylation and/or phosphorylation. Preferably, however, the polypeptide according to the present invention is free of glycosylation and/or phosphorylation. Indeed, considering that the experimental examples herein demonstrate a beneficial effect on the healing of skin disorders and a beneficial benefit-to-adverse effect ratio, whereby the polypeptide used was obtained by cytosolic recombinant expression in bacteria, which typically does not result in glycosylation and/or phosphorylation, it is plausible that the beneficial effect of the present invention is not dependent on such type of posttranslational modification. Therefore, in preferred embodiments, the polypeptide according to the present invention is not characterized by glycosylation and/or phosphorylation.

Typically, the polypeptide according to the present invention is a non-natural polypeptide which is not naturally produced by the subject to which the polypeptide is administered. This is associated not only with the advantage of detectability in the subject post administration, but also evidences that administration (from an external source, such as e.g. the compositions prepared according to the present disclosure) needs to be administered to the subject in order to achieve success in treatment or prevention of the disorder.

Preferably the polypeptide according to the present invention is an isolated polypeptide. More preferably, the polypeptide according to the present invention is essentially free of host cell proteins, degradation products (such as des-nona variant, for example), and protease (such as trypsin, for example). When the polypeptide according to the present invention is essentially free of host cell proteins, degradation products (such as des-nona variant, for example), and protease (such as trypsin, for example) is may also be referred to as “pure polypeptide”. Preferably, the polypeptide according to the present invention is administered as pure polypeptide. More preferably, the pure polypeptide consisting of SEQ ID NO: 3 and/or the pure polypeptide consisting of SEQ ID NO: 4 has a weight percentage of 90% or more, preferably 92% or more, more preferably 93% or more, more preferably 94% or more, more preferably 96% or more, more preferably 97% or more, more preferably 98% or more, more preferably 99% or more, more preferably 99.2% or more, more preferably 99.4% or more, more preferably 99.6% or more, more preferably 99.8% or more, more preferably 99.9% or more, with respect to the total protein in the composition. Such pure polypeptide is available based on the disclosure herein, including Examples 1 and 2. Most preferably, the pure polypeptide according to the present invention has a purity grade compatible with Good manufacturing practices (GMP).

As demonstrated in the experiments herein, particularly Examples 3 and 4, the administration of the agent according to the present invention did not induce any hyperalgesic syndrome (pain), notwithstanding the fact that the agent was put into direct contact with fully exposed nociceptive fibers (nerves); they are considered fully exposed because of the lack of skin, and they are considered to be hyperactivated as a result of the skin lesion. The absence of pain in this extreme setting is particularly remarkable because the agent was administered by topically, and repeatedly, to the damaged skin, also in a chronic setting (for details see Examples). This is also particularly remarkable in view of the discouraging earlier studies with human NGF exposed to an innervated area, i.e. an area characterized by exposed nociceptors (Svensson et al, 2003, Pain, vol. 104, p. 241-247). The above surprising findings cannot be explained solely by the fact that the agent according to the present invention has previously been described as “painless”, since its ability to induce pain has never been experimentally investigated in an innervated area, i.e. an area characterized by exposed nociceptors, let alone on hyperactivated nerves, as in the case of a skin lesion. Furthermore, even though administration of the agent according to the present invention is causative for tissue reinnervation (see e.g. Example 3), the administration is not associated with pain. In addition to that, the positive effect of the agent according to the present invention on angiogenesis (see experimental examples) is surprising and was not predictable based on the state of the art. Angiogenesis is understood to be of particular importance for tissue formation and wound closure. In summary, the combination of these advantageous effects is highly surprising in light of the state of the art.

Optionally, according to the present invention, the polypeptide of SEQ ID NO: 3 or SEQ ID NO: 4 is administered in an effective amount to a subject in need thereof. Details of the administration, the effective amount and of the subject in need thereof are described herein below.

The polypeptides consisting of SEQ ID NO: 3 and of SEQ ID NO: 4, respectively, differ in one or two positions from the amino acid sequence of human nerve growth factor (NGF, also referred to as wild-type human NGF or wild-type NGF, see SEQ ID NO: 2). The difference of the polypeptide according to the present invention with respect to the polypeptide of SEQ ID NO: 2 has a remarkable effect on the treatment or prevention of skin disorders and the absence of side effects, as disclosed in detail herein and supported by the experimental examples herein.

Nerve growth factor (NGF) is a neurotrophin required for the development and survival of specific neuronal populations. NGF is a homodimeric peptide that naturally triggers proliferation and homeostasis of neurons. In the body, NGF binds with at least two types of receptors: the tropomyosine receptor kinase A (TrkA) and low-affinity NGF neurotrophin receptor p75 (LNGFR/p75NTR/p75). Both are associated with certain disorders in humans and animals, although the respective mechanisms of action are likely different. Several therapeutic applications for NGF have been proposed but few have matured to the market.

However, many therapeutic uses of NGF which have been envisaged in the past have not matured to marketed therapeutic NGF products, and one reason can be seen in that NGF, besides the desired effect on proliferation and homeostasis of neurons, is associated with pain: it can, when administered topically or systemically, cause hyperalgesia (Lewin et al., 1994, Eur. J. Neurosci., vol. 6, p. 1903-1912; Della Seta et al., 1994, Pharmacol. Biochem. Behav., vol. 49, p. 701; Dyck et al, 1997, Neurology, vol. 48, 501-505; McArthur, et al., 2000, Neurology, vol. 54, p. 1080-1088; Svensson et al., 2003, Pain, vol. 104, p. 241-247; Ruiz et al., 2004, Brain Res., vol. 1011, p. 1-6). As a solution, mutant versions of NGF (“muteins”) were developed, which are associated with reduced nociceptive activity (“painless NG”), and which are characterized by at least one mutation in the domain of NGF which interacts with the TrkA receptor (WO 2008/006893 A1, Malerba et al. PLOS One, 2015, vol. 10, e0136425). However, such polypeptides are so far not available to the public in pharmaceutically acceptable purity, and have not been proposed or developed for the treatment or prevention of dermatological disorders of the skin, possibly also in view of the prejudice and general negative experience with research on growth factors in this therapeutic field in general.

According to the present invention, the stability and thus the long-term purity of the polypeptide of SEQ ID NO: 3 or SEQ ID NO: 4 thereof can be obtained and/or improved by the aspects and embodiments described herein. Thus, the present disclosure not only makes a new treatment or prevention for a dermatological disorder available, but also provides the agent suitable for such treatment or prevention, at a purity grade suitable for therapeutic applications, including administration to a mammal. The agent of the present invention was not previously available to the public at such advantageous purity grade.

The polypeptide of SEQ ID NO: 3 or SEQ ID NO: 4 is not found in nature and can also be referred to as a non-natural polypeptide. Thus, the agent according to the present invention is not wild-type NGF, and in particular not wild-type human NGF.

Preferably, the non-natural polypeptide according to the present invention is provided at high purity. Optionally, the polypeptide comprises internal disulfide bridges. Optionally, the polypeptide is properly folded. Optionally, the polypeptide is soluble in an aqueous medium.

The present invention is, in part, based on experiments with two animal models of skin ulcers. In these models skin ulcers are induced in diabetic mice by circular biopsy punch or by cycles of pressure loading, and the polypeptide of the invention is topically applied. The polypeptide has induced a significant and dose-dependent improvement in the healing time of the ulcers in comparison with placebo treated animals. This improvement was evident at doses devoid of pain-related side-effects thus demonstrating a potential benefit over the state of the art.

In particular, data generated in vivo models of diabetic skin ulcers have demonstrated that the polypeptide of the present invention is painless, yet retains the activity of targeting the NGF receptor system, and thereby provides as a therapeutic means for the treatment or prevention of dermatological disorders. Indeed, the polypeptide of the invention retains the trophic properties of wild-type NGF on angiogenesis and re-innervation that favors ulcer healing without exerting the pro-nociceptive effects of wild-type NGF at the site of topical application and at the systemic level.

The present invention provides a polypeptide for use in the treatment and/or prevention of a dermatological disorder in a mammalian subject, wherein the polypeptide is selected among the polypeptide of SEQ ID NO: 3 and the polypeptide of SEQ ID NO: 4. Thus, the present invention also provides the polypeptide of SEQ ID NO: 3 or SEQ ID NO: 4 for use in a method for use in the treatment of the human or animal body by therapy, as described herein.

More particularly, the present invention relates to a specific therapeutic use of the polypeptide of SEQ ID NO: 3 and the polypeptide of SEQ ID NO: 4, wherein the specific therapeutic use is the treatment and/or prevention of a dermatological disorder in a mammalian subject. Thus, the present invention also provides the polypeptide of SEQ ID NO: 3 and the polypeptide of SEQ ID NO: 4 for use in a method for use in the treatment of the human or animal body by therapy, wherein the therapy comprises the treatment and/or prevention of a dermatological disorder in a mammalian subject. The mammalian subject is typically a subject characterized by the need of such treatment.

The polypeptide of SEQ ID NO: 3 as well as the polypeptide of SEQ ID NO: 4 is characterized by a mutation of the amino acid sequence of human NGF (hNGF, SEQ ID NO: 2), wherein said mutation is associated with reduced nociceptive activity. In particular, arginine at position 100 of hNGF is substituted by glutamic acid. The present invention is based, in part, on the surprising finding that a therapeutic effect can be achieved without the side effects known from the prior art.

Without wishing to be bound to a particular theory, it is preferred that the polypeptide according to the present invention comprises one or more disulfide bridges, and most preferably three disulfide bridges. Mature and properly folded mature human NGF is characterized by three disulfide bridges (linking positions 136↔201, 179↔229, 189↔231, position numbers refer to SEQ ID NO: 1; see Wiesmann et al., 1999, Nature, vol. 401, p. 184-188). Without wishing to be bound to a particular theory, it is preferred that the polypeptide according to the present invention comprises equivalent disulfide bridges (the position numbers of which are available to the skilled person by aligning the polypeptide according to the present invention with the polypeptide of SEQ ID NO: 1 and Wiesmann et al., supra.

### Description of Presence and Absence of Adverse Effects

Preferably, the treatment and/or prevention does not cause side effects or adverse effects in the subject to which the polypeptide is administered or has been administered. One side effect or adverse effect that is preferably absent in this context is hyperalgesia or pain. Thus, preferably, administration of the agent according to the present invention does not induce any hyperalgesic syndrome (pain).

It is important to point out that the absence of pain does not merely cause a more pleasant (or less unpleasant) treatment than the administration of a reference compound associated with pain (such as wild-type NGF), but is at least in part causative for the success of the treatment or prevention of skin disorders as such: considering that the polypeptide according to the present invention is preferably topically administered, more preferably topically administered onto the site of the skin disorder (e.g. ulcer), the absence of pain will enable the treated subject to accept the administration of the polypeptide onto the body surface without adverse reactions such as scraping it off or washing it off or otherwise removing it in order to use to pain, and as a result of that, the polypeptide will remain exposed to the wounded body surface an exert is therapeutically beneficial effect, such as treatment or prevention of the skin disorder. Thus, the absence of pain associated with the polypeptide of the present invention will be suitable to overcome consumer reluctance and concerns of the regulatory authorities. In other words, the absence of pain is associated with a significant increase in the benefit-to-risk ratio compared to agents that are associated with pain.

In particular, preferably, the treatment and/or prevention does not cause hyperalgesia in the mammalian subject. In one embodiment, the subject to which the polypeptide of the invention is administered does not suffer from mechanic allodynia. More precisely, mechanic allodynia is not induced in the subject to which the polypeptide of the invention is administered, so that the subject to which the polypeptide is administered does not suffer from mechanic allodynia.

In one embodiment, the subject to which the polypeptide of the invention is administered does not suffer from thermal allodynia. More precisely, thermal allodynia is not induced in the subject to which the polypeptide of the invention is administered, so that the subject to which the polypeptide is administered does not suffer from thermal allodynia.

A further side effect or adverse effect that is preferably absent in this context is malignancy or cancer. In particular, the administration of the polypeptide of the present invention to a subject is preferably not associated with abnormal cell growth, and even more preferably is not associated with abnormal cell growth with the potential to invade or spread to other parts of the body. It is particularly preferred that the administration of the polypeptide of the present invention to a subject is preferably not associated with cancer of the skin, in particular of the dermis or the epidermis. In this regard, the treatment or administration according to the present invention is associated with significant advantages compared to the state of the art, such as e.g. the commercial treatment with platelet derived growth factor (beclapermin, brand name Regranex). Thus, the absence of malignancy associated with the polypeptide of the present invention is expected to be suitable to overcome consumer reluctance and concerns of the regulatory authorities. In other words, the absence of malignancy is associated with a significant increase in the benefit-to-risk ratio compared to agents that are associated with malignancy.

Thus, in summary, preferably, the administration of the polypeptide of the present invention to a subject is not associated with adverse effects such as malignancy and/or pain.

Typically, administration of the agent according to the present invention is well tolerated by the subject. In particular, preferably, administration of the polypeptide according to the present invention is not associated with the formation of anti-drug antibodies in the subject. Indeed, as the amino acid sequence of the polypeptide according to the present invention differs in only one or two amino acid positions from wild type human NGF, it is plausible that the immunological tolerability in humans is particularly advantageous, and it is plausible that administration of the polypeptide of the present invention is not associated with the formation of anti-drug antibodies in humans.

Preferably, administration according to the present invention positively influences one or more of the following: inflammation, extracellular matrix deposition, innervation and angiogenesis.

### Detectability of the Polypeptide

Preferably, the polypeptide for use according to the present invention can be selectively recognized by a specific reagent with regard to endogenous (e.g. human) NGF. The terms “selectively recognized” and “detectable” are used interchangeably herein and generally refer to the specific identification, preferably by molecular means, of the protein, in a biological sample.

In that regard, the polypeptide according to the present invention is preferably detectable by an antibody or other immunoreactive molecule.

A protein detectable by an antibody or other immunoreactive molecule may also be referred to as an antigen. In some embodiments, a biological sample may be characterized by displaying—or not displaying—one or more specific antigens. In the context of the present invention, the polypeptide administered to the subject is preferably detectable in a biological sample obtained from the subject post administration of the polypeptide. One non-limiting way for showing presence of a protein is by Western Blot, but other immunological methods are equally comprised in the context of the present invention. The antibody or other immunoreactive molecule is either labelled (e.g. fluorophore-labelled) itself, or recognized by a labelled secondary antibody or other immunoreactive molecule, which is added for that purpose. Thus, in some cases, a secondary molecule that aids in the detection, such as e.g. an optionally labelled secondary antibody, is also added to facilitate detection.

According to the invention, an antigen is said to be present in a biological sample if the level is above the detection limit and/or if the level is high enough to allow binding by antigen-specific antibodies added to the sample. According to the invention, an antigen is said to be not expressed on a cell if the level of expression is below the detection limit and/or if the level of expression is too low to allow binding by antigen-specific antibodies added to the sample.

An antibody or other immune reactive molecule may recognize an epitope on the cell. The term “epitope” refers to an antigenic determinant in a molecule such as an antigen, i.e., to a part in or fragment of the molecule that is recognized, i.e. bound, by the immune system, for example, that is recognized by an antibody or other immunoreactive molecule. Detection of an epitope specific for any particular antigen normally allows to conclude that that particular antigen is present on the cell being analyzed.

In one embodiment, a sample obtained from a subject, in particular the subject to which the polypeptide according to the present invention has been administered, can be characterized by immunophenotyping. “Immunophenotyping” generally means that the cell or sample can be characterized by antigen-specific molecules such as antibodies or other immune reactive molecules, which are added to the sample to determine if an antigen is present. Immunophenotyping includes cell sorting using various methods including flow cytometry, as well as analytic methods on lysed cells and lysed samples, such as Western Blotting.

In the present invention, a polypeptide that can be specifically detected even in the presence of wild-type NGF, such as wild-type human NGF, is particularly preferred. While any mutation of an amino acid sequence, such as any point mutation, for instance, may render a polypeptide specifically detectable even in the presence of the respective non-mutated wild-type polypeptide, and therefore each of the polypeptide of SEQ ID NO: 3 and the polypeptide of SEQ ID NO: 4 may be prima facie specifically detected even in the presence of wild-type human NGF, it is particularly the polypeptide of SEQ ID NO: 4 for which an antibody is available that can distinguish said polypeptide from wild-type human NGF (WO 2008/006893 A1).

Thus, preferably the polypeptide is characterized by at least the absence of proline (which is present at position 61 of SEQ ID NO: 2, for reference) at position 61, more preferably by the substitution of proline at position 61 by another amino acid. In a particularly preferred embodiment, proline at position 61 is substituted by serine. In this preferred embodiment, the polypeptide for use according to the present invention is the polypeptide of SEQ ID NO: 4. This polypeptide is characterized by at least the absence of proline at position 61, more preferably by the substitution of proline at position 61 by another amino acid. In SEQ ID NO: 4, proline at position 61 of SEQ ID NO: 3 is substituted by serine.

### Wounded Body Surface

According to the present invention the wounded body surface is subjected to administration of the polypeptide of the invention.

Wounded body surfaces include, without limitation, ulcers (venous ulcers, arterial ulcers, pressure ulcers, diabetic ulcers), post-surgical wounds, bedsores, burns, lacerations, incisions, bruises, abrasions, puncture wounds) and the like. Subjects having such wounded body surface will be described further below, and the following description of wounded body surface is applicable to all such subjects, unless the context dictates otherwise.

In one embodiment, the agent according to the present invention is provided herein for the treatment of prevention of a skin disorder, wherein the skin disorder is selected from ulcers, post-surgical wounds, bedsores, burns, lacerations, incisions, bruises, abrasions and puncture wounds-

In one embodiment, the agent according to the present invention is provided herein for the treatment of prevention of an ulcer, wherein the ulcer is selected from a venous ulcers, arterial ulcers, pressure ulcers and diabetic ulcers.

In some embodiments the wounded body surface has a diameter of 1 mm or more. In general, when reference is made herein to the “diameter” of a wounded body surface, for non-circular wounded body surfaces, the term “diameter” refers to the largest diameter of the wounded body surface, measured from one border of the wounded body surface across the wounded body surface to the opposite border of the wounded body surface. For circular wounded body surfaces, the diameter is of course equal for any direction of measurement across from one border of the wounded body surface across the wounded body surface to the opposite border of the wounded body surface. The diameter can be determined with a ruler or other suitable means on the outer surface of the wounded body surface.

In some embodiments the wounded body surface has a diameter of 1 mm to 50 cm. In some embodiments the wounded body surface has a diameter of 2 mm to 20 cm. Wounded body surfaces with a diameter of 0.5 cm or more, preferably 1 cm or more, can also be referred to herein as “large” wounded body surfaces. The present invention is also suitable for the treatment of large wounded body surfaces, such as large ulcers (see e.g. Example 4). In some embodiments the wounded body surface has a diameter of 3 mm to 10 cm. In some embodiments the wounded body surface has a diameter of 4 mm to 5 cm. In some embodiments the wounded body surface has a diameter of 5 mm to 4 cm. In some embodiments the wounded body surface has a diameter of 6 mm to 3 cm. In some embodiments the wounded body surface has a diameter of 7 mm to 1 cm. In some embodiments the wounded body surface has a diameter of 8 mm to 1 cm. In some embodiments the wounded body surface has a diameter of about 6 mm. In some embodiments the wounded body surface has a diameter of about 12 mm.

Subjects for which the Agent According to the Present Invention is Particularly Suitable

According to the present invention, the polypeptide of SEQ ID NO: 3 or SEQ ID NO: 4 may be administered to a subject in need of such administration. A subject in need of such administration may be a subject suffering from a disorder described herein, a subject at risk of suffering from such a disorder, or otherwise afflicted with such a disorder. The agent is administered to the subject in a therapeutically effective amount. The therapeutically effective amount can be determined by the physician in view of the disclosure herein.

In particular, the polypeptide according to the invention is administered to a mammalian subject. The subject can also be referred to as “patient”. Most preferably, the mammalian subject is a human.

The present invention also relates to a method of treating a patient suffering from a dermatological disorder, wherein the method comprises administering an effective amount of the polypeptide of SEQ ID NO: 3 or the polypeptide of SEQ ID NO: 4 to the patient. The terms “patient” and “subject” are used interchangeably herein, particularly with reference to a patient/subject characterized by a dermatological disorder, as described herein.

Preferably, the dermatological disorder is characterized by wounded surface on at least a part of the body of the subject. Preferably, the dermatological disorder is characterized by wounded surface (wounded body surface). Wounded body surfaces have been described e.g. above, and the following description of subjects is applicable to all such wounded body surfaces on such subjects, unless the context dictates otherwise.

More preferably, the dermatological disorder is or comprises a skin lesion, preferably a skin lesion characterized by at least partial ablation of the dermis, and optionally of the dermis. In one embodiment the wounded body surface is or comprises a lesion, particularly a lesion of the skin.

Although terms like “dermatological disorder”, “wound”, “wounded body surface”, “chronic wound”, “ulcer” and other terms are used in the singular form herein, the present invention is also applicable to subjects having multiple dermatological disorders, wounds, wounded body surfaces, chronic wounds, ulcers and other such disorders.

Preferably, the wounded body surface is comprises at least one chronic wound. Thus, administration of the agent according to the present invention is suitable for the treatment or prevention of at least one chronic wound. In the context of the present invention, the term “chronic wound” is to be understood broadly and includes without limitations ulcers of all types, whether or not explicitly mentioned in this disclosure or not, bedsores, burns, mechanical skin ablations. In particular, wounds which fail to heal in the time windows typical for healing in healthy subjects of the respective species are comprised within the term. In addition to that, all wounds on a subject's body surface which have failed to heal and/or to close for seven days or more, such as 14 days or more, 21 days or more, 1 month or more, or one year or more, are included in the terms “chronic wound”. The agent according to the present invention may be administered to all such types of chronic wounds, in order to treat or prevent such chronic wounds.

In the context of the present invention, the term “prevent” is to be understood broadly and includes not only the prevention of onset of the disorder, but also the prevention of progression of the disorder. In particular, in the context of a wounded body surface, such as a chronic wound, e.g. an ulcer, the term “prevent also includes the prevention of further progression of the extension of the wounded body surface, such as further deepening of the wounded body surface and/or increase in diameter of the wounded body surface.

In the context of the present invention, the term “treat” is to be understood broadly and includes without limitation the amelioration of the symptoms of the disorder. Indeed, it is preferred and also demonstrated by the experimental examples herein that achieving amelioration of the dermatological disorder, such as e.g. (partial) closure of a wound, is a preferred integral part of the invention as claimed herein. Indeed, attaining the claimed therapeutic effect is a functional technical feature of the present invention. The examples herein make plausible that said functional technical feature is achievable as a direct result of administration of the polypeptide of the present invention. In other words, the present inventors have identified that the polypeptide of the present invention is causative for achieving amelioration in a subject suffering from a dermatological disorder. The dermatological disorder is preferably characterized by a wounded body surface.

The present invention is particularly suitable for a subgroup of subjects suffering from a dermatological disorder. Such subgroups are described herein. It is also possible that a particular subject falls into one or more of the subgroups described herein; administration of the polypeptide according to the present invention to subjects falling into one of the subgroups described herein is equally comprised by the present invention as administration of the polypeptide according to the present invention to subjects falling into more than one of the subgroups described herein.

The invention is not limited to particular causes of the wounded body surface. For example, diabetic causes are comprised in the invention as well as non-diabetic causes.

The wounded body surface may be on any one or more parts of the body. Preferred are wounded body surfaces on the extremities, such as the arms (including hands) and legs (including feet), but wounded body surfaces on the torso or head or other parts of the body may be subjected to administration of the polypeptide of the invention as well. In some embodiments, the wounded body surface is on a leg or foot, and more preferably on a foot. Such embodiments are frequent in diabetic subjects, but administration to such particular wounded body surface is not limited to diabetic subjects.

In some embodiments, the polypeptide according to the present invention is for administration to a subject who has undergone surgery. Accordingly, the polypeptide according to the present invention is suitable to treat or prevent the one or more postoperative complications such as bedsores and/or to treat surgical wounds.

Preferably, the wounded body surface comprises at least one ulcer. According to the present invention, the polypeptide may be administered to at least a part of the wounded body surface. “at least one part of”, as used herein, includes any ratio between 0 and 100%, such as between 10 and 90%, between 20 and 80%, between 30 and 70%, between 40 and 60%, and about 50%; thus, the polypeptide may be administered to the entire wounded body surface or to any part thereof. Optionally, the administration also includes the skin area adjacent to the wounded body surface.

Preferably, the dermatological disorder comprises at least one ulcer. According to the present invention, the polypeptide may be administered to at least a part of one ulcer. “at least one part of”, as used herein, includes any ratio between 0 and 100%, such as between 10 and 90%, between 20 and 80%, between 30 and 70%, between 40 and 60%, and about 50%; thus, the polypeptide may be administered to the entire surface of the ulcer or to any part thereof. Optionally, the administration also includes the skin area adjacent to the ulcer.

Diabetes mellitus is a common and debilitating disease that affects a variety of organs including the skin. It is currently estimated that between thirty and seventy percent of patients with diabetes mellitus, both type 1 and type 2, will present with a cutaneous complication of diabetes mellitus at some point during their lifetime. Irrespective to such theoretical considerations, which do not limit the present invention in any manner, methods for detecting diabetes are well known in the art. Methods of detecting diabetes are, in one embodiment, not part of the present invention, but they help in determining a subject subgroup that is of risk of suffering from a dermatological disorder, such as those described herein, and may this profit from treatment or prevention of such dermatological disorder in accordance with the present invention. In some embodiments, the agent according to the present invention is for administration to a diabetic subject suffering from neuropathy, such as in particular peripheral neuropathy. Methods for detecting neuropathy and predicting foot ulcer development in human beings with health conditions like diabetes mellitus are known (e.g. without limitation WO/2010/128519 A1).

The polypeptide according to the invention may be administered to a skin lesion in a diabetic subject, preferably a skin lesion characterized by at least partial ablation of the dermis, and optionally of the dermis, in such subject. In one embodiment the wounded body surface is or comprises a lesion, particularly a lesion of the skin of such subject. Preferably, the administration comprises administration to an ulcer, in particular a foot ulcer, in a diabetic subject.

Diabetic ulcers, in particular diabetic foot ulcers, are a major complication of diabetes mellitus. Within the context of the present invention, the term “diabetic ulcer” is not particularly limiting, apart from the precision that the ulcer is an ulcer in a diabetic subject. According to some estimates, diabetic subjects may have a five to fifteen times higher risk of non-traumatic amputation compared with non-diabetes (e.g. WO/2003/075949 A1). If untreated or not successfully treated, diabetic foot ulcers can be difficult to heal in some subjects and may even require amputation, particularly if accompanied by other complications or disorders, such as infection. Indeed, diabetes mellitus can affect multiple organ systems. Dermatologic manifestations of diabetes mellitus have various health implications ranging from those that are aesthetically concerning to those that, in untreated, may even be life-threatening. Dermatological implications of diabetes mellitus are described e.g. by Rosen et al., 2000, Endotext, De Groot et al., Eds., South Dartmouth (MA, USA), MDText.com, Inc. The present invention provides a treatment and/or prevention to such dermatological implications of diabetes.

In some embodiments, the polypeptide according to the present invention is for administration to a diabetic subject who has undergone surgery. Accordingly, the polypeptide according to the present invention is suitable to treat or prevent the one or more postoperative complications such as bedsores in a diabetic subject and/or to treat surgical wounds.

In general, besides diabetic ulcer, particularly diabetic foot ulcer, as well as bedsores and large/deep surgical wounds, may be difficult to heal even under medication, probably as a result of the large size of the areas involved. If these wounds are not treated in time, they will deteriorate and subsequently may become incurable and life threatening. The present invention provides a treatment and/or prevention to such dermatological implications of diabetes. Indeed, according to the present invention, an effective medical treatment may not-only help the patients recover from these skin complications, but may also lead them to a better quality of life, reduced medical care or expense, or even a prolonged life span.

The present invention is also suitable to treat wounded body surfaces, in particular ulcers, of large size, in diabetic and in non-diabetic subjects. In some embodiments, the present invention is suitable for the treatment of large wounded body surfaces with a diameter of 5 mm or more, such as 1 cm or more. Further details of the wounded body surface, including certain embodiments of the diameter of the wounded body surface, are described hereinabove.

Thus, the present invention provides an advantage over current treatment methods, which oftentimes may not be able to provide an effective method to treat large-area wounds. The present invention provides a treatment and/or prevention to such dermatological implications, including large-area wounds, in diabetic subjects and non-diabetic subjects.

According to the present invention, the polypeptide is suitable for the treatment or prevention of pressure injuries, including chronic pressure injuries. Pressure injuries, in particular, include pressure ulcers, pressure sores, decubitus ulcers and bedsores.

In preferred embodiments, the agent according to the present invention is for the use in treatment or prevention of an ulcer, and for that purpose, is administered to an ulcer. According to the present invention, the ulcer to which the polypeptide is administered is preferably selected from the group consisting of diabetic ulcers, trauma ulcers, surgical ulcers, pressure ulcers, chronic ulcers, and combinations of any of these ulcers. In specific embodiments the ulcers are selected from diabetic trauma ulcers, diabetic surgical ulcers, diabetic pressure ulcers, diabetic chronic ulcers, traumatic diabetic ulcers, traumatic surgical ulcers, traumatic pressure ulcers, traumatic chronic ulcers, chronic surgical ulcers, chronic pressure ulcers, and other ulcers. In some embodiments, the ulcers are selected from trauma ulcers, surgical ulcers, pressure ulcers and chronic ulcers in a diabetic subject. In some embodiments, the ulcers are selected from trauma ulcers, surgical ulcers, pressure ulcers and chronic ulcers in a non-diabetic subject.

The present invention is not limited to subjects having one ulcer nor to subjects having multiple ulcers. Among subjects having multiple ulcers, the present invention is not limited to treatment of only one of those ulcers nor to treatment of a certain number of those ulcers nor to treatment of all of those ulcers. Thus, the terms “ulcer” and “ulcers”, independent of their use in the singular or plural form in the present disclosure, are explicitly inclusive of all those embodiments and not limited to any specific number of ulcers on the subject nor any-specific number of ulcers being treated.

It has been established that foot ulceration in diabetes can either be associated with neuropathy (neuropathic ulcer), peripheral vascular disease (ischemic ulcer), or both (neuroischemic ulcer), although the final etiopathogenetic pathway may involve a combination of these primary risk factors and other causal factors such as trauma. Thus, in one embodiment, the polypeptide of the invention is for the prevention and/or treatment of ischemic ulcers, including ischemic foot ulcers. In an alternative embodiment, the polypeptide of the invention is for the prevention and/or treatment of neuropathic ulcers, including neuropathic foot ulcers. Finally, the polypeptide of the invention can be for the prevention and/or treatment of neuroischemic ulcers, including neuroischemic foot ulcers. All the aforementioned ulcers are optionally diabetic ulcers, although this is not a requirement.

In some embodiments, the polypeptide according to the present invention is administered to a subject suffering from ischemia. Ischemia may be local or systemic. In some embodiments, administration according to the present invention can reduce ischemia in the subject. Reduction of ischemia may be local or systemic.

In some embodiments, the polypeptide according to the present invention is administered to a subject suffering from neuropathy. In preferred embodiments, the agent according to the present invention is for administration to a subject suffering from neuropathy, such as in particular peripheral neuropathy. Such subjects may be diabetic or non-diabetic subjects. Indeed, it has been reported that a majority of diabetic ulcer patients have underlying neuropathy (Ndip et al., 2012, int. J. Gen. Med., vol. 5, p. 129-134). Thus, in a preferred embodiment, the polypeptide according to the present invention is administered to a diabetic subject suffering from neuropathy. Neuropathy may be local or systemic. In some embodiments, administration according to the present invention can reduce neuropathy in the subject. Reduction of neuropathy may be local and/or systemic. In one embodiment, reduction of neuropathy includes reduction of neuropathy in the area to which the polypeptide of the inventions is administered. In one embodiment, reduction of neuropathy includes reduction of neuropathy in the organ to which the polypeptide of the inventions is administered.

Treatment or prevention according to the present invention may be carried out by administration, preferably topical administration, of the polypeptide of the invention. In some embodiments, the administration is carried out at a hospital. In some embodiments, the treatment is not carried out a hospital.

Optionally but not mutually exclusive the dermatological disorder comprises at least one burn or a mechanical injury. Thus, the present invention also comprises the treatment or prevention of burn and mechanical injuries, whereby treatment of such injuries is practically more meaningful than prevention.

Preferably, the mammal to which the polypeptide of the invention is administered, preferably a human, suffers from diabetes mellitus or has a predisposition to suffer from diabetes mellitus; a respective subject is referred to herein as “diabetic subject”. In typical embodiments the diabetes mellitus is selected among diabetes mellitus Type 1 and diabetes mellitus Type 2.

Foot ulcers and other dermatological disorders are common in diabetic subjects. Such other dermatological disorders can be treated and/or prevented based on the present invention. One of the main causes of foot ulcers in diabetic subjects is neuropathy (nerve damage), making it difficult for the person to identify damage to their feet such as cuts, bruises, and pressure.

Thus, in one embodiment, the polypeptide is administered to a subject with foot ulcer. In one embodiment, the polypeptide is administered to a subject with diabetic foot ulcer (DFU). Indeed, foot ulcers and their attendant complications are frequent in subjects with diabetes, a majority of whom have underlying neuropathy (Ndip et al., 2012, Int. J. Gen. Med., vol. 5, p. 129-134). Such ulcers are also referred to as diabetic neuropathic foot ulcer. Thus, preferably the polypeptide is administered to a subject with diabetic neuropathic foot ulcer.

Optionally, administration of the polypeptide according to the present invention also comprises an aspect of improving the aesthetical appearance of the subject, in particular of the subject's body surface. In some embodiments wound closure is achieved as a result of the administration according to the present invention. In some embodiments scar formation is minimal. Thus, the administration according to the present invention also provides a cosmetic advantage to treated subject in comparison to untreated subjects. Therefore, the present invention also relates to a method of cosmetically treating a subject, wherein the method comprises the administration of the polypeptide according to SEQ ID NO: 3 or 4.

In another embodiment the agent according to the present invention is for the treatment or prevention of cancers on the skin, such as without limitation hemangiomas, and/or of skin disorders associated with such cancers.

In a further embodiment the agent according to the present invention is for the treatment or prevention of a dermatological disorder which results from a genetic disorder in the subject or is influenced by a genetic disorder in the subject.

### Administration

The present invention provides a heterologous polypeptide for administration to a subject.

Preferably, the polypeptide is for topical administration. Thus, preferably, the polypeptide of the invention is administered to the skin, or if the skin is wounded or absent, to the surface of the body at the site where skin would be found if it were not wounded or absent. In some embodiments, the polypeptide is administered onto the epidermis. In some embodiments, the polypeptide is administered onto the dermis. In some embodiments, the polypeptide is administered onto the tissue that normally is found under the epidermis, such as without limitation the subcutaneous area.

More preferably, the polypeptide is administered onto the wounded body surface. In other words, the polypeptide according to the present invention is preferably topically administered, more preferably topically administered onto the site of the skin disorder (e.g. ulcer).

The administration according to the present invention typically does not involve surgery of the subject. In one embodiment, administration of the polypeptide of the invention does not comprise or encompass an invasive step representing a substantial physical intervention on the body which requires professional medical expertise to be carried out and which entails a substantial health risk even when carried out with the required professional care and expertise. In contrast, in more typical embodiments, administration of the polypeptide of the invention, particular the topical administration, is generally considered safe for the subject and therefore the polypeptide may be administered by the subject himself or herself, particularly in case of a human subject.

Optionally, the ulcer is covered by a wound dressing prior to and/or during and/or after the administration. The enormous variety of types of wound dressings available is not limited by the present invention. Thus, any wound dressing may be used unless technically clearly inappropriate. In some embodiments, the polypeptide according to the invention is administered simultaneously to application of a wound dressing; optionally, the wound dressing comprises the polypeptide of the invention, optionally in the form of an aqueous medium applied to the wound dressing prior to administration.

Preferably the polypeptide is administered to a subject with foot ulcer onto the foot of said subject below the ankle.

In one embodiment, the polypeptide is administered in a single administration.

In an alternative and more preferred embodiment, the polypeptide is administered repeatedly. In a particularly preferred embodiment, the polypeptide is administered repeatedly one to five times per day. In one embodiment, the polypeptide is administered one time per day (see also Example 3). In one embodiment, the polypeptide is administered two times per day (see also Example 5). In one embodiment, the polypeptide is administered three times per day. In one embodiment, the polypeptide is administered four times per day. In one embodiment, the polypeptide is administered five times per day. It is particularly preferred that the polypeptide is administered to a human subject twice per day. All aforementioned administrations are preferably repeated over a course of several days, as disclosed herein. For example, the polypeptide may be administered repeatedly for a period of three to 30 days, preferably seven to 14 days, and preferably one to five times per each of these days.

In one embodiment, the polypeptide is administered repeatedly until closure of the wounded body surface. Alternatively, the polypeptide is administered in a single administration, and administration is discontinued after that single administration. Alternatively, the polypeptide is administered repeatedly for a period of three to 30 days, preferably seven to 14 days. Optionally, administration is discontinued after completion of said interval.

In some embodiments the agent is administered agent in such a manner that it is put into direct contact with nociceptive fibers (nerves). In some embodiments the agent is administered agent in such a manner that it is put into direct contact with fully exposed nociceptive fibers (nerves); nociceptive fibers (nerves) are considered fully exposed in cases of lack of skin, as is typical in cases of a wounded body surface. In some embodiments the agent is administered agent in such a manner that it is put into direct contact with hyperactivated nociceptive fibers (nerves); nociceptive fibers (nerves) are considered to be hyperactivated as a result of the skin lesion. In some embodiments the agent is administered agent in such a manner that it is put into direct contact with hyperactivated nociceptive fibers (nerves). Preferably, in such embodiments in particular, the agent does not cause a hyperalgesic syndrome (pain). No agent with such properties had been previously put at the disposal of the medical community. For this and other reasons, the present invention provides a major advantage.

### Dose

The agents and compositions described herein are administered in effective amounts. According to the present invention, an “effective amount” is the amount or dose which achieves a desired reaction or a desired effect, either alone or together with further doses. In the case of treatment of a particular disorder, the desired reaction preferably relates to inhibition of the course of the disease. This comprises slowing down the progress of the disease and, preferably, interrupting or reversing the progress of the disease. The desired reaction in a treatment of a disease or of a condition may also comprise a delay of the onset or a prevention of the onset of said disease or said condition. In some embodiments the desired reaction comprises the complete healing of the symptoms of the disorder, locally and/or systemically.

An effective amount of an agent or composition described herein will depend on the condition or disorder to be treated, the severity of the disorder, the individual parameters of the subject to which the agent is administered, such as age, physiological condition, accompanying condition(s) (if present), size and weight, the duration of treatment, the type of an accompanying therapy (if present), the specific route of administration and other parameters. Accordingly, the doses administered of the agents described herein may depend on various of such parameters. In the case that a reaction in a patient is insufficient with an initial dose, higher doses (or effectively higher doses achieved by a different, more localized route of administration) may be used.

According to the present invention, suitable and therapeutically effective dosages for the administration of a therapeutic agent for administration to a human subject for the treatment and/or prevention of a skin disorder, such as chronic cutaneous ulcer and burn wounds, can be determined based on experimentally determined suitable and therapeutically effective dosages for the administration of a therapeutic agent for administration to a rodent subject, particularly, a mouse, for the treatment and/or prevention of a skin disorder, such as chronic cutaneous ulcer and burn wounds. Guidance is available in “Guidance for Industry Chronic Cutaneous Ulcer and Burn Wounds—Developing Products for Treatment”, published by U.S. Department of Health and Human Services Food and Drug Administration, 2006.

Animal wound models (Examples 3 and 4) are helpful in establishing pharmacological responses, as well as assessing potential toxicities of wound-treatment products. In some embodiments the dose to be administered to the subject is a dose as disclosed in Example 3 or in Example 4 or in Example 5.

Preferably, the dose of the polypeptide to be administered is determined based on the surface of the wounded body surface to be treated. Preferably, the determination is conducted at the onset of treating. In one embodiment, the dosing is adjusted for later administration(s), depending on the surface of the wounded body surface at the time point of such later administration(s). In an alternative embodiment, the dosing is not adjusted for later administration(s), so that the dose of administration depends solely on the surface of the wounded body surface to be treated at the onset of the administration (first dosing), and subsequent dosages correspond to the first dose.

In one embodiment, the dose/each dose has an amount of 0.3 to 6 μg of the polypeptide per mm² of wounded body surface being treated (0.3 to 6 μg/mm²).

Optionally, in all embodiments according to the present invention, the dose is calculated based on the actual size of the wounded body surface (e.g. ulcer) at the time point of treatment. In other words, the dose may be subjected to calculation (recalculation) at every time point of administration based on the actual size of the wounded body surface (e.g. ulcer) at that time point.

### Process for Obtaining the Polypeptide

In one embodiment, the polypeptide of SEQ ID NO: 3 and the polypeptide of SEQ ID NO: 4 is obtainable from a biological source. Optionally, the polypeptide of SEQ ID NO: 3 and the polypeptide of SEQ ID NO: 4 is obtainable by recombinant expression. For that purpose, and open reading frame encoding the respective polypeptide is introduced into a source of recombinant proteins, for example a host cell or a cell-free system for protein expression. Indeed, considering that human NGF is produced only in minute quantities in vivo, mouse NGF is usually produced as a heterogenous mixture of various proteins (see WO 2000/022119 A1), and the polypeptides of the present invention are non-natural and thus not produced in vivo at all, the most meaningful possibility to produce the polypeptide of the present invention is by recombinant expression, in accordance to equivalent suggestions for wild-type NGF in the state of the art (WO 2000/022119 A1, WO 2008/006893 A1; Rattenholl et al., Eur. J. Biochem, 2001, vol. 268, p. 3296-3303, US 2018/0086805 A1). However, it has been a constant challenge to obtain such polypeptides at a purity grade sufficient for administration to a mammal. This challenge has been overcome by the present inventors, as disclosed in detail herein (see also Examples 1 and 2).

Preferably, the polypeptide according to the present invention is obtainable by recombinant expression in bacteria. More preferably, the polypeptide according to the present invention is obtainable by cytosolic recombinant expression in bacteria. In general, bacterial cells, in particular E. coli, are capable of recombinant production of high amounts of recombinant proteins, but, as is the case for many other recombinantly expressed genes, the production of recombinant NGF and similar polypeptides in bacteria results in a biologically inactive translation product which is then accumulated in the cell (cytosol) in the form of aggregates (so-called inclusion bodies (IBs) (WO 2000/022119 A1; US 2018/0086805 A1). In contrast to NGF, pro-NGF is known to be rather unstable and requires high efforts for refolding and purification at low recovery rates, which renders the process of NGF production via pro-NGF in bacteria relatively difficult and expensive. Thus, the main difficulties associated with bacteria-produced NGF and similar bacteria-produced polypeptides, via the respective pro-forms, concern the folding, the processing and the purification of the recombinant protein. These difficulties have now been solved (see Example 1 and 2). As a result, the polypeptides of SE ID NO: 3 and SEQ ID NO: 4 become available at a purity grade suitable for administration to a mammal, including a human.

Preferably, the polypeptide according to the present invention is expressed together with a pro-sequence. Without limitation, a suitable pro-sequence is the pro-sequence of wild-type human NGF (amino acid positions 18 to 121 of SEQ ID NO: 1), typically fused to the N-terminus of the polypeptide of SEQ ID NO: 3 or 4. For wild-type NGF, although not being part of mature NGF, and hence not required for the biological function of NGF, the presence of the covalently attached pro-sequence was shown to promote re-folding of recombinant NGF from inclusion bodies with concomitant disulfide bond formation of the mature part (beta-NGF). Thus, the presence of the covalently attached pro-sequence positively influences the yield and rate of re-folding when compared to the in vitro re-folding of mature NGF from inclusion bodies (Rattenholl et al., Eur. J. Biochem, 2001, vol. 268, p. 3296-3303). Without wishing to be bound to a particular theory, the same is plausible and postulated herein for the polypeptide of SEQ ID NO: 3 and 4.

Thus, when polypeptide according to the present invention has been produced in inclusion bodies, correct folding is required, and this is normally achieved post-translationally, as is the cleavage from the covalently attached pro-sequence; sophisticated methods for folding, cleavage and purification have been proposed in the past, in particular for wild-type human NGF. Notably, most of published studies on NGF apply a general refolding regime which was previously established by Rattenholl et al. (2001, Eur. J. Biochem, vol. 268, p. 3296-3303). Within this original study, several parameters of protein refolding (e.g. temperature, refolding time, pH of refolding reaction, arginine, glutathione and protein concentration) were investigated in detail and their effect on the refolding efficiency was assessed. The protocol by Rattenholl et al. relies on the re-naturation of the pro-form, which has a very poor solubility, obtainable from inclusion bodies after recombinant production in prokaryotes, whereby pro-NGF is solubilized in a solution of a denaturing agent in a denaturing concentration, transferred into a solution which is not or weakly denaturing, so that the solubility is maintained and the dissolved denatured pro-NGF can assume a biologically active conformation, including formation of disulfide bonds as in native NGF, and afterwards the NGF is purified and the pro-sequence is removed proteolytically (WO 2000/022119 A1; Rattenholl et al., Eur. J. Biochem, 2001, vol. 268, p. 3296-3303). Notably, within this study it was found that a low protein concentration leads to a higher specific yield of correctly folded product as compared to a higher protein concentration. Exemplary, protein concentrations around 50 mg per liter of refolding reaction resulted in a specific yield of ˜25% correctly folded pro NGF, while this fraction was reduced to 10% at protein concentrations of 500 mg per liter. Based on that, Rattenholl et al. suggest that the protein-concentration in the refolding solution has to be very low: according to Rattenholl et al., 15-20 mg of correctly folded protein per liter of refolding reaction are expected as yield. However, this would require a scale-up (e.g. beyond laboratory scale) for purification of even a few hundred mg recombinant protein.

While human pro-NGF contains a native cleavage site for the protease Furin (Arg¹-Ser²-Lys³-Arg⁴; R¹S²K³R⁴), and Furin cleaves pro-NGF at that site in vivo, Furin is not available at commercially relevant purity or quantity. According to the present invention, the polypeptide according to the present invention, when expressed together with a prosequence, e.g. in E. coli, is preferably cleaved by the protease Trypsin (EC 3.4.21.4), which is available commercially. Indeed, for wild-type NGF it has been reported that Trypsin would yield satisfying biologically active, mature NGF, which can be eventually purified (Rattenholl et al., Eur. 1. Biochem, 2001, vol. 268, p. 3296-3303), and Trypsin-based proteolysis of recombinantly expressed pro-NGF has meanwhile been adopted by others (e.g. D'Onofrio et al., 2011, PLoS One, vol. 6, e20839). However, it was later shown that cleavage of the wild-type pro-NGF with trypsin to produce beta-NGF is associated with several drawbacks, as low amounts of trypsin would lead to inefficient cleavage, whereas high amounts of trypsin would further decrease the selectivity of the cleavage, as trypsin is capable of cleaving C-terminally of any arginine and lysine residue (R and K residue), so that by digestion of R¹S²K³R⁴-containing pro-NGF by trypsin, several alternative digestion products would be obtained; thereby the use of trypsin as cleavage enzyme would lead to very low yields of correctly cleaved NGF, and to purification and yield problems, as the different cleavage products are not economically separated under standard conditions. As one solution, it was proposed to express a variant of pro-NGF, wherein the protease cleavage site R¹S²K³R⁴ in the pro-peptide is substituted at least at positions R¹ and K³ corresponding to positions 101 and 103 of the human wildtype pro-NGF sequence (SEQ ID NO: 1) by another amino acid (WO 2013/092776 A1). In one example, R¹ and K³, respectively, are replaced by valine (V) and alanine (A), transforming the original Furin cleavage site R¹S²K³R⁴ into V¹S²A³R⁴, wherein Trypsin is capable of cleaving specifically only C-terminally of R⁴; Trypsin-mediated cleavage of a respective pro-NGF can also be referred to as the “VSAR method”. Although WO 2013/092776 A1 is silent on then polypeptides of SEQ ID NO: 3 or 4 according to the present invention, the VSAR method has been initially proposed to be applicable to certain variants of pro-NGF muteins, although it was reported that the proteolysis conditions needed to be titrated with care (US 2018/0086805 A1). In the course of arriving at the present invention, the present inventors found that the VSAR technology, contrary to earlier suggestions, does not satisfactorily solve purity issues associated with the recombinant production of the polypeptide of the present invention at satisfactory purity. Indeed, the purification of recombinantly expressed beta-NGF or muteins thereof, not only from host cell proteins (HCP), but also from trypsin (or other protease used for cleavage) is still a challenge; needless to say, it would be required that a proteolytic enzyme (such as trypsin) be absent from a final preparation of a pharmaceutical protein, in order to avoid proteolysis during storage of the polypeptide, so that the polypeptide is substantially pure and un-degraded at the time point of administering it to a subject, according to the present invention. The present inventors have solved this challenge, as is described herein. Thus, the present invention makes the polypeptide according to SEQ ID NO: 3 or 4 available at high purity and thus essentially free of trypsin and/or of degradation products of the polypeptide. Although certain methods for the production of NGF (e.g. WO2013092776 A1) and of the polypeptide of SEQ ID NO: 4 (e.g. Malerba et al., 2015, PLOS One, vol. 10, e0136425) have been previously described, the present inventors discovered, surprisingly, that previously published processes are insufficient for obtaining the respective polypeptide at high purity. As a solution to these insufficiencies, the present inventors arrived at a new process and related aspects, as described in detail herein.

A process for obtaining the polypeptide of SEQ ID NO: 3 and the polypeptide of SEQ ID NO: 4 from recombinant expression, e.g. in a host cell, according to the present invention, may comprise purification. Purification, in the broadest sense, means that the polypeptide of SEQ ID NO: 3 or SEQ ID NO: 4 is separated from other molecules, including other proteins, such as host cell proteins. Thus, purification may include separation from one or more other molecules, including other proteins, such as host cell proteins, proteases (e.g. trypsin) and/or degradation products of the polypeptide according to the invention.

The process for production of the polypeptide of SEQ ID NO: 3 and the polypeptide of SEQ ID NO: 4 according to the present invention preferably comprises the following steps:
(a) obtaining a precursor of the polypeptide of SEQ ID NO: 3 or SEQ ID NO: 4,
(d) purification,
and the purification in step (d) typically comprises purification on a mixed mode stationary phase. Thus, in one embodiment, the polypeptide of SEQ ID NO: 3 or SEQ ID NO: 4 is obtainable by recombinant expression and purification, wherein the purification comprises purification on a mixed mode stationary phase. The term “on mixed mode stationary phase” is to be understood broadly and means that a mixture comprising the polypeptide of SEQ ID NO: 3 or SEQ ID NO: 4 or precursor of any of these, together with other molecular species, is exposed to a mixed mode stationary phase, e.g. by chromatography or other suitable process step. Indeed, preferably a mixture comprising the polypeptide of SEQ ID NO: 3 or SEQ ID NO: 4 or precursor of any of these, together with other molecular species, is subjected to chromatography, so that the purification in step (d) comprises purification by mixed mode chromatography. Preferably, the mixed mode chromatography comprises the use of a stationary phase having a charged group, preferably negatively charged group, and an aromatic group and/or a hydrophobic group.

Purification, in the broadest sense, according to the present invention, means that the polypeptide of SEQ ID NO: 3 or SEQ ID NO: 4 thereof is at least partially separated from other molecular species, including other proteins, such as host cell proteins, precursor and/or degradation products. As a result, the polypeptide of SEQ ID NO: 3 or SEQ ID NO: 4 which is at least partially purified is obtainable. While the other molecular species may be optionally discarded or not, the polypeptide of SEQ ID NO: 3 or SEQ ID NO: 4 is preferably obtained and retained as a result of the purification.

Preferably, the mixed mode chromatography comprises the use of a stationary phase having a charged group, preferably negatively charged group, and an aromatic group and/or a hydrophobic group.

Each of these steps may itself comprise several actions, which, for simplicity, can also be referred to as steps. For illustration, and as detailed below, step (d) may comprise more than one purification step, e.g. on more than one stationary phase.

Any letter or number used herein in relation to one or more process steps, such as e.g. (a), (b), (c), (d), (d1), (d2), is not to be understood as limiting, but rather for reference. It should not be understood that the sequence of events in the process or use according to the present invention may be limited by alphabetical sequence of letters or the numerical sequence of numbers; Notwithstanding the foregoing, it is strongly preferred that the sequence of events in the process or use according to the present invention is a sequence described herein.

Additional aspects of the mixed mode chromatography, particularly suitable stationary phases, will be described in some more detail below, but these aspects are generally applicable to the present invention. Thus, in particular all those stationary phases, including all embodiments thereof, that are described below to be particularly useful for the mixed mode chromatography in step (d2) are generally useful for the purification of the polypeptide of SEQ ID NO: 3 and/or the polypeptide of SEQ ID NO: 4 according to the present invention, and can be used in all types of embodiments, such as in combination with a step of (d1) capturing chromatography or without. Indeed, Example 2B describes that some advantages can be achieved by using mixed mode chromatography in a variation of a protocol according to the state of the art.

Optionally, the polypeptide of SEQ ID NO: 3 or the polypeptide of SEQ ID NO: 4 is obtainable in a process which comprises (re-)folding and/or chromatographic purification and/or protease digestion, and optionally adjustment to final protein concentration and/preparation of a desired formulation.

Thus, the administration of the polypeptide of SEQ ID NO: 3 or SEQ ID NO: 4 to a subject in need thereof as disclosed herein is also enabled through the industrially relevant purity and yield of the polypeptide of SEQ ID NO: 3 or SEQ ID NO: 4, which is available to the skilled person based on the disclosure herein. Thus, the present disclosure also describes a process for production of the polypeptide of SEQ ID NO: 3 or SEQ ID NO: 4.

The process for production of the polypeptide of SEQ ID NO: 3 or SEQ ID NO: 4 according to the present invention preferably comprises the following steps:
(a) obtaining a precursor of the polypeptide of SEQ ID NO: 3 or SEQ ID NO: 4, e.g. by recombinant expression,
(d) purification, wherein the purification comprises purification on a mixed mode stationary phase.

It is also preferred in the present invention that the precursor of the polypeptide of SEQ ID NO: 3 or SEQ ID NO: 4 is subjected to a step
(c) exposure to a protease.

Said exposure is typically carried out prior to step (d).

The process of the present invention is preferably also characterized in that no chromatographic purification is performed prior to the exposure to protease. Indeed, the present inventors have surprisingly found that the digestion with protease works well and efficient also in a crude fraction obtained from a host cell, i.e. when no chromatographic purification has been performed prior to the exposure to protease.

Preferably, the step of obtaining (a) comprises expression of a precursor of the polypeptide of SEQ ID NO: 3 or SEQ ID NO: 4, preferably recombinant expression. More preferably the recombinant expression is in a host cell. After culturing the host cell, the polypeptide of SEQ ID NO: 3 or SEQ ID NO: 4 is obtained in a fraction of the cell culture. The fraction may consist of the host cells, i.e. in case the protein is substantially not secreted from the host cells. This is the case e.g. when the polypeptide of SEQ ID NO: 3 or SEQ ID NO: 4 is produced in inclusion bodies and/or otherwise in an intracellular compartment including the cytosol. Suitable host cells can be selected from prokaryotic and eukaryotic host cells, although prokaryotic host cells are preferred in typical embodiments. Preferred prokaryotic host cells include Escherichia coli (E. coli), preferably E. coli Rosetta (DE3). In one embodiment, the polypeptide of SEQ ID NO: 3 or SEQ ID NO: 4 is obtained in a conformation other than the native conformation and/or in aggregates, most preferably in inclusion bodies. Then, preferably the process of the present invention comprises a step (b) of (re-)folding the polypeptide of SEQ ID NO: 3 or SEQ ID NO: 4. Preferably, step (c) is carried out after step (b).

Preferably, in step (c) the protease is a protease capable of cleaving the polypeptide of SEQ ID NO: 3 or SEQ ID NO: 4 in such a manner that the (mature) polypeptide of SEQ ID NO: 3 or SEQ ID NO: 4 is released. In a particular embodiment, said protease is trypsin, preferably porcine trypsin, optionally recombinantly expressed.

Preferably, the step of purification (d) comprises the following steps, preferably in sequential order:
(d1) capturing,
(d2) polishing.

Preferably, the step of capturing (d1) is carried out by chromatography, preferably column chromatography. More preferably, said step of capturing (d1) is carried out using a cation exchange chromatography stationary phase or a mixed mode chromatography stationary phase. Even more preferably said step of capturing (d1) is carried out using a mixed mode chromatography stationary phase, which is preferably Capto MMC.

Preferably, the step of polishing (d2) is carried out by chromatography, preferably column chromatography. More preferably, said step of polishing is carried out using a cation exchange chromatography stationary phase. Even more preferably said step of capturing (d1) is carried out using SP sepharose, preferably SP sepharose with a small particle size. SP is an abbreviation for sulfopropyl.

Optionally, the process according to the present invention comprises an additional step of adjustment to final protein concentration and/preparation of a desired formulation. As a result, a composition according to the invention is obtainable.

In other terms, the present invention provides mixed mode chromatography for the preparation of the polypeptide of SEQ ID NO: 3 or SEQ ID NO: 4. The mixed mode chromatography is useful in the preparation of the polypeptide of SEQ ID NO: 3 or SEQ ID NO: 4. In preferred embodiments, the precursor of the polypeptide of SEQ ID NO: 3 or SEQ ID NO: 4 thereof is exposed to a protease for the purpose of digestion, and the mixed mode chromatography is used in a step subsequent to exposure to the protease. In preferred embodiments, no chromatographic purification of the polypeptide of SEQ ID NO: 3 or SEQ ID NO:4 is performed prior to said exposure to protease.

### Purity of the Polypeptide

The polypeptide of the invention is substantially or essentially free from components that normally accompany it in its native state. The polypeptide of the invention is isolated before being administered. In one embodiment, the “isolated polypeptide” refers to the polypeptide, which has been purified from the cellular and extracellular environment, such as tissue, which surround it in a naturally-occurring state, e.g., from the cell in which it has been expressed, such as a host cell. In another embodiment, “isolated polypeptide” refers to in vitro isolation and/or purification of a polypeptide, respectively, from its natural cellular environment, and from association with other components of the environment in which the polypeptide normally resides.

Preferably, the polypeptide of SEQ ID NO: 3 or SEQ ID NO: 4 for use according to the present invention is substantially free of impurities. Such advantageously pure the polypeptide of SEQ ID NO: 3 or SEQ ID NO: 4 is obtainable as described herein.

The polypeptide of SEQ ID NO: 3 or SEQ ID NO: 4 described herein is regarded as a pharmaceutically active peptide or protein.

In a particularly advantageous embodiment of the present invention the polypeptide according to the present invention is obtained essentially free of degradation products of said polypeptide. In particular, the present inventors have observed that, contrary to reports on wild type human NGF in the state of the art, the exposure of a precursor of SEQ ID NO: 4 to trypsin will inherently partially cleave said precursor C terminally of arginine (Arg, R) residue 9 of SEQ ID NO: 4, either before or after purification, if purification does not completely remove trypsin (des-nona variant, data not shown). By the specific method of purification as provided in the present invention, the polypeptide according to the present invention can be obtained essentially free of trypsin and/or of the des-nona variant.

Preferably, the polypeptide obtainable as described above is essentially free of degradants of the polypeptide. In particular, the present disclosure makes the polypeptide of the invention available at a new, improved purity grade, and it is preferred that the polypeptide is administered at such high purity. Preferably, the polypeptide of the invention for use according to the invention is characterized by a purity grade of at least 90%. More preferably, the polypeptide of the invention for use according to the invention is characterized by a purity grade of at least 91%. More preferably, the polypeptide of the invention for use according to the invention is characterized by a purity grade of at least 92%. More preferably, the polypeptide of the invention for use according to the invention is characterized by a purity grade of at least 93%. More preferably, the polypeptide of the invention for use according to the invention is characterized by a purity grade of at least 94%. More preferably, the polypeptide of the invention for use according to the invention is characterized by a purity grade of at least 95%. More preferably, the polypeptide of the invention for use according to the invention is characterized by a purity grade of at least 96%. More preferably, the polypeptide of the invention for use according to the invention is characterized by a purity grade of at least 97%. More preferably, the polypeptide of the invention for use according to the invention is characterized by a purity grade of at least 98%. Even more preferably, the polypeptide of the invention for use according to the invention is characterized by a purity grade of at least 99%.

Most preferably, the polypeptide of the invention for use according to the invention is characterized by a purity grade of more than 99.0%, such as a purity grade of more than 99.1%, more than 99.2%, more than 99.3%, more than 99.4% %, more than 99.5%, more than 99.6%, more than 99.7%, more than 99.8% %, more than 99.9%.

Herein, “purity grade” generally refers to the weight (w) percentage of the polypeptide according to the present invention with respect to the weight (w) of biological material other than the polypeptide of the present invention. For illustration, in general, at a purity grade of 99.0%, the polypeptide of the present invention is present at a relative amount (weight) of 99.0 units (e.g. 1.0 mg), and the sum of the weight of all biological material other than the polypeptide of the present invention is 1.0 units (e.g. 1.0 mg). Such biological material other than the polypeptide of the present invention includes, without limitation, host cell proteins, nucleic acids, protease(s) such as e.g. trypsin, inactivated or not, degradation products of the polypeptide of the invention such as and other macromolecules of biological origin. In a particular embodiment, the “purity” grade refers to the purity vs. polypeptides other than polypeptides of the invention. For illustration, in that embodiment, at a purity grade of 99.0%, the polypeptide of the present invention is present at a relative amount (weight) of 99.0 units (e.g. 1.0 mg), and the sum of the weight of all polypeptides which are nonidentical to the polypeptide of the present invention is 1.0 units (e.g. 1.0 mg). Degradation products of the polypeptide of the present invention, for the avoidance of doubt, are included in the “polypeptides which are nonidentical to the polypeptide of the present invention”. A particular degradation product is the des-nona variant (see Examples 1 and 2).

In particular essentially free of the des-nona variant of the polypeptide. The des-nona variant is a previously uncharacterized-degradation product of the polypeptide of the present invention that is associated with production of certain variants of NGF including the polypeptide of the present invention, unless the polypeptide is produced by the new method disclosed herein (see e.g. Examples 1 and 2). “essentially free” in this context is intended to mean that the polypeptide of the invention for use according to the invention is characterized by a purity grade, with respect to the des-nona variant, of more than 99.0%, such as a purity grade of more than 99.1%, more than 99.2%, more than 99.3%, more than 99.4% %, more than 99.5%, more than 99.6%, more than 99.7%, more than 99.8% %, more than 99.9%, all with respect to the des-nona variant. In the most preferred embodiment, the des-nona variant is undetectable and/or absent.

It is also preferred that the polypeptide according to the present invention is essentially free of any protease (such as trypsin). “essentially free” in this context is intended to mean that the polypeptide of the invention for use according to the invention is characterized by a purity grade, with respect to the sum of all proteases (including trypsin), of more than 99.0%, such as a purity grade of more than 99.1%, more than 99.2%, more than 99.3%, more than 99.4% %, more than 99.5%, more than 99.6%, more than 99.7%, more than 99.8% %, more than 99.9%, all with respect to the sum of all proteases (including trypsin). In the most preferred embodiment, trypsin is undetectable and/or absent.

Such high purity grade, in the above-described embodiments, is associated with improved acceptability by regulatory authorities and qualifies the polypeptide of the present invention as a medicament for use in mammalian subjects, including humans, in particular. Thus, the purity grade according to the present invention enables for the first time the use of this polypeptide for administration to the wounded body surface, including the human wounded body surface, and ulcers, in particular, in a safe and reliable manner. The high purity grade with respect to protease (trypsin) in particular enables storage of the polypeptide also in non-frozen form.

### Compositions

In some embodiments, the polypeptide of SEQ ID NO: 3 or SEQ ID NO: 4 described herein is comprised in a composition comprising additionally one or more carriers and/or one or more excipients. The term “carrier” as used herein refers to an organic or inorganic component, of a natural or synthetic nature, which is combined together with the active ingredient in order to enable, enhance or facilitate application of the active ingredient. The term “excipient” as used herein is intended to indicate all substances which may be present in a pharmaceutical composition of the present invention and which are not active ingredients such.

Preferably the composition according to the present invention comprises at least water as an excipient. In some embodiments, the composition according to the present invention comprises aqueous media, and more preferably the composition according to the present invention is in the form of an aqueous solution. In one embodiment, the polypeptide is comprised in an aqueous medium, and the aqueous medium is administered to the mammalian subject. The aqueous medium may be for example an aqueous solution. Aqueous solutions and other respective compositions, in some embodiments, are obtainable directly from purification of NGF in aqueous media. For example, when the agent according to the present invention is obtained by purification form a biological source by purification, respective aqueous compositions may be obtainable directly from the last purification step, e.g. elution from the last chromatographic column (usually the polishing step) and/or filtration. Alternatively, respective compositions are available through an additional step of adjustment to final protein concentration and/preparation of a desired formulation. Such additional step may include, for example, a step of clarification or filtration, as described herein, and/or addition of one or more excipients and/or one or more carriers. Exemplary compositions useful in the present invention are described herein, without limitation.

Thus, the polypeptide of SEQ ID NO: 3 or SEQ ID NO: 4 described herein may be present in a composition, e.g. in a pharmaceutical composition. The compositions described herein are preferably sterile and preferably contain the polypeptide of SEQ ID NO: 3 or SEQ ID NO: 4 as a pharmaceutically active peptide or protein, and optionally of further agents, mentioned or not mentioned herein. The compositions may be in any state, e.g. liquid, frozen, lyophilized, etc.

The compositions described herein may comprise salts, buffer substances, preservatives, carriers, diluents and/or excipients, all of which are preferably pharmaceutically acceptable. The term “pharmaceutically acceptable” describes something non-toxic and/or which does not interact with the action of the active ingredient of the pharmaceutical composition.

Suitable buffer substances for use in the invention include acetic acid in a salt, citric acid in a salt, boric acid in a salt and phosphoric acid in a salt. For example, it is preferable that the polypeptide of the invention, as a result of the various aspects of the present invention, obtainable in a buffer having a pH between 4.5 and 6.5, preferably between 5.0 and 6.0. In one embodiment, an acetate buffer is a suitable buffer for such purposes, and is therefore particularly preferred. Thus, in one embodiment, the polypeptide of the invention is obtained in an acetate buffer having a pH between 4.5 and 6.5, preferably between 5.0 and 6.0.

Suitable preservatives for use in the compositions according to the present invention include those known in the art, among which are for illustration but without limitation benzyl alcohol, benzalkonium and its salts, M-cresol, phenol, chlorobutanol, paraben and thimerosal.

Thus, the present invention provides polypeptide of SEQ ID NO: 3 or SEQ ID NO: 4 for therapeutic use, i.e. for the use in a method of treatment of the human or animal body by therapy. Therapy may include prevention and/or treatment of a condition. In view of the potential for therapeutic use, said polypeptide can also be referred to as a pharmaceutically active protein or peptide.

Optionally the administration according to the present invention is accompanied by administration of at least one antimicrobial agent, such as an antibiotic. The antimicrobial agent may be part of the composition comprising the polypeptide according to the present invention, or alternatively may be administered to the subject separately, to the same or a different site, by the same or a different route of administration.

### INDUSTRIAL APPLICABILITY

The polypeptide of SEQ ID NO: 3 or SEQ ID NO: 4 described herein is suitable for a variety of purposes, e.g. for therapeutic applications as described herein.

The following examples and figures are intended to illustrate some preferred embodiments of the invention and should not be interpreted to limit the scope of the invention, which is defined by the claims.

### EXAMPLES

Materials and Methods Common to More than One Example

Unless specified otherwise, the following experimental examples concern specifically the polypeptide of SEQ ID NO: 4 characterized, with respect to wild-type human NGF, by the substitutions P61S R100E (termed “NGF P61S R100E”, Malerba et a. PLOS One, 2015, vol. 10, e0136425, SEQ ID NO: 4), as well as pro-forms etc. thereof. The polypeptide of SEQ ID NO: 4 may also be referred to as “NGF mutein”, but it has to be borne in mind that the therapeutic suitability, specific for this protein, according to this invention, and as demonstrated in the experimental examples, particularly Examples 3 and 4, is remarkably different from wild-type human NGF. Likewise, as described in Example 2, purification of the polypeptide of SEQ ID NO 4 differs from published purification protocols for wild-type NGF, and the specific process for preparing said polypeptide according to the present invention is suitable for achieving high purity, in particular absence of des-nona variant and trypsin.

The polypeptide of SEQ ID NO: 4 was recombinantly expressed as a precursor. To that end, SEQ ID NO: 4 was fused to the pro-peptide of wild-type human NGF (positions 1-121 of SEQ ID NO: 1). In other words, the precursor of the polypeptide of SEQ ID NO: 4 consisted of the precursor of human wild-type NGF (SEQ ID NO: 1), except for the substitutions P61S R100E in the mature portion of human wild-type NGF (but, for clarity, lacking the 2 most C-terminal amino acids of SEQ ID NO: 1 which do not form part of the polypeptide sequence of human wild-type NGF). Expression was performed in E. coli Rosetta (DE3) (strain: E. coli Rosetta (DE3)/pET11a-hpro NGF P61S R100E), in the form of insoluble inclusion bodies.

TABLE 1
List of Equipment used.
Equipment

| Device | Inventory-No. | Serial No. | Supplier |
|---|---|---|---|
| 1 L Bioreactors (incl. | E023, E024 | 07462/09, | Sartorius Stedim |
| sensors and pumps) | | 07463/09 | |
| 10 L Bioreactor | E082 | — | Sartorius Stedim |
| 300 V Power Source | E018; E019 | — | VWR |
| Äkta Explorer100a | E011 | 001054 | GE Healthcare |
| Äkta Explorer100a | E054 | 18111241 | GE Healthcare |
| Autoclave Systec | E050 | 2512 | Systec GmbH |
| VX-120 | | | |
| Centrifuge Galaxy 14D | E016 | 904090 | VWR |
| Centrifuge Sorvall | F683 | — | Sorvall |
| Evolution RC | | | |
| Clean bench | E006 | 40970929 | Thermo |
| | | | Scientific |
| Electrophoresis | — | — | Invitrogen |
| chamber Novex | | | |
| Mini Cell | | | |
| High pressure | F688 | 5-07.791 | APV |
| homogenizer | | | |
| APV 2000 | | | |
| HPLC, 1100 Series | E053 | — | Agilent |
| Magnetic stirrer | E013 | 30948231 | Heidolph |
| MR Hei-Mix S | | | |
| Magnetic stirrer | 686 | — | Corning |
| PC-620D | | | |
| pH-Meter inlab pH720 | E017 | 9080718 | WTW |
| Photometer | E051 | 2L9Q013008 | Thermo |
| Genesys 10uv | | | Spectronics |
| Pipetus | — | — | Hirschmann |
| | | | Laborgeräte |
| Pump VL 1000 | F606 | 0208004 | Verder |
| Scale | F651 | — | Sartorius |
| Scale | E030 | W092934 | Kern |
| Scale | E009 | — | Mettler |
| Shaker IKA KS 4000ic | E049 | — | IKA |
| Vortexer | E012 | 40934086 | VWR |

### Protein Parameters of Proteins and Peptides Described Herein

Theoretical values for protein parameters of relevant proteins were calculated with ExPASy's ProtParam-Tool, which is available at http://web.expasy.org/protparam. These are shown in Table 2, as follows:

TABLE 2
Theoretically deduced properties of relevant proteins/polypeptides

| | pro-form of SEQ ID NO: 4 | SEQ ID NO: 4 | porcine Trypsin |
|---|---|---|---|
| MW | 24.8 kDa | 13.23 kDa | 24.4 kDa |
| monomeric | | | |
| pl | 9.7 | 8.2 | 7.0 |
| ε | 25168 l/mol/cm | 19668 l/mol/cm | 34295 l/mol/cm |

### Analytical Methods

### SDS-PAGE and Western Blot

SDS-PAGE and Western Blots were performed using standard procedures. For SDS-PAGE, 12% Bis-TRIS NuPAGE gels (Article No. NP0342BOX from Thermo Fisher) were operated under reducing conditions at constant Volt (175 V) in NuPAGE MES-running buffer (Article No. NP0002 from Thermo Fisher). The primary antibody for Western Blot was purchased from Santa Cruz Biotechnology (NGF (H-20) sc-548). Examples of results are shown e.g. in FIG. 9A and FIG. 10.

### Analytical CEX-HPLC

CEX-HPLC was performed using a ProPac SCX-10 from Dionex. The column was operated with 50 mM citrate buffer, pH 5.5 at 1 mL/min. For elution, 1 M NaCl (B) was added and a linear gradient over 50 minutes from 0-100% B was executed. An example of results is shown in FIG. 98.

### SE-HPLC

SE-HPLC was performed using a Superdex 200 Increase 10/300 GL from GE Healthcare. The column was operated in PBS. Product was detected at 280 nm.

### Endotoxin, DNA and HCP

Endotoxin, DNA and host cell proteins (HCP) were determined according to standard protocols.

### Example 1: Expression of the Polypeptide of SEQ ID NO: 4 as a Precursor Protein

### Production Strain

The gene encoding for pro-NGF was cloned to pET11a expression plasmid. The gene was derived from H. sapiens and two point mutations (namely P61S and R100E) were introduced into the open reading frame. Subsequently, chemical competent Rosetta (DE3) cells were transformed with the expression plasmid and a single colony was selected (the resulting strain was termed E5901-STRAIN (=E. coli Rosetta (DE3)/pET11a-pro NGF P61S R100E

NGF RCB C-151101)). Aliquots were stored at <−60° C., in 1.0 mL.

In Example 1, initial fermentation development based on the strain E5901-STRAIN is described.

Equipment

| Device | Inventory-No. | Serial No. | Supplier |
|---|---|---|---|
| Autoclave Systec VX-120 | E050 | 2512 | Systec GmbH |
| Centrifuge Galaxy 14D | E016 | 904090 | VWR |
| Centrifuge Sorvall | F683 | — | Sorvall |
| Evolution RC | | | |
| Clean bench | E006 | 40970929 | Thermo |
| | | | Scientific |
| Magnetic stirrer | E013 | 30948231 | Heidolph |
| MR Hei-Mix S | | | |
| Magnetic stirrer PC-620D | 686 | — | Corning |
| pH-Meter inlab pH720 | E017 | 9080718 | WTW |
| Photometer Genesys 10uv | E051 | 2L90013008 | Thermo |
| | | | Spectronics |
| Pipetus | — | — | Hirschmann |
| | | | Laborgerate |
| Shaker IKA KS 4000ic | E049 | — | IKA |
| Weight Kern 572 | E030 | W092934 | Kern |
| Weight Mettler AE160 | E009 | — | Mettler |
| 1 L Bioreactors (incl. | E023, E024 | 07462/09, | Sartorius |
| sensors and pumps) | | 07463/09 | Stedim |

### Growth Media

### Complex Medium for Fermentation

The complex medium used for fermentation was composed of: 49.3 g/L yeast extract, 0.61 g/L MgSO₄*7H₂O, 0.5 g/L NH₄Cl, 14.2 g/L K₂HPO₄*3H₂O and 10 g/L glucose. The feed used for this fermentation was composed of 263 g/L yeast extract and 133 g/L glucose.

Minimal Media (MM) for fermentation

| | MM I-Final | MM |
|---|---|---|
| | conc. | II-Final |
| Constituent | [mM] | conc. [mM] |
| Aluminum chloride, hexahydrate | N/A | 0.000063 |
| Ammonium sulfate | 39.4 | N/A |
| Boric acid | 0.005 | 0.000125 |
| Calcium chloride, dihydrate | 2 | 0.000875 |
| Citric acid, monohydrate | 25.2 | 10 |
| Cobalt(II) chloride, hexahydrate | N/A | 0.00075 |
| Cobalt(II) sulfate, heptahydrate | 0.014 | N/A |
| Copper(II) sulfate, pentahydrate | 0.032 | 0.00425 |
| Diammonium phosphate | N/A | 35 |
| Dipotassium phosphate | N/A | 45 |
| Disodium hydrogen phosphate | 7.5 | N/A |
| Ferric chloride, hexahydrate | 0.37 | 0.17 |
| Kanamycin | 0.103 | 0.103 |
| Magnesium sulfate, heptahydrate | 4 | 3 |
| Manganese(II) sulfate, monohydrate | 0.142 | 0.00375 |
| Polypropylene glycol 2000 | N/A | N/A |
| Potassium chloride | 53.6 | N/A |
| Sodium chloride | 8.5 | 40 |
| Sodium dihydrogen | 31.9 | N/A |
| phosphate, monohydrate | | |
| Sodium molybdate, dihydrate | 0.001 | 0.00005 |
| Zinc sulfate, heptahydrate | 0.073 | 0.000375 |

For the batch phase, both basic media were supplemented with 30 g/L glucose. If not stated otherwise, the feed had the same composition as the respective batch medium, hut contained 300 g/L of the respective carbon-source.

LB-Agar Plates with Ampicillin and Chloramphenicol

LB-agar plates were freshly poured. The medium was composed of 10 g/L peptone, 5 g/L yeast extract, 5 g/L NaCl and 15 g/L agar. After autoclaving, the medium was supplemented with 100 μg/ml ampicillin and 30 μg/mL chloramphenicol.

### Fermentation

If not stated otherwise, fermentation, in this Example 1, fermentation was performed in 1 L stirred glass bioreactors controlled by a Biostat B unit from Sartorius. Typically, pO₂ was controlled to 30%, cultivation temperature was set to 37° C. and pH was controlled to 7 using 2 M phosphoric acid and 25% ammonium hydroxide. Unless stated otherwise, the batch phase was followed by an exponential feed with F₀=6 g/L/h and μ=0.25/h. For practical reasons, all exponential feeds were approximated by two linear feeds. Typically, induction of product expression was executed by addition of 1 mM IPTG and after induction, a constant feed rate of 10 g/L/h was applied. Cell biomass was harvested by centrifugation using a Sorvall Evolution RC from Thermo Scientific. The centrifuge was equipped with a SLC-6000 rotor and the culture was centrifuged at 8500 rpm and 4° C. for 30 min.

### Relative Quantification of Product in Biomass Samples

At given time-points, culture samples were diluted to an OD₆₀₀ of 10 and the biomass from 100 μL aliquots of this dilution was pelleted. Pellets were resuspended in 150 μl (non-reducing) Laemmli buffer and samples were boiled for 5 min. at 95° C. 10 μL of each sample were analyzed on a 10% Bis-Tris gel from Novex. Electrophoretic separation was performed for 90 min at 125 V and gels were stained with Coomassie. Destained gels were scanned and the abundance of the band corresponding to the precursor of the polypeptide of SEQ ID NO: 4 was quantified by densitometry. To further correct for variabilities in utilized biomass, the intensity of the band corresponding to the precursor of the polypeptide of SEQ ID NO: 4 was normalized to the intensity of a housekeeping protein.

Relative product accumulation was calculated from the increase of the band corresponding to the precursor of the polypeptide of SEQ ID NO: 4, post and pre-induction. Notably, the measured value represents a specific yield (i.e. normalized to an OD₆₀₀=10). For the absolute yield of a given fermentation, the actual cell-density has to be included in the consideration (cf. below).

### Absolute Quantification of Product in Biomass Samples

A standard for the precursor of the polypeptide of SEQ ID NO: 4 was obtained from the European Brain Research Institute (EBRI, Rome, Italy). The standard was diluted to a concentration of 65 μg/mL in Laemmli-buffer. The stated protein concentration was defined by EBRI. A standard curve was prepared with 260, 520, 780, 1040 and 1300 ng of the standard for the precursor of the polypeptide of SEQ ID NO: 4. Samples were analyzed on the same gel as the standard curve and dilution factor of the sample was considered to calculate the absolute product yield of product at the given time.

### Summary and Conclusions of Example 1

Based on the above, it is concluded that the production strain (ES901-STRAIN, cf. above) was successfully used for fermentation in 1 L scale. While different media compositions have been assessed for their ability to promote bacterial growth and product expression, minimal medium MM I supplemented with 5 g/L yeast extract proved to be favorable in terms of expression yield and obtainable cell density. In terms of product formation, no significant differences were observed, when the main culture was performed either with or without antibiotics (Ampicillin and Chloramphenicol, data not shown).

Example 1 can be up-scaled in order to produce the polypeptide at industrial scale.

### Example 2: Lab-Scale Purification, Establishment of Capto MMC

The precursor of SEQ ID NO: 4, used in this Example, was obtained in inclusion bodies as described in Example 1.

### Starting Point for Optimization in View of the State of the Art

At the onset, the present inventors reasoned that the process development could, in the absence of indications to the contrary, follow the basic cornerstones of NGF purification as previously reported in the literature. However, it was also borne in mind that, for an efficient production at large scale, adaptations suitable for a later scale up should be considered. Thus, it has been reasoned, based on Rattenholl et al. (supra), on WO2013092776 A1, and on other publications, that the polypeptide of SEQ ID NO: 4 may likewise be obtained at least at a lab-scale process, via its pro-form, employing unspecific digestion using trypsin and subsequent purification. It was reasoned that highly pure mature polypeptide of SEQ ID NO: 4 could be obtained thereby. However, it is only through the specific adaptions and modifications reported in this example that highly pure mature polypeptide of SEQ ID NO: 4 was obtained. Therefore, the administration of a polypeptide of SEQ ID NO: 4 to a subject in need thereof is enabled particularly in view of the high purity of the polypeptide of SEQ ID NO: 4 as described herein.

Equipment, production of the polypeptide of SEQ ID NO: 4

| Device | Inventory-No. | Serial No. | Supplier |
|---|---|---|---|
| 1 L Bioreactors (incl. | E023, E024 | 07462/09, | Sartorius |
| sensors and pumps) | | 07463/09 | Stedim |
| 300 V Power Source | E018; E019 | — | VWR |
| Äkta Explorer100a | E011 | 001054 | GE Healthcare |
| Äkta Explorer100a | E054 | 18111241 | GE Healthcare |
| Autoclave Systec VX-120 | E050 | 2512 | Systec GmbH |
| Centrifuge Galaxy 14D | E016 | 904090 | VWR |
| Centrifuge Sorvall | F683 | — | Sorvall |
| Evolution RC | | | |
| Clean bench | E006 | 40970929 | Thermo |
| | | | Scientific |
| Electrophoresis chamber | — | — | Invitrogen |
| Novex Mini Cell | | | |
| High pressure | F688 | 5-07.791 | APV |
| homogenizer APV 2000 | | | |
| HPLC, 1100 Series | E053 | — | Agilent |
| Magnetic stirrer | E013 | 30948231 | Heidolph |
| MR Hei-Mix S | | | |
| Magnetic stirrer PC-620D | 686 | — | Corning |
| pH-Meter inlab pH720 | E017 | 9080718 | WTW |
| Photometer Genesys 10uv | E051 | 2L9Q013008 | Thermo |
| | | | Spectronics |
| Pipetus | — | — | Hirschmann |
| | | | Laborgeräte |
| Pump VL 1000 | F606 | 0208004 | Verder |
| Scale | F651 | — | Sartorius |
| Scałe | E030 | W092934 | Kern |
| Scale | E009 | | Mettler |
| Shaker IKA KS 4000ic | E049 | — | IKA |
| Vortexer | E012 | 40934086 | VWR |

List of Equipment used in Example 2.

Details of the manufacturing process according to this Example, including improvements described in Example 2B3, are given in the process overview in FIG. 1.

Unless specified otherwise, analytical methods were as described in the above section “Analytical methods”.

### Example 2a. Purification Based an Previously Described Protocols

E. coli cells expressing the precursor of the polypeptide of SEQ ID NO: 4 (“biomass”) were produced as described in Example 1, and cells were lysed by addition of lysozyme and subsequent sonication on ice. Inclusion bodies (“IBs”) were (1) extracted from the host cells and washed with 6% Triton X100 (in 1.5 M NaCl, 60 mM EDTA) and, and (2) solubilized in 6 M guanidinium HCl (“gHCl”), 0.1 M Tris-HCl pH 8.0, 1 mM EDTA, 100 mM (fresh) DTT. IBs were solubilized for 2 h at room temperature. Afterwards, the pH was lowered to 3-4 by addition of 37% HCl. The thus obtained solution comprising solubilized precursor of the precursor of the polypeptide of SEQ ID NO: 4 (“solubilizate”) was dialyzed against 6 M gHCl (pH 3-4).

Refolding of the precursor of the polypeptide of SEQ ID NO: 4 was performed in 0.1 M Tris-HCl, 1 M L-arginine, 5 mM EDTA, 0.61 g/L oxidized glutathione and 1.53 g/L reduced glutathione, pH 9.5 at +4° C. Therefore, 50 μg of protein were added per mL of refold buffer, each hour. After refolding, the reaction was dialyzed against 50 mM sodium phosphate pH 7.0. While the buffer was exchanged, significant precipitation occurred.

The precursor of the polypeptide of SEQ ID NO: 4 was purified over a consecutive sequence of cation-exchange chromatography (SP Sepharose HP operated with 50 mM sodium phosphate, pH 7.0 and eluted with a NaCl-gradient) and subsequent hydrophobic-interaction chromatography (Phenyl Sepharose HP, operated with 50 mM sodium phosphate, 1 M ammonium sulfate pH 7.0). Afterwards, another dialysis was employed to exchange the sample's buffer against 50 mM sodium phosphate, pH 7.0 (note that such second dialysis could, however, be omitted in the process of Example 5). Again significant amounts of product did precipitate throughout the process of reduction of the buffer's conductivity.

The thus prepared precursor of the polypeptide of SEQ ID NO: 4 was subjected to limited proteolysis by adding 1 mg trypsin per 250 mg pro-NGF. The exposure of the precursor of the polypeptide of SEQ ID NO: 4 to the protease was for 14 h at 2-8° C.

The matured NGF was finally polished over a cation-exchanger (SP Sepharose XL operated with 50 mM sodium phosphate, pH 7.0 and eluted with a NaCl-gradient). Finally, product was concentrated to 0.5-1 mg/mL and was frozen at <−65° C.

### Example 2B: Improvements

In the following, several improvements, compared to Example 2A, as tested and implemented by the present inventors in the course of arriving at the present invention; are described. Unless the context dictates otherwise, all those details which are not expressly indicated were as described above for Example 2A.

### Optimization of IB-Solubilization

While low amounts of IBs (as exemplary received from shaking flask cultures) had been previously reported to be readily solved in the solubilization buffer (6 M gHCl, 0.1 M Tris-HCL pH 8.0, 1 mM EDTA, 100 mM (fresh) DTT), the IBs obtained from high cell-density fermentations could not be resolved entirely. This could be solved, by the present inventors, by addition of 2 M urea to said solubilization buffer, which turned out to improve the solubilization yield significantly (data not shown). For the avoidance of doubt: the 2 M urea were present in addition to the 6 M gHCl and other ingredients.

### Refolding Optimization

It was decided, initially based on Rattenholl et al. (2001, Eur. J. Biochem, vol. 268, p. 3296-3303; Rattenholl, 2001, Dissertation zur Erlangung des akademischen Grades doctor rerum naturalium (Dr. rer. nat.), Martin-Luther-Universität Halle-Wittenberg (Germany)), but importantly also taking into consideration a later (up)scalability, to perform the refolding with 200 to 500 mg of the precursor of the polypeptide of SEQ ID NO: 4 per liter of refolding reaction, preferably 200 to 300 mg of the precursor of the polypeptide of SEQ ID NO: 4 per liter of refolding reaction. This lead to relatively good yield of solubilized precursor of the polypeptide of SEQ ID NO: 4. In particular it important to consider that by such increased amount of NGF compared to the volume of refolding reaction, and under consideration that the refolding reaction comprises relatively expensive ingredients such as glutathione and arginine, relatively more the precursor of the polypeptide of SEQ ID NO: 4 could be refolded per volume of refolding reaction, which should render the refolding economically feasible, also at production scale.

### Purification of the Precursor of the Polypeptide of SEQ ID NO: 4

Purification of the precursor of the polypeptide of SEQ ID NO: 4, after refolding, was done by an approach which utilizes the rather high isoelectric point of pro-NGF and employs a cation exchange stationary phase (namely SP Sepharose) for purification. In order to run this type of chromatography, for technical reasons, the refolding buffer has to be exchanged against a buffer with low conductivity. While doing this, significant quantities the precursor of the polypeptide of SEQ ID NO: 4 precipitated (data not shown). This observation could be attributed to the reduction of the arginine concentration in the buffer.

Therefore, some efforts were taken to replace the capture column by a different one (a column with different selectivity), which could be more tolerant to the presence of arginine in the refolding reaction. In a first attempt to do this, the performance of several r stationary phases was assessed, but none of the approaches resulted in promising results (cf. Table 6). Therefore, the stationary phase used for the capture column was kept as it was defined by the previous process. However, due to the high isoelectric point (pi) of the precursor of the polypeptide of SEQ ID NO: 4, an increase in the conductivity of the running buffer (by addition of 250 mM L-arginine) was possible without affecting the performance. By this, refolded precursor of the polypeptide of SEQ ID NO: 4 could be stabilized to a certain extent and the amount of precipitated precursor was reduced (data not shown).

| Potential | | |
|---|---|---|
| capture step | Short description | Evaluation |
| Hydrophobic | Hydrophobic interaction | If ammonium sulfate |
| interaction | chromatography is not | or sodium chloride |
| chroma- | susceptible to conductivity | were added to product |
| tography | and salt composition | present in the refolding |
| | of the loaded sample. | buffer, protein precipitated |
| | | heavily. Even addition of |
| | | small quantities of ammonium |
| | | sulfate (to a concentration |
| | | of 0.25M) led to |
| | | precipitation. Also addition |
| | | of sodium chloride was not |
| | | possible without provoking |
| | | precipitation of product. |
| | | Therefore, a capture |
| | | step based on Phenyl- |
| | | or Butyl-Sepharose |
| | | is not an option. |
| Mixed-mode | Capto MMC is a | Within an initial set-up, a |
| chroma- | mixed-mode stationary | phosphate buffer at pH 5.5 |
| tography | phase combining | was supplemented with |
| | hydrophobic properties | 0.25M L-Arginine and elution |
| | with those of a | was facilitated with a NaCl- |
| | stationary phase | gradient. However, no |
| | for cation-exchange. | significant amounts of |
| | Because binding | product (precursor) |
| | is not solely | were recovered by |
| | mediated by ionic | this approach. |
| | interactions, this stationary | |
| | phase is more salt-tolerant | |
| | than classical cation- | |
| | exchange stationary phase. | |
| Size | Size exclusion | Although the preparative |
| exclusion | chromatography has a | chromatogram looked |
| chroma- | good resolution and is | promising, no separation of |
| tography | independent from the | the precursor of the |
| | sample-buffer. | polypeptide of SEQ ID NO: 4 |
| | The typical bottleneck | from impurities could be |
| | of this type of | achieved. This may |
| | chromatography (i.e. its | be caused by the existence |
| | limited capacity) is not | of this specific precursor |
| | applicable since relatively | as a polydisperse |
| | low amounts of product | mixture under the investigated |
| | are requested. | conditions. |

Table above: Alternative selectivities tested for capture of the precursor of the polypeptide of SEQ ID NO: 4, and evaluation thereof.

Regarding mixed mode chromatography, it is understood by the inventors, however without wishing to be bound to a particular theory, that the precursor of the polypeptide of SEQ ID NO: 4 does not elute efficiently from mixed mode chromatography, whereas the mature polypeptide of SEQ ID NO: 4 does.

### Protease Digestion to Yield Mature NGF

For manufacture of the polypeptide of SEQ ID NO: 4, the protease (trypsin) is essential and therefore, it was reasoned that ideally, the particular trypsin selected should meet the following criteria:
1. Derived from a recombinant source. Certification of animal-free raw material is pivotal for later on required GMP-compliance of the process.
2. Low side-activity of trypsin. Notably, trypsin can be subjected to autolysis. This process may result in so called pseudotrypsin, which has a broadened substrate-spectrum and possesses chymotrypsin-like activity. Ca²⁺ (e.g. 1 mM CaCl₂)) may be added to reduce autolysis. However, nowadays typically “modified trypsin” is applied for every protocol, which requires a tight sequence specificity (e.g. for peptide finger printing). This modified trypsin is typically obtained by acylation of trypsin's exposed ε-amino groups of lysine residues.
3. Low batch-to-batch variability, in order to enable a reproducible production process. Alternatively, the chosen enzyme should be delivered with a certificate stating the specific activity of the respective batch. The required amount of enzyme may then be based on activity rather than on mass.

Despite a comprehensive search, no trypsin fulfilling both criteria 1 and 2 was identified on the commercial market. It was reasoned that criterion 1 is more important. To reduce autolysis, addition of CaCl₂ may be sufficient. As a result, a recombinant ‘GMP grade’ trypsin from Roche (Roche 06369880103, Lot: 11534700) was chosen as raw material for the process. The sequence of this enzyme, which is expressed in Pichia pastoris, was derived from Sus scrofa. According to its certificate, the utilized trypsin batch has a specific activity of 4997 U/mg (determined according to USP).

### Omission of a Second Purification Step Prior to Trypsinization

Within an initial screen searching for optimal enzyme/substrate ratios for the intended trypsinization, the precursor of the polypeptide of SEQ ID NO: 4 obtained from the capture column (see above) was used. In contrast to a previously established process (European Brain Research Institute (EBRI), details not published, based on Rattenholl et al., supra) the present inventors decided not to use an additional hydrophobic interaction chromatography prior to trypsinization. The decision to omit such a second column purification step prior to trypsinization was mainly based on two lines of thinking: On the one hand; product obtained after the capture column was already virtually pure according to SDS-PAGE. On the other hand, the trypsinization itself may help to improve the impurity-profile by digestion of remaining host cell proteins (HCPs).

Table 7 summarizes the matrix of conditions screened within the first round. Results of trypsinization were investigated 12% SDS-PAGE (data not shown). The results (data not shown) indicate that trypsinization reproducibly yields stable polypeptide of SEQ ID NO: 4 over a rather wide range of enzyme/substrate-ratios (i.e. from 1-5 μg trypsin per 375 μg precursor of the polypeptide of SEQ ID NO: 4). Timing of digestion is not highly critical. Therefore, stopping of the reaction and the time required to load the reaction to the polishing column is apparently not limiting. This finding is of special importance since the reaction cannot be suitably or economically quenched at preparative scale.

Some additional experiments were conducted in order to refine an optimal enzyme/substrate ratio for the envisaged trypsinization, and it was found that with an enzyme/substrate ratio of 1/100 to 1/200 (protein weight/protein weight), good yields of the polypeptide of SEQ ID NO: 4 on the one hand and low amounts of truncated products on the other hand could be obtained reproducibly. It has to be stated that under the utilized conditions (i.e. within phosphate/arginine buffer (pH 7.0) at 2-8° C. and incubated (exposed to protease) for two to six hours) the quality of the digestion was not highly dependent on the enzyme/substrate ratio. This finding is of special importance since the underlying enzymatic digestion is prone to minor variations in the experimental set-up (e.g. alteration of trypsin's activity due to batch-to-batch variability or storage of the enzyme; timing and temperature of the incubation step (exposure to protease); errors in determination of protein concentrations). Moreover, this is also the reason why extended fine-tuning at small scale to further reduce potential truncation products seems to be not meaningful. If an “optimal” condition would be identified in small scale, there is still a good chance to produce a slightly changed product-pattern the next time virtually the same digest is repeated at larger scale.

Polishing Chromatography with the Aim to Obtain Pure Polypeptide, Following Trypsinization

In contrast to a previously established process (European Brain Research Institute (EBRI), details not published, based on Rattenholl et al., supra), which employed a SP Sepharose stationary phase for polishing of mature polypeptide (note: SP sepharose is a cation exchange stationary phase), here a more suitable stationary phase was searched for, based on the following considerations: in order to be efficiently loaded to an SP Sepharose column, a reduction of the conductivity of the solution comprising the precursor of the polypeptide of SEQ ID NO: 4, such as by buffer exchange is required. It is however known (e.g. Example 2A) that reduction of the ionic strength of the solution does result in precipitation of the target molecule and therefore, a buffer exchange to a low conductivity buffer should be avoided. Moreover, a cation exchange stationary phase was already used for capture of the precursor of the polypeptide of SEQ ID NO: 4, and an orthogonal selectivity is preferred in order to achieve a better separation of remaining contaminants. A third and final argument against the use of a SP stationary phase for purification of the trypsinization reaction is that potentially remaining precursor of the polypeptide of the precursor of the polypeptide of SEQ ID NO: 4 would bind to this column and could be separated from mature polypeptide of SEQ ID NO: 4 merely by elution selectivity and not by binding selectivity.

In order to establish such an orthogonal polishing column for purification of mature polypeptide of SEQ ID NO: 4, it was intended to use a hydrophobic interaction (HIC) column in a first instance. This stationary phase was not only chosen to have an orthogonal selectivity, but also because a buffer exchange to a low conductivity buffer is not necessary. Despite testing of several HIC stationary phase and conditions (e.g. Phenyl- and Butyl-Sepharose operated with 1 M (NH₄)₂SO₄ and 0.5 M (NH₄)₂SO₄, respectively), no satisfying polishing step based on HIC could be implemented (data not shown).

However, in a further experimental setup for a polishing step, the mixed mode stationary phase Capto MMC was tested and could be implemented successfully. It was found that with optimized conditions, the stationary phase binds to the polypeptide of SEQ ID NO: 4 reversibly and the product can be eluted by increasing pH (data not shown). In contrast, the precursor of the polypeptide of SEQ ID NO: 4 binds irreversibly onto the stationary phase and can be only eluted by using 1M NaOH as mobile phase (data not shown). Furthermore, it could be shown that trypsin does not bind at all onto the column operated at the same conditions (data not shown). These results provide clear evidence that the Capto MMC stationary phase is capable of efficiently separating mature polypeptide of SEQ ID NO: 4 from trypsin and from remaining precursor of the polypeptide of SEQ ID NO: 4.

### Establishment of an Additional Membrane Chromatography

In order to further deplete endotoxins and DNA, an additional anion-exchange membrane was included in the process. In general, and as is commonly known, membrane chromatography is characterized in that a solution comprising a component to be analyzed or purified (in the present case the polypeptide of SEQ ID NO: 4) is passed over or through a membrane, which is normally charged. For that purpose, in the present case a STIC-membrane (Sartorius, Goettingen, Germany) was incorporated at the positions indicated in FIG. 1. It could be shown that the polypeptide of SEQ ID NO: 4 does not bind to the membrane, and thus, a proof of concept was provided that membrane chromatography is suitable for purification of the polypeptide of SEQ ID NO: 4. For illustration of the incorporation in the entire process, including membrane chromatography, see FIG. 1.

### Reproducibility of the Process According to Example 2

In order to probe the robustness of the process, the process was conducted five times and resulting fractions were analyzed with respect to their yield and purity. Throughout these runs, a steady optimization of process details was pursued and buffer composition, gradients and so on were adopted until the final, optimized process details (see FIG. 1) were established. The results indicate that in lab-scale approx. 50 to 100 mg polypeptide of SEQ ID NO: 4 can be yielded from one consistent production run. Notably, the product obtained was consistently found, by SDS polyacrylamide gel electrophoresis followed by Coomassie staining or silver staining, to be relatively pure (less than five percent of contaminating host cell proteins and only traces of truncated NGF, data not shown).

For the precursor of the polypeptide of SEQ ID NO: 4 no meaningful method for SE-HPLC could be established. In contrast, SE-HPLC analysis for mature polypeptide of SEQ ID NO: 4 was straightforward and resulted in a homogeneous product peak of approx. 16 kDa which fits with a monomeric state of the polypeptide of SEQ ID NO: 4 n (data not shown).

### Summary and Conclusions

For this process, refolded precursor of the polypeptide of SEQ ID NO: 4 was captured using a SP Sepharose FF (“FF” stands for Fast Flow, i.e. a stationary phase with relatively large particles) and was subsequently treated with trypsin to yield mature NGF. For that purpose, the arginine concentration of the refolding reaction was decreased from 1 M (as recommended by the prior art) to 350 mM.

Control of the proteolytic cleavage of the precursor of the polypeptide of SEQ ID NO: 4 to yield mature polypeptide of SEQ ID NO: 4 is considered as most critical factor for the process. Herein, conditions were identified to reproducibly facilitate cleavage with high efficiency on the one hand and prevent formation of degradation products of NGF. The experimental data herein have shown that an apparently robust production process can be established over a rather wide range of enzyme/substrate ratios. For the trypsinization, step yields are apparently good and no significant loss is expected at this stage of the process. The product pattern obtained does apparently not strongly depend from the used reaction conditions (in terms of enzyme/substrate-ratio and time of incubation (time of exposure to protease)). Notably, even if a good yield for polishing of the enzyme is expected, at least 2*x grams of the polypeptide of SEQ ID NO: 4 have to be processed in order to deliver x gram of mature polypeptide of SEQ ID NO: 4.

The purification according to this example is a lean process consisting of merely two chromatographic purification steps. The existing purification process was further optimized and several aspects were adopted for scale-up (see FIG. 1). Exemplary, previously used methods of cell disruption were replaced by high-pressure homogenization and all dialysis steps could be replaced by tangential-flow filtration. The thus established process is capable to deliver the polypeptide of SEQ ID NO: 4 at high purity.

Despite the named challenges, the overall process seems to be capable of delivering product which appears to be of acceptable quality.

The complete process incorporating the improvements according to Example 2, including membrane chromatography, is schematically depicted in FIG. 1.

Example 2 can be up-scaled in order to produce the polypeptide at industrial scale.

### Example 3: Proof of Concept In Vitro and in Non-Human Mammals

The present invention is, in part, based on experiments with two animal models of skin ulcers. In these models skin ulcers are induced in diabetic mice by circular biopsy punch or by cycles of pressure loading, and the polypeptide of the invention is topically applied.

Reported herein is a study of administration of the polypeptide of SEQ ID NO: 4 to non-human animals. The polypeptide of SEQ ID NO: 4 is obtainable at high purity by expression as described in Example 1 and purification as described in Example 2.

The aim of this Example is to investigate the efficacy of the topic application of the polypeptide of SEQ ID NO: 4 on wound healing in diabetic mice, related histopathology, pain threshold and plasma human NGF (hNGF) levels. Reference compounds (human NGF (SEQ ID NO: 2), and murine NGF, amino acid sequence available in public resources) are also included in the study.

The animals to which the polypeptide of SEQ ID NO: 4 is administered are characterized by a skin disorder as described herein. The animals represent an animal model of a human who suffers from diabetes mellitus or has a predisposition to suffer from diabetes mellitus, for example diabetes mellitus Type 1 or diabetes mellitus Type 2.

The polypeptide of SEQ ID NO: 4 may be administered in a single dose or in repeated doses. The polypeptide of SEQ ID NO: 4 may be administered to subjects with diabetic ulcers, an animal model for diabetic neuropathic foot ulcers (DFU).

This example includes the following sections:

Example 3A: in vitro PC12 neurite elongation test. The aim of this section was to establish the efficacy of the polypeptide of SEQ ID NO: 4. For this purpose, a conventional in vitro neurite elongation test in NGF-sensitive cells (PC12) was used.

Example 3B: the in vivo efficacy study. The aim of this section was to determine if topical application of the polypeptide of SEQ ID NO: 4 improved wound healing (surgical lesion) in diabetic mice.

The following groups were included:
db/db, intact N=8, each time point
db/db, wound+vehicle N=8, each time
db/db, wound+polypeptide of SEQ ID NO: 4, 1 μg/day; N=8, each time point
db/db, wound+polypeptide of SEQ ID NO: 4, 10 μg/day; N=8, each time point
db/db, wound+polypeptide of SEQ ID NO: 4, 30 μg/day; N=8, each time point
db/db, wound+hNGF, 10 μg/day N=8, each time point
db/db, wound+mNGF, 10 μg/day N=8, each time point
   (dose in μg refers to the respective doses administered per wound (each animal having one wound)

In this section of the study, animals were sacrificed at 7 and 30 days after wound induction, with the following end-points: time-to-closure; histology (N=4) and immunohistochemistry (N=4) of the lesions.

Example 3C: the mechanism: exploratory study. The aim of this section was to explore the molecular mechanisms supporting the positive effect of polypeptide of SEQ ID NO: 4 on wound healing in diabetic mice, focusing on inflammation, extracellular matrix deposition, innervation, angiogenesis.

The following groups were included:
db/db, intact N=6
db/db, wound+vehicle N=6
db/db, wound+polypeptide of SEQ ID NO: 4, 1 μg/day N=6
db/db, wound+polypeptide of SEQ ID NO: 4, 10 μg/day N=6
db/db, wound+polypeptide of SEQ ID NO: 4, 30 μg/day N=6
db/db, wound+hNGF, 10 μg/day N=6
db/db, wound+mNGF, 10 μg/day N=6
   (dose in μg refers to the respective doses administered per wound (each animal having one wound)

In this section of the study, animals were sacrificed at 14 days after wound induction, corresponding to 50% wound healing, with the following end-point: exploration of possible mechanisms responsible for the therapeutic effect of the polypeptide of SEQ ID NO: 4, with regard to the expression and regulation of mRNA (N=252) encoding for proteins involved in the extracellular matrix biology (N=84), angiogenesis (N=84), growth factors and neurotrophins biology (N=84).

Materials and Methods in this Example

### Animals and Monitoring

Mice homozygous for the diabetes spontaneous mutation (Leprdb) (genetic background C57BL/6J) and the respective heterozygote controls from the same colony were used at 8-12 weeks of age (Charles River Laboratories—Calco—Lecco, T/BKS.CG-M+/+LEPR DB/J and S/BKS.CG-M DB/+). See introduction for the group composition and animal sacrifice.

Animals were housed in single cages, with food pellets and water ad libitum, and a dark-light cycle of 12 hours. All animal protocols described herein were carried out according to the European Community Council Directives (2010/63/EU), and approved by the Ministry of Health (n^{o} 350/2015-PB), and comply with the guidelines published in the NIH Guide for the Care and Use of Laboratory Animals.

Glycaemia blood level was measured before treatment, the day after the last treatment and before sacrifice (Contour XT, Bayer, Basel, Switzerland).

Experimental schedule for 8 days cohort:

| | | | | | | | | | | | | |
|---|---|---|---|---|---|---|---|---|---|---|---|---|
| Days | −3 | −2 | −1 | 0 | 1 | 2 | 3 | 4 | 5 | 6 | 7 | 8 |

| | | | | | | | | | | | |
|---|---|---|---|---|---|---|---|---|---|---|---|
| NGF | | | [symbol] | [symbol] | [symbol] | [symbol] | [symbol] | [symbol] | [symbol] | | |
| Treatments | | | | | | | | | | | |
| Photos | | | [symbol] | | [symbol] | | [symbol] | | [symbol] | | |
| Plantar Test | [symbol] | | | | | | | | | [symbol] | |
| Glycaemia | | [symbol] | | | | | | | | | |
| Sacrifice | | | | | | | | | | | [symbol] |

[symbol]

indicates data missing or illegible when filed

Experimental schedule for 30 days cohort:
First week: as for 8 days cohort
Then: photos twice a week until sacrifice
   Glycaemia at day 28
   Sacrifice at day 29

in this example, the experiments are indicated as “8 days”, and “30 days” cohorts, whereas the days of the test or sacrifice, as reported in the experimental schedule, is indicated.

Lesion, medication and monitoring.

A 6-mm diameter circular full-thickness wound was created by a dermal punch biopsy on the midback of the mouse. Briefly, animals were deeply anesthetized by Isofluorane (+2 l/min O2). The skin of the back was shaved with waxing cosmetics and disinfected with Clorexidina 4% (“Clorexyderm” I.C.F. srl Industria Chimica Fine—CR-Italy) or lodopovidone 10% (“Poviderm” Nuova Farmec srl—VR—Italy). A sterile 6-mm diameter punch biopsy tool was used to create full-thickness open wound on the back of the animal. The wound area was immediately covered with the semi-occlusive Tegaderm medication (Tegaderm Roll—3M Health Care, St. Paul, MN, USA) creating a 1.5 cm thick band around the thorax so that mice were not able to gnaw the dressing. A 26-gauge needle was used to infuse 50 μl of medication through the Tegaderm into the wound bed on post wounding days 0-6. Notably, the Tegaderm dressing completely prevented solution leaking from the lesion.

A 6-mm diameter circular full-thickness wound has a surface of 28.26 mm².

Based on that, the doses administered to the animals were as follows:
1 μg dose: 0.0035 μg/mm²
10 μg dose: 0.35 μg/mm²
30 μg dose: 1.05 μg/mm²

Animals were daily monitored for dressing integrity and absence of infections. Tegaderm was weekly changed in all animals, until complete wound healing.

A picture of the wound including a ruler was then taken and the lesion area was measured by computerized image analysis (NIS Elements, Nikon) three time during the first week, then twice a week up to the end of the experiment.

### Administration of the Polypeptide of SEQ ID NO: 4

The polypeptide of SEQ ID NO: 4 was diluted in phosphate buffered saline (PBS) and divided into daily aliquots. All the procedures were performed in ice and final aliquots were stocked at −80° C.

human NGF (hNGF, Recombinant, E. Coli Cat N^{o}: N-245, Alomone, Jerusalem, Israel) and Recombinant mouse NGF (mNGF, Cat N^{o}: 1156-NG, R&D System) were used as control NGF.

Test compounds were daily administered for 7 days starting from the wound induction day. 50 μl of test compounds solution at each concentration were injected under the Tegaderm band on the wound area with a 26-gauge needle. The Tegaderm elasticity allows the sealing of the needle hole after needle retraction, and no leaking of the liquid solution was observed.

### Pain Threshold Monitoring

Thermal hyperalgesia was evaluated in freely moving animals by the Hargreave's' method using the Thermal Plantar Test Instrument (Ugo Basile—Comerio, Varese). Animals were allowed to acclimatize in the plexiglass box of the instrument for 15 min. A constant intensity radiant heat source (beam diameter 0.5 cm and intensity 25 I.R.) was placed under the hind paw and the withdrawal latency (seconds) was recorded as the time from the onset of radiant heat application to paw withdrawal. The mean of four measures was used for statistical analysis. Animals were tested with the Plantar Test on day −3 (before surgery) and day 7 (24 hours after the last application of test compounds).

### Tissue Collection and Processing

At the day of sacrifice, mice were deeply anesthetized (Isoflurane+2 l/min O2) and skin samples (1 cm×1 cm) were taken from the area of the wound. For the study B, in each group 4 samples were collected for immunohistochemistry and 4 samples were collected for histology. For the study C, 6 mm skin area were taken with the excisional punch (wound area), a ring of 8 mm around this (wound perimetral area) and a 6 mm area from intact skin were collected.

Samples collected for histology were embedded in paraffin, sectioned and stained using hematoxylin and eosin (H & E); samples collected for immunohistochemistry were postfixed, washed in sucrose PBS, cryosectioned and processed for indirect immunofluorescence.

### Immunohistochemistry and Quantitative Analysis

Skin was immersed in paraformaldehyde 4% (w/v) and picric acid saturated aqueous solution in Sörensen buffer 0.1 M pH 7 for 24 h, then washed for at least 48 h in 5% sucrose in 0.1 M phosphate buffer. After freezing in CO2, sections (14 μm thick) were cut using a cryostat (HM550 Microm, Bio-Optica). Sections were collected on gelatin coated slides first incubated in 0.1 M PBS at room temperature for 20 min, followed by overnight incubation at 4° C. in a humid atmosphere with the primary antibodies diluted in 0.3% PBS-Triton X-100, v/v. The following antisera were used in this study: Laminin (Rabbit, Sigma, 1:1000); protein gene product 9.5 (PGP-9.5) (Rabbit, Boheringer, 1:2000). After rinsing in PBS for 20 min (2×10 min), the sections were incubated at 37° C. for 30 min in a humid atmosphere with the secondary antisera conjugated with Rhodamine Red™-X-conjugated—affinity-pure Donkey anti-Rabbit IgG (Jackson Immunoresearch) diluted in PBS triton 0.3%. Sections were then rinsed in PBS (as above) and mounted in glycerol containing 1,4-phenylendiamine (0.1 g/l).

Immunohistological images were captured by a Nikon Eclipse E600 microscope equipped with digital CCD camera Q Imaging Retiga-2000RV (Q Imaging, Surrey, BC, Canada). Analysis were performed using the Nis-Elements AR 3.2 software. The Laminin and PGP9.5 immunoreactive area was calculated as a fraction (percentage of) in the epidermic layer, at 7 days and 30 days after the induction of the skin lesion. The sprouting index was estimated by observing the number of sections in which the PGP9, 5 IR was approaching the ulcer border. For all the morphological analysis, five images were analysed for each animal and two levels/animal. All analyses were performed in blinded manner. The mean value/animal was used for the statistical analysis.

hNGF Quantification

Blood was collected in EDTA-K2 Vacuntainer tubes and within 30 min it was centrifuged at 3000×g for 10 min at 4° C. The plasma was collected, aliquoted in polypropylene tubes and stored at −80° C. until used.

The kit Human Adipokine Magnetic Bead Panel 2 (HADK2MAG-61K, EMD Millipore Corporation, Billerica, MA, USA) was used to quantify hNGF in plasma samples using xMAP technology and a MAGPIX Luminex platform. This technology is based on the use of different populations of color-coded beads conjugated with monoclonal antibodies specific to a particular protein, thus allowing simultaneous capture and detection of specific analytes with high sensitivity from a small volume of sample. We have used a simple version of the kit including only one population of beads conjugated with human NGF-β monoclonal antibody. The assay was performed following the manufacture's specifications with minor modifications.

In brief, after the incubation of the specific human NGF-β monoclonal antibody conjugated beads population with plasma samples (25 μl) overnight at RT, beads were washed and incubated first with detection antibody solution for 1 h at RT, then with the streptavidin-phycoerythrin conjugated solution for 30 min at RT. After washing, beads were resuspended in 100 μl of Drive fluid and read on the MAGPIX instrument. Data were analyzed with xPONENT 4.2® software and results were expressed as pg/mL. We obtained values within the dynamic range of the standard curve (from 10000 to 0,128 μg/mL) for all the samples. Standard curves had a correlation coefficient (R2) value>0.98. The accuracy of the obtained results was further verified through the values obtained for the quality control solutions included in the kit (QC1 and QC2), which were within the range specified from kit's manufactures. Detection limit of human NGF-β was 0.3-0.7 μg/mL.

This assay was chosen because of the high sensitivity and specificity for hNGF, compared to other ELISA methods.

### PC12 Culture and Treatments

Cells were maintained in standard culture condition in the culture medium (DMEM, Horse serum 10%, FBS 5% and pen/strep 1×) in T25 cm2 flasks (NUNC). After at least two passages, cells were seeded (1000 cells/well) on cell culture treated 96 wells flat-bottom cell plates (NUNC). After 1 day in vitro (DIV), culture medium was removed and cells were maintained in the deprivation medium (DMEM, Horse serum 1%, FBS 0.5% and pen/strep 1×). Cells were treated with three different concentrations of all the test compounds (50, 100 and 200 nM), 24 hours after the serum deprivation. After 2 DIVs medium was refreshed and at DIV 7, cells were fixed and stained for beta-III-tubulin antigen by using indirect immunofluorescence (FIG. 1).

### Immunocytochemistry

At 7 DIV cells were fixed with cold Paraformaldehyde 4% for 20 minutes. After the treatment for 1 hour with the blocking solution (PBS, triton X-100 0.3%, BSA 1% and Normal Serum Donkey 1%) cells were incubated with the primary antisera (Mouse Anti-beta-III-tubulin, 1:1000; R&D) overnight at 4° C. Cells were then incubated with the secondary Anti-mouse antibody (Donkey Anti-mouse Alexa-488 conjugated; 1:500; Jackson) at 37° C. for 30 minutes. Finally, cells were incubated with the nuclear dye Hoechst33258 at RT for 20 minutes.

### Cell-Based High Content Screening Analysis

Analysis of neurite elongation was performed with Cell Insight™ CX5 High Content Screening (HCS; Thermo Scientific), using the Neuronal Profiling BioApplication. The software is able to recognize every single cell in each well, by the presence of the nuclear dye fluorescence. Each nucleus is identified as an object, and every object corresponds to a single cell. The system recognizes the green fluorescence (beta-III-tubulin immunoreactivity) around the nucleus identifying the cell body. The Neuronal Profiling tool is able to recognize and track all the neuritis emerging from each cell body. This allows to count and measure all the neuritis from every cell. Cells aggregate were not recognized as a single cell dimension object and were excluded from the analysis.

A number of 2000-4000 single cells/well and 6 wells/treatment were analysed.

### RT-PCR

The exploratory study on the possible mechanisms supporting the positive effect of the polypeptide of SEQ ID NO: 4 on wound healing in diabetic mice was performed using an exploratory strategy (pathway-focused gene expression analysis using the RT2 Profiler PCR Arrays) and focusing on the main molecular pathways involved in wound healing at 50% of the repair process, e.g. angiogenesis, extracellular matrix and adhesion proteins, growth factors. Samples were collected from the core of the lesion (6 mm diameter) and RNA was extracted from all animals (6 animals per group), quantified (Nanodrop 2000 spectrophotometer) and pooled (100 ng per animal). Thus, 600 ng of RNA per group was used for the reverse transcription.

A single PCR array was performed for each group, using the CFX96 real time PCR instrument (BioRad). For all the plates was used the same threshold and the relative expression of gene was calculated by the 2-ΔΔCq comparative method. The mouse angiogenesis, extracellular matrix and adhesion protein (ECM), and the growth factors (GFs) Rt2 Profiler™ arrays (QIAGEN) were used to profile the expression of 250 key genes involved in angiogenesis, ECM, and GFs (84 genes each) with cDNA synthesised using RT2 First Strand kit (QiAGEN) according to the manufacturer's instruction.

### Results

The results are presented in the following order:
PC12 in vitro assay
Efficacy, 8 days
Efficacy, 30 days
Mechanism, 14 days

### Example 3a: In Vitro PC12 Neurite Elongation Test

The efficacy of the polypeptide of SEQ ID NO: 4 was tested in vitro on PC12 cells (see FIG. 1 for the experimental design), measuring the neurite elongation by means of cell-based high content screening, using the following parameters:
Mean Neurite Average Length: represents the average neurite length per cell;
Mean Neurite Total Length: represents the total neurite length per cell;
% High Neurite Maximum Length: represents the percentage of cells showing a neurite equal or longest the cell body length.

First, all the test compounds doses where analysed and compared to the vehicle group (data not shown). In this report, only the results for more effective dose of each test compound have been shown (FIG. 1). Cells exposed to all the test compounds effective doses showed an increase in the mean neurite average length (A; mNGF, P=0.0394; hNGF, P=0.0196; polypeptide of SEQ ID NO: 4, P=0.0033) and mean neurite total length (B; mNGF, P=0.0338; hNGF, P=0.0006; polypeptide of SEQ ID NO: 4, P=0.0211; Aloe, P<0.0001) in comparison to the vehicle treated cells. Only the polypeptide of SEQ ID NO: 4 (P=0.0367) was able to increase the percentage of cells showing long neuritis.

### Example 3B: The Efficacy Study, 8 Days Cohort

### Animal Monitoring

Animals were monitored for glucose blood levels before wound generation. Results are reported in FIG. 3. Glycaemia was higher in diabetic mice than in control animals, as measured in the pilot study. No differences were observed among the animals assigned to the different experimental groups.

The body weight gain between day 0 and day 7 post-lesion is reported in FIG. 4. No differences were observed either according the time (day 0 vs day 7) or the treatment.

### Hyperalgesia

Thermal hyperalgesia was evaluated in freely moving animals by the Hargreave's Method using the Thermal Plantar Test Instrument at day −3 (before skin lesion) and the day after the last test compound application (day 7). Results are illustrated in FIG. 5. No differences among groups were observed at day 0. By comparing mean paw withdrawal latency at day 0 and day 7 in the same treatments group, a significant reduction was observed in hNGF-treated animals at day 7. No hyperalgesia to thermal stimuli was observed in the other groups. Notably, a higher threshold was observed in polypeptide of SEQ ID NO: 4-treated mice at day 7, indicating a higher pain threshold in this group.

### Time-to-Closure

Wound healing evaluation was performed by macroscopic observation of photos, that were taken starting from day 0 (surgery day) every two days during the first week. “Time-to closure” was measured on these wound images using NIS-elements (Nikon). Results are reported in FIG. 6, where both the external and internal areas are plotted. Two-ways ANOVA indicate a time effect (F (4,203)=41.25, p<0.0001) and a group effect (F (5,203)=2.565, P=0.0282).

Time-to-closure at day 7 is reported in FIG. 7. No differences are observed among groups, in spite of the fact that a dose-dependent trend to promote wound healing is observed in the polypeptide of SEQ ID NO: 4-treated groups.

### NGF Plasma Levels

Blood was collected at sacrifice, thus 48 hours after the last application of test compounds. NGF plasma levels were determined by an antibody-based assay using an antibody that is capable of detecting human NGF, murine NGF and also the polypeptide according to SEQ ID NO: 4. Herein, the so-determined NGF plasma levels are referred to as “total NGF plasma levels”. Results are reported in FIG. 8. A dose-dependent increase in total NGF plasma levels was observed in treated mice, reaching values higher than 350 μg/ml. Moreover, an increase was also observed in the mNGF-treated group. This is not surprising, since the recombinant mouse NGF-β used for treatments is a homodimer of two amino acid polypeptides that shares around 90% identity at amino acid level with human NGF, and is recognized by the same antibody.

### Example 3C: The Efficacy Study, 30 Days Cohort

### Animal Monitoring

Animals were monitored for glucose blood levels before wound generation, after the end of test compounds administration and at sacrifice. Results are reported in FIG. 9. We observed a progressive increase in glucose blood levels over the 28 days of observation in all the experimental groups. No differences among the treatments groups were observed at the different times.

The body weight gain between day 0 and day 28 post-lesion is reported in FIG. 10. No differences were observed either according the time or the treatment.

### Hyperalgesia

Thermal hyperalgesia was evaluated in freely moving animals by the Hargreave's method using the Thermal Plantar Test Instrument at day −3 (before skin lesion) and at day 7, 24 hours after the last NGF application. Results are illustrated in FIG. 11. No differences among groups were observed at day 0. By comparing the latency at day 0 and day 7 in the same treatments group, a non-significant trend to a reduction in paw withdrawal latency was observed at day 7 in hNGF-treated animals. However, when the data from 8 days and 30 days cohorts were pooled, a significant reduction of pain threshold was observed in hNGF-treated animals, thus suggesting that this hNGF formulation induces hyperalgesia. No differences were observed in the other groups. Results are presented in FIG. 12.

### Time-to-Closure

Wound healing evaluation was performed by macroscopic observation taking photos every two days during the first week starting from day −3 (surgery day), then twice a week until sacrifice. “Time-to closure” was measured on these wound images using NIS-elements (Nikon).

Individual data for “Time-to-closure” are presented in tabular form (FIG. 13). Serial external wound area measurements are plotted against time in FIG. 14. Two-ways ANOVA indicate a time effect (F (50,434)=417.3, p<0.0001), a treatment effect (F (5,434)=21.45, p<0.0001), and an interaction between treatment and time (F (50,434)=1.638, p=0.0055).

In mice treated with the polypeptide of SEQ ID NO: 4, the rate of wound healing was significantly accelerated compared to vehicle treated animals in a dose-dependent manner.

Time-to-closure at day 8 is reported in FIG. 15, where data from both 8 days and 30 days cohorts are pooled. A significant reduction of wound healing already at this time is observed in the group treated with the polypeptide of SEQ ID NO: 4 at the dose of 30 μg/day, compared to vehicle treated group.

### NGF Plasma Levels

Blood was collected at sacrifice. NGF plasma levels were determined by an antibody-based assay using an antibody that is capable of detecting human NGF, murine NGF and also the polypeptide according to SEQ ID NO: 4. Herein, the so-determined NGF plasma levels are referred to as “total NGF plasma levels”. Results are reported in FIG. 16. Total NGF plasma level are very low (compared to FIG. 8), not higher than 10 μg/ml, and similar in all groups.

### Histology (at 8 and 30 Days) (See Also the Report of the Pilot Study)

Samples of the skin containing the ulcer's area including a 5 mm margin of intact skin were excised down, embedded in paraffin and serially sectioned according to the schema presented in FIG. 17B. Sections were then stained (H&E) and representative low-power images at the different levels of the wound are reported in FIG. 17A. High-power micrographs illustrate the re-epithelization process at the wound border (FIG. 17D), where the epidermis migrating tongue (MET) is evident, the extensive granulation tissue in the derma below the epidermis layer, characterized by inflammation, cell proliferation, matrix deposition (FIG. 17E) and angiogenesis (FIG. 17F). Re-epithelization has been evaluated by measuring the epidermis layer thickness. Representative images from intact animals, vehicle-, polypeptide of SEQ ID NO: 4, 1 μg/day, polypeptide of SEQ ID NO: 4 30 μg/day-treated mice are presented in FIG. 18. The polypeptide of SEQ ID NO: 4 induces a dose-dependent thickening of the epidermis layer, which appears much higher that in the intact skin. The basal layer of the epidermis is characterized by hypercellularity of the basal and spinous layers, possibly reflecting an increased cell proliferation. Moreover, the dermis is also thicker and strongly stained, suggesting a higher extracellular matrix deposition, and is enriched by skin annexes (glands and hair follicles) also according to the dose. Epidermis thickness in all groups is presented in the graph. The polypeptide of SEQ ID NO: 4 induces a dose-dependent thickening of the epidermal layer, that grows much thicker than in the vehicle group. Also mNGF and hNGF induce the same effect, comparable to the dose-matching group treated with the polypeptide of SEQ ID NO: 4.

### Immunohistochemistry (at 8 and 30 Days)

Skin reinnervation was analysed through the immunostaining for the protein PGP9.5, a highly sensitive neuroectodermal marker extensively used to visualized cutaneous innervation. The anatomy of the skin innervation is presented in FIG. 19, where PGP9.5-IR fibres in the intact mouse skin are visualized. In particular, the subcutaneous, deep cutaneous and sub-epidermal plexi are visualized; the sub-epidermal plexus provides the epidermal free nerve endings to the epidermis.

The effect of topical application of the polypeptide of SEQ ID NO: 4 on nerve re-growth in the repaired skin has been analysed using a “spouting index” at the lesion border at day 8 after lesion, and PGP9.5-IR in the epidermis and derma of the repaired area. Representative images are reported in FIG. 20. Panels A, B and C illustrates PGP9.5-IR at 30 days, in intact animals, vehicle, and the polypeptide of SEQ ID NO: 4 30 μg/day-treated mice, respectively. Results from the morphometric analysis are presented in FIG. 21. 30 μg/day application of the polypeptide of SEQ ID NO: 4 induces a significant increase in sprouting at 8 days, such as hNGF. At 30 days, while in vehicle-treated animals the innervation is not yet restored, no differences between intact and NGF-treated animals were observed administering the polypeptide of SEQ ID NO: 4 (all dosages) and mNGF. On the contrary, and hyperinnervation is observed using hNGF.

The effect of topical application of the polypeptide of SEQ ID NO: 4 on angiogenesis was estimated using laminin as marker. Laminin is a basal membrane marker, thus labelling several structures in the skin, including endothelial cells. Other endothelial markers like PECAM (also known as CD31), Von Willebrand factor, collagen, provided staining not suitable for quantification in the fixation conditions used in this study. Representative images are reported in FIG. 22. Panels A illustrate the epidermal layer as visualized by conventional histology (H&E). Arrows indicates the basal layer. Panel B illustrates the basal membrane underlying the epidermis (arrows); Panel C illustrates the sensory innervation of the epidermis deriving from the subependimal plexus; Panel D illustrates the ulcer border and related innervation at 8 days (E) and after skin repair (F). Panels G-1 illustrates angiogenesis at 8 days (G, EE; H, laminin-tR) and 30 days (I)

Results from the morphometric analysis are presented in FIG. 23. Application of the polypeptide of SEQ ID NO: 4 at 30 μg/day induces a significant increase in laminin-IR at 8 days, such as hNGF, which is still present at 30 days, possible reflecting undergoing angiogenesis. 5.

### The Mechanism: Exploratory Study

### Gene Expression Regulation

The aim of the study was to explore molecular mechanisms supporting the positive effect of the polypeptide of SEQ ID NO: 4 on wound healing in diabetic mice, focusing on inflammation, extracellular matrix deposition, innervation, angiogenesis. An exploratory strategy (pathway-focused gene expression analysis using the RT2 Profiler PCR Arrays has been used to identify the main molecular pathway in the wound at 50% of the repair process. The mouse angiogenesis, extracellular matrix and adhesion protein (ECM), and the growth factors (GFs) Rt2 Profiler™ were used to profile the expression of 252 key genes involved in angiogenesis, ECM, and GFs (84 genes each).

Expression analysis for each panel (angiogenesis, ECM, growth factor) is presented by:
the gene list;
the heat map providing a graphical representation of fold regulation expression data between two groups overlaid onto the PCR array plate layout;
the scatter plot comparing the normalized expression of every gene on the array between two groups by plotting them against one another to quickly visualize large gene expression changes, and the list of genes whose expression changes are grater than the selected boundary (≥3).

The scatter plots illustrate the group comparison as follow:
db/db vs. WT mice (WT as control group)
db/db vehicle vs. db/db intact (db/db intact as control group)
db/db NGF (polypeptide of SEQ ID NO:, mNGF, hNGF) vs db/db vehicle (db/db vehicle as control group)
db/db NGF (polypeptide of SEQ ID NO:, mNGF, hNGF) vs db/db intact (db/db intact as control group)

Results are evaluated as Extracellular matrix and adhesion molecules and Growth factors and neurotrophins (figures not shown).

The main results and conclusions, from the present analysis, are the following:

### Genotype Effect:

The comparison between db/db intact vs WT intact indicates that numerous angiogenesis and ECM genes are differentially regulated according to the genotypes, while very few GF gene are differentially expressed, thus suggesting that ECM and angiogenesis are the processes mainly affected by the diabetic condition for wound repair.
Lesion effect in db/db:
The lesion induced the down-regulation of numerous angiogenesis and ECM genes, and the up-regulation of few GFs genes, thus suggesting than ECM and angiogenesis are the main processes driving wound repair also in diabetic mice.
polypeptide of SEQ ID NO: 4 effect in db/db (vs vehicle):
the polypeptide of SEQ ID NO: 4 down-regulates several genes, including: Angiogenesis: akt, Ccl2 (chemokine (C-C motif) ligand 2), Ctgf (connective tissue growth factor), Hif1a, MMP14, thbs2 (thrombospondin 2);
the polypeptide of SEQ ID NO: 4 up-regulates several genes, and just some of then are also regulated by hNGF and mNGF
the polypeptide of SEQ ID NO: 4 does not regulate GF genes, some of them are regulated by hNGF and mNGF

A STRING analysis was also performed (data not shown). STRING is a biological database and web resource of known and predicted protein-protein interactions, that is extensively used to search interaction relationships among differentially expressed genes. “Clustering” analysis by STRING software is based on all genes that are regulated in the different arrays.

### CONCLUSIONS

The main conclusions from this example are the followings:
1. PC12 cell coupled to HCS as analytical approach is a suitable approach to evaluate the in vitro efficacy of the polypeptide of SEQ ID NO: 4;
2. the polypeptide of SEQ ID NO: 4 improves would healing in a dose-dependent manner.
3. The polypeptide of SEQ ID NO: 4 strongly increases epidermal layer repair and induces a strong thickness increase; the polypeptide of SEQ ID NO: 4 also positively affects re-innervation and angiogenesis (as evaluated by laminin-IR). All these parameters in polypeptide of SEQ ID NO: 4-treated mice are higher than in control, intact mice, thus suggesting that the re-modelling phase of wound healing must be evaluated in a further study.
4. The exploratory study suggests that AKT-mTOR pathway might be involved in the effect of polypeptide of SEQ ID NO: 4. Akt and mTOR are considered survival and cellular growth promoters, and it has been suggested that the transient pharmacologic activation of the PI3K-Akt-mTOR signaling axis may represent a novel clinical intervention strategy to accelerate the healing. Notably, an impaired AKT-mTOR pathway has been indicated as possible cause of wound healing impairment in diabetic mice, and AKT-mTOR pathway has been demonstrated to be involved in improved wound healing by several molecules, like Notoginsenoside Ft1 in diabetic mice; acemannan; SR-0379; microRNA-99 family.

Thus, the polypeptide of SEQ ID NO: 4 is a recombinant protein with a polypeptide sequence similar to human nerve growth factor, but with at least one mutation that renders it painless (hNGFp) and therapeutically effective.

| | | | | | | | |
|---|---|---|---|---|---|---|---|
| Day 0 | Day 1 | Day 2 | | | | | |
| PM | PM | AM | Day 3 | Day 4 | Day 5 | Day 6 | Day 7 |

| | | | | |
|---|---|---|---|---|
| PC12 | Serum | NGF | Medium | ICC |
| seeding | deprivation | | | |

Table above: Experimental design for in vitro neurite elongation test in PC12 cells. See methods for further details

### Example 4: Proof of Concept: Pressure Ulcer Model in Mice

Reported herein is a study of administration of the polypeptide of SEQ ID NO: 4 to non-human animals. The polypeptide of SEQ ID NO: 4 is obtainable at high purity by expression as described in Example 1 and purification as described in Example 2.

### Methods

Control (C57BL6, albino) and genetically diabetic C57BL/KsJ-m+/+Leprdb (db/db) male mice, Jackson laboratories, 8-10 weeks old were included in the experiment. Under gaseous anesthesia the animals were shaved on the back and the shaved area was thoroughly cleaned to prevent skin irritation. A skin fold was raised and two magnetic ceramic disks of 12 mm diameter and a thickness of 5.0 mm, with an average weight of 2.4 g and 1000 G magnetic force (Magnetic Fountain, Castle Rock, CO), were applied to the skin leaving a “bridge” of approximately 5.0-mm of skin between the two magnets. This process creates 50 mm Hg of compressive pressure between the two plates, as has been documented to be necessary to cause local tissue ischemia (Peirce et al., 2000, Wound Repair Regen., vol. 8, p. 68-76.). Three cycles of ischemia-reperfusion (I/R) were applied to induce the formation of 2 ulcers of homogeneous severity. A single cycle of I/R consists of a period of 12 h for the application of the magnets starting at 8:00 a.m., followed by a rest period of 12 hours without magnets. Treatments with vehicle and test compounds was started 3 days after the end of the 1/R cycles to enable the surgical curettage of the ulcers consisting of the removal of fibrin exudate and necrotic tissue.

Some of the mice were subjected to an investigative treatment with the polypeptide of SEQ ID NO: 4, which may also be referred to as recombinant human mutant nerve growth factor painless (hNGFp). In detail, the following experimental groups were investigated:
db/db, vehicle
db/db, the polypeptide of SEQ ID NO: 4, 1 μg/cm²/day
db/db, the polypeptide of SEQ ID NO: 4, 10 μg/cm²/day
db/db, the polypeptide of SEQ ID NO: 4, 100 μg/cm²/day.

Treatment was continued daily for 14 consecutive days and then twice weekly until closure at the same dosage (dosage calculated with respect to ulcer size at the time point of treatment). The ulcers were monitored by visual inspection to establish the day of closure. Moreover, a picture of the wound including a ruler was taken and the lesion area measured by computerized image analysis when the ulcers were medicated twice a week. The pressure ulcer assessment was performed according to a standardized scale and by measuring the wound area by computerized image-analysis.

The effect of the compound on pain threshold was evaluated in freely moving animals at the site of injury with the Bioseb's electronic von Frey, an electronic apparatus that allows the determination of mechanical pain sensitivity threshold in rodents.

The histology of the lesion, the innervation and angiogenesis will be analysed by histology and immunohistochemistry and computerized image analysis.

### Results

Placing the magnets on a skin fold on the backs of diabetic mice induced irreversible damage involving the entire dermo-epidermal tissue under the site of compression. Both the visual inspection (presence of a necrotic and hemorrhagic area) and the histological analysis confirmed that the 3 l/R cycles were able to induce lesions that were reminiscent of a pressure ulcer.

In all the groups treated with the polypeptide of SEQ ID NO: 4 the rate of wound healing was accelerated compared to vehicle-treated animals. The closure of the ulcers in the polypeptide of SEQ ID NO: 4-treated animals was apparent starting from days 17 and 21, whereas vehicle-treated animals underwent healing starting from day 23.

At day 28, the last day of observation, in more than 80% of the polypeptide of SEQ ID NO: 4-treated animals the cutaneous ulcers were completely closed; as shown in FIG. 25; compared to vehicle-treated animals, the effect was statistically significant at all doses of the polypeptide of SEQ ID NO: 4, being the probability of healing in the former group less than 60%. These data are in line with literature evidence showing that in animal models of diabetes the rate of wound healing is impaired and, together with data generated in the surgical ulcer model (Example 3), they suggest that the polypeptide of SEQ ID NO: 4 may normalize the delayed healing process in diabetic mice.

In addition to the positive effects on wound healing, histological and immunohistochemistry data provide further evidence that the polypeptide of SEQ ID NO: 4 has the biological ability to improve the degree of the parameters of wound healing in healing-impaired diabetic mice. At the histological level, in the repaired area a complete reepithelization and the restoration of a normal skin anatomy were observed after treatment with the polypeptide of SEQ ID NO: 4. Immunohistochemistry showed that the polypeptide of SEQ ID NO: 4 also positively affected reinnervation (as measured by PGP 9.5 immunoreactivity in the epidermidis) and neo-angiogenesis (as measured by PECAM immunoreactivity in the derma) (FIG. 26 A and B).

Since wild-type NGF has been reported to increase pain sensitivity at the site of administration, the pain mechanical threshold was evaluated after 14 consecutive days of treatment with the polypeptide of SEQ ID NO: 4 by applying a mechanical stimulus at the border of the ulcers. No modifications of the pain mechanical threshold were observed in comparison with vehicle-treated diabetic mice suggesting that the polypeptide of SEQ ID NO: 4 after chronic topical treatment can exert its positive trophic effects on skin in a large interval of doses without causing nociceptors sensitization (FIG. 27).

### Example 5: A Randomised, Double-Blind, Placebo-Controlled Study to Investigate the Safety, Tolerability, Pharmacokinetic and Pharmacodynamic Profiles of the Polypeptide of SEQ ID NO: 4 after Single and Repeated Ascending Doses in Subjects with Diabetic Neuropathic Foot Ulcers (DFU)

Reported herein is a study of administration of the polypeptide of SEQ ID NO: 4 in single and repeated ascending doses in participants with diabetic neuropathic foot ulcers (DFU). The participants are human subjects.

The trial described in this example has received ethical approval from the respective authorities.

The polypeptide of SEQ ID NO: 4 may also be referred to as recombinant human mutant nerve growth factor painless (hNGFp). The polypeptide of SEQ ID NO: 4 is alternatively termed “RECOMBINANT HUMAN NERVE GROWTH FACTOR (RHNGF)” or “SUB77552”, and the full molecular formula is C580H895N1630176S8. The polypeptide of SEQ ID NO: 4 is obtainable from biological/biotechnological origin (other than Advanced Therapy IMP (ATIMP). It is a recombinant medicinal product (see also Example 1). More particularly, the polypeptide of SEQ ID NO: 4 is obtainable by expression as described in Example 1 and purification as described in Example 2. In particular, the high purity obtainable as described in Example 2, preferably under GMP standards, enables the use of the polypeptide of SEQ ID NO: 4 as a medicament.

As used in this example, the polypeptide of SEQ ID NO: 4 is an Investigational Medicinal Product (IMP). According to Directive 2001/20/EC an “IMP” is a “a pharmaceutical form of an active substance or placebo being tested or used as a reference in a clinical trial, including products already with a marketing authorization but used or assembled (formulated or packaged) in a way different from the authorised form, or when used for an unauthorised indication, or when used to gain further information about the authorised form.” Herein, the polypeptide of SEQ ID NO: 4 is an IMP to be used in a first-in-human clinical trial. Thus, this example describes a first-in-human clinical trial. No risk factors according to the guidance first-in-human have been identified.

The IMP used in this Example, is provided as a clear, colourless solution of hNGFp prepared at 1 mg/ml of the polypeptide of SEQ ID NO: 4. The polypeptide of SEQ ID NO: 4 is adjusted to the desired concentration and filled in a glass vial. The concentration of the solution is 1 mg/ml.

The polypeptide of SEQ ID NO: 4 is administered to human subjects in need thereof. Said polypeptide of SEQ ID NO: 4 is administered as cutaneous solution. This is not a specific pediatric formulation.

The human subjects in need of administration the polypeptide of SEQ ID NO: 4 are subjects with diabetic neuropathic foot ulcers (DFU). No risk factors according to the first in human guidance have been identified.

The polypeptide of SEQ ID NO: 4 is administered topically. Thus, the polypeptide of SEQ ID NO: 4 is for topical use (noncurrent).

The polypeptide of SEQ ID NO: 4 is administered at a total dose of 0.3 to 6 μg/mm². “mm²n” refers to the area of the ulcer. The indicated amount (in μg) refers to the amount of the polypeptide that is administered per day.

The polypeptide of SEQ ID NO: 4 is administered twice daily for 14 consecutive days. After that, administration is discontinued.

In this study there is a placebo. The placebo is referred to as PL1. The placebo is a cutaneous solution. The placebo is for topical use (noncurrent). The placebo is a placebo for the polypeptide of SEQ ID NO: 4 (PR1). The placebo is otherwise identical to the IMP (PR1). Placebo is administered identical to the polypeptide of SEQ ID NO: 4.

PR1 and placebo 1 are both prepared for the Trial at and by Klifo A/S, Smedeland 36, 2600 Glostrup, Denmark.

There is no other (comparative) medicinal product in this study.

The subjects being subjected to this study suffer from or afflicted with or predisposed for a disease, which is a skin and connective tissue disease. More particularly, the subjects being subjected to this study are subjects having Diabetic Neuropathic Foot Ulcers (DFU). Diabetic Foot Ulcer is a major complication of diabetes mellitus, a non-healing or poorly healing full-thickness wound, through the dermis, below the ankle in an individual with diabetes.

MedDRA version, system organ class, level, term and classification
code (EMEA EudraCT website (http://eudract.ema.europa.eu/)):

| Version | System Organ Class | Classification Code | Term | Level |
|---|---|---|---|---|
| 20.0 | 100000004858 | 10012664 | Diabetic foot ulcer | LLT |

The Trial has an independent data monitoring committee.

The Initial estimate of the duration of the trial is 2 years, 1 month.

The planned number of subjects included in 92 (60 of which in the age range 18-64 years; 32 of which in the age range of 65 years or more). The group of trial subjects consists of patients and does not comprise healthy volunteers. Specific vulnerable populations are included.

The treatment or care after the subject has ended his/her participation in the trial is the standard of care.

Ethics approval from the authorities has been obtained. A favorable opinion has been issued.

### Objective of the Trial:

Main objective: To assess the safety and tolerability of single and multiple days' topical dosing with the polypeptide of SEQ ID NO: 4 in subjects with DFU.

Secondary objectives:
(a) To assess the pharmacokinetic profile of systemically available drug following single and multiple days' topical dosing with the polypeptide of SEQ ID NO: 4 in subjects with DFU;
(b) To assess the pharmacodynamic effects of multiple days' topical dosing with the polypeptide of SEQ ID NO: 4 on the healing of DFU over a 12-week period.

There is no sub-study.

### Principal Inclusion Criteria:

Part 1 SD and Part 2 MD:

Subjects must meet all the following criteria in order to be eligible for enrolment into the study:
1. Subject's written informed consent obtained prior to any study related procedure;
2. Male or female subject, aged 18-80 years (extremes inclusive), diagnosed with Type I or Type II diabetes mellitus, with glycosylated haemoglobin (HbA1c)≤10%.
3. Female subjects of non-childbearing potential (WONCBP): —they must report surgical sterilization (performed at least 6 months prior to screening), or—menopause (must have had no regular menstrual bleeding for at least one year prior to screening, age≥45 years and FSH at screening≥40 mIU/ml).
4. Female subject with childbearing potential (WOCBP): they must be using one or more of the following reliable methods of contraception during the study period and at least within 90 days after the last study drug administration: a) Placement of an intrauterine device (IUD) or intrauterine system (IUS). b) Hormonal contraception (implantable, patch, oral). c) Barrier methods of contraception: condom or occlusive cap (diaphragm or cervical vaults/caps) with spermicidal foam/gel/film/cream/suppository. d) Male Partner sterilization (with the appropriate postvasectomy documentation of the absence of sperm in the ejaculate);
5. Male subjects; they must be using two effective methods of contraception during the entire study period and not donate sperm within 90 days after the last study drug administration.
6. Presence of at least one diabetic foot ulcer meeting the following criteria:
a) Diagnosed as a full-thickness, neuropathic DFU, located at or distal to the malleolus (excluding ulcers between the toes but including those of the heel).
b) SD: Present for 6 weeks to 12 months, and of 3-5 cm2 in area following sharp debridement, confirmed at screening.

MD: Present for 6 weeks to 12 months, and of 3-5 cm² in area following sharp debridement, confirmed after the 2 weeks run-in period.
c) A minimum 2 cm margin between the qualifying study ulcer and any other ulcers on the specified foot.
d) Depth≥5 mm and graded 1A according to “The University of Texas Staging System for Diabetic Foot Ulcers” (22), with no capsule, tendon or bone exposed and no tunnelling, undermining, or sinus tracts, after the initial sharp debridement.
7. Subject must be able to hold the target ulcer in such a position and orientation that the study medication can be applied without significant loss of substance through run-off, until the dressing has been applied.
8. Adequate vascular perfusion of the affected limb demonstrated within 30 days prior to screening, as defined by at least one of the following:
a) Ankle-Brachial Index (ABI)≥0.9 and ≤1.2, confirmed by transcutaneous oxygen partial pressure (TcPO2)>50 mmHg
b) Toe pressure (plethysmography)>50 mmHg
c) Doppler ultrasound (biphasic or triphasic waveforms) at least on two vessels at the ankle consistent with adequate blood flow to the affected extremity, as determined by SoC.

### Principal Exclusion Criteria:

Part 1 SD and Part 2 MD:
Subjects must meet none of the following criteria in order to be eligible for enrolment into the study:

1. For females only: pregnant or lactating female subject, confirmed by a positive serum pregnancy test at screening and a urine test performed on Day-1.

2. Subject with:
a) Ulcer(s) accompanied by infected cellulitis, osteomyelitis, or clinical signs or symptoms of infection according to the Infectious Diseases Society of the America's Guidelines (IDSA) (19).
b) Gangrene or necrosis on any part of the affected limb.
c) Active or chronic Charcot's foot on the study limb.
d) Planned vascular surgery, angioplasty or thrombolysis or revascularization procedure performed within 1 month prior to enrolment.
e) Ulcers involving exposure of tendon, bone, or joint capsule (It is acceptable to have ulcers extending through the dermis and into subcutaneous tissue with presence of granulation tissue).
f) Ulcer(s) of non-diabetic aetiology.
g) Previous major amputations on the same target foot.
h) Actual or recent (3 weeks) antibiotic therapy for any reason.
i) Bedridden subjects or subjects with a life expectancy less than one year.

3. Use of any other growth factor therapy in the 6 months prior to screening.

4. History of malignancy in the 5 years prior to screening or those with a strong family history of cancer (e.g. familial cancer disorders), with the exception of squamous cell or basal cell carcinoma of the skin that has been definitively treated.

5. Clinically significant cardiovascular, pulmonary, renal, endocrine, hepatic, neurological, psychiatric, immunological, gastrointestinal, haematological or metabolic disease that is, in the opinion of the Investigator, not stabilised or may otherwise impact subject safety or study results (in cases of doubt, the Sponsor's Clinical Research Physician should be consulted).

6. Subject undergoing haemodialysis or peritoneal dialysis or with chronic renal insufficiency (plasma creatinine>2 mg/dl).

7. Subject with significantly abnormal key laboratory parameters interfering with the safety of the patient according to the PI judgement.

### Scope of the Trial/Parts of the Trial:

The Trial has two parts:
Part 1 SD—single ascending dose
Part 2 MD—multiple ascending dose

### Dosages:

SD: 0.3, 1, 3 and 6 μg/mm²
MD: 1 and 3 μg/mm²

### End Points:

### Primary End Point:

Part 1 SD:
Safety:
   Adverse Events (AEs) and Adverse Drug Reactions (ADRs)
   Vital signs: Systolic (SBP) and Diastolic (DBP) Blood Pressure
   12-lead ECG parameters extracted from Holter (HR, PR, QRS, QTcF, QT)
   Clinical laboratory evaluations (chemistry, haematology and urinalysis).
Part 2 MD:
Safety:
   AEs and ADRs;
   Vital signs: SBP, DBP; temperature;
   Triplicate 12-lead ECG;
(Should any ECG/cardiovascular findings emerge from Part 1 of the study, the SAC may also implement Holter monitoring for part, or all, of Part 2, as indicated);
   12-lead ECG parameters extracted from Holter (HR, PR, QRS, QTcF, QT);
   24 h Holter ECG abnormal findings (total pauses>2.5 secs, atrial fibrillation and atrial flutter, ventricular runs, premature atrial contractions (PAC) burden, premature ventricular contractions (PVC) burden, aberrant morphologies); 0-24 h heart rate (from 24 h Holter ECG) and hourly average HR;
   Clinical laboratory evaluations (chemistry, haematology and urinalysis).

Timepoint(s) of evaluation of this end point: indicated above.

### Secondary End Point:

### Part 1 SD:

### Pharmacokinetic Variables:

The following PK parameters will be derived from serum concentrations of the polypeptide of SEQ ID NO: 4:
AUC 0-12 h, AUC00-24 h, AUC0-t, AUC0-∞, Cmax, tmax, t½, CL/F, Vd/F;
AUC 0-12 h DN, AUC00-24 h DN, AUC0-t DN, AUC0-∞ DN, Cmax DN.

Immunogenicity variables: ADA Ct

### Part 2 MD:

### Pharmacokinetic Variables:

The following PK parameters will be derived from serum concentrations of the polypeptide of SEQ ID NO: 4:
On Day 1: AUC 0-12 h, Cmax, and tmax;
From Day 2 to Day 13: Ctrough
On the last day of drug administration (Day 14): AUC 0-12 h, AUC0-t, AUC0-∞, Ctrough, Cmax, Cmin, tmax, tmin, Cav, and Rac, t½, CL/F, and Vd/F.

### Immunogenicity Variables:

Antidrug antibody (ADA) serum concentrations will be evaluated on Day 1 prior to the first dose application, on Day 15 prior to discharge, on Day 24 (Week 4), Day 52 (Week 8) and on Day 80 (Week 12).
Ct

### Pharmacodynamic/Efficacy Variables:

Mean reduction in target ulcer area and volume from Baseline to D14, D21, D28, D56 and D84;
Time to healing of the target ulcer area and volume. Healing will be defined as “complete recovery”. Different “healing definitions will also be applied (Partial reduction: 50%, 66%, 75%).

Time point(s) of evaluation of this end point: indicated above.

The single ascending dose (SAD) is carried out at the following dosages: Cohort A: 0.3 μg/mm²; Cohort B: 1 μg/mm²; Cohort CA: 3 μg/mm²; Cohort D: 6 μg/mm². Each cohort is comprised of SEQ ID NO: 4-native subjects in order to avoid the possibility of carry-over effects between cohorts. This is particularly key with regard to hyperalgesia (known for wild-type human NGF. If necessary, dose levels are adjusted and washout periods may be included, if needed, to meet the study objectives.

In the single ascending dose (SAD), Standard of Care (SOC) was given at screening and at each consecutive visit until the end of the Follow-up Period, unless complete re-epithelialization/healing occurred, persistent for 2 consecutive visits. In this case, SoC could be discontinued and the subject's foot managed according to the evaluation/decision of the Investigator. The SoC consisted in the following procedures:
the debridement of the target ulcer (any possible bleeding caused by debridement were controlled only by compression and elevation of the leg),
the dressing of the lesion with paraffin gauze and covered with a protective bandaging made of sterile gauzes,
the use of an off-loading removable walker (unremovable during the Follow-Up Period) after bandaging.

In case of infection of the lesion during the study, the lesion had to be sampled for microbiological culture and the subject to be prescribed systemic empiric antibiotic therapy according to the decision of the investigator, who had to adjust the therapy according to the result of the culture. Every infection had to be evaluated and assessed for their seriousness, particularly when severe in intensity.

The multiple ascending dose (MAD) is carried out in two cohorts, each with native subjects, in sequence. Target daily dose levels are 1 μg/mm² and 3 μg/mm² with the polypeptide of SEQ ID NO: 4 (20) or placebo (10). After screening eligible subjects are treated according to standard of care (SOC; run-in period); enrolment confirmed after measuring the ulcer size. If during this run-in period the ulcer area reduced by 50% or more, the subjects will not be enrolled.

### Scope of the Trial:

Determination in humans of safety, pharmacokinetic, pharmacodynamics and others (tolerability) of the IMP.

### Results

### Single Ascending Dose (SOC)

The single ascending dose (SAD) was carried out in four consecutive cohorts at the following dosages: Cohort A: 0.3 [symbol] g/mm²; Cohort B: 1 [symbol] g/mm²; Cohort CA: 3 [symbol] g/mm²; Cohort D: 6 [symbol] g/mm², on top of the standard of care. In all those cohorts, quantifiable levels of the polypeptide of SEQ ID NO: 4 were detectable in the systemic blood circulation of the respective human subjects. Thus, the administered polypeptide is present in the body of the subjects after administration.

The subjects were monitored for adverse events for 28 days following administration of the single dose. No adverse events were linked to administration of the polypeptide of SEQ ID NO: 4. Thus, administration of the polypeptide of SEQ ID NO: 4 was not associated with any observable adverse drug reaction. As a result of the absence of adverse drug reactions, it is concluded that the polypeptide of SEQ ID NO: 4 is safe and well tolerated by human subjects.

### Outlook

The biological activity of the polypeptide of SEQ ID NO: 4 stems from its ability to promote growth, as well as the maintenance, proliferation and survival of cells, particularly nerve cells.

As a result of this example, the safety, tolerability, pharmacokinetic and pharmacodynamic profiles of the polypeptide of SEQ ID NO: 4 after single and repeated ascending doses in humans is investigated and confirmed.

### BRIEF DESCRIPTION OF THE FIGURES

FIG. 1: Outline of the process according to Example 2, including improvements described in Example 2B.

FIG. 2: IN VITRO PC12 NEURITE ELONGATION TEST.

Representation of neurite average length (A; average length in the well), neurite total length (B; average total length per cell) and percentage of cells showing a neurite longest than the cell body length (C).

Bars represent mean±SEM. Statistical analysis: One-way ANOVA followed by Tukey's post hoc versus the vehicle treated group (* P<0.05; ** P<0.01; *** P<0.001) CHF6467=polypeptide of SEQ ID NO: 4

FIG. 3. EFFICACY STUDY, 8 DAYS COHORT

Glucose blood levels in db/db mice measured at the time of skin biopsy. No differences were observed between the animals assigned to the different treatment groups.

CHF6467=polypeptide of SEQ ID NO: 4

FIG. 4. EFFICACY STUDY, 8 DAYS COHORT.

Body weight gain across the experimental time (day 0 and day 7 after the skin lesion).

CHF6467=polypeptide of SEQ ID NO: 4

FIG. 5: THE EFFICACY STUDY, 8 DAYS COHORT.

Thermal threshold. Latency to paw withdrawal at the plan tartest performed at −3 and 7 days after skin biopsy and NGF administration. Data are represented as mean+SEM. Statistical analysis performed by Student's t test, *p<0.05.

CHF6467=polypeptide of SEQ ID NO: 4

FIG. 6. THE EFFICACY STUDY, 8 DAYS COHORT.

“Time-to-closure” measured as external and internal area of the ulcer (see report from the pilot study for details) until day 8 from the skin biopsy. Results are expressed as % value of the lesioned area at day 0; data are represented as mean+SEM. See text for the two-ways ANOVA statistical analysis.

CHF6467=polypeptide of SEQ ID NO: 4

FIG. 7. B. THE EFFICACY STUDY, 8 DAYS.

Time-to-closure of the ulcer (external area) at day 8, expressed as the percentage of ulcer closure compared to day 0. Data are represented as mean+SEM.

CHF6467=polypeptide of SEQ ID NO: 4

FIG. 8: B. THE EFFICACY STUDY, 8 DAYS COHORT

NGF plasma levels at sacrifice Data are mean+SEM; Statistical analysis: one-way ANOVA followed by post-hoc Dunnett's test. * p<0.05, ** p<0.01.

CHF6467=polypeptide of SEQ ID NO: 4

FIG. 9. THE EFFICACY STUDY, 30 DAYS COHORT

Glucose blood concentration was measured at skin biopsy (d-1), after the end of treatment with the polypeptide of SEQ ID NO: 4 (d-8), and at sacrifice (d28). See text for details.

CHF6467=polypeptide of SEQ ID NO: 4

FIG. 10. THE EFFICACY STUDY, 30 DAYS COHORT

Body weight gain across the experimental time. No differences were observed among experimental groups.

CHF6467=polypeptide of SEQ ID NO: 4

FIG. 11: THE EFFICACY STUDY, 30 DAYS COHORT

Results from plantar test at 0 and 7 days after skin biopsy and administration of polypeptide of SEQ ID NO: 4 (N=8 in each group). Data obtained in the 30 days cohort are presented as mean+SEM; statistical analysis performed by Student's t test.

CHF6467=polypeptide of SEQ ID NO: 4

FIG. 12: THE EFFICACY STUDY, 30 DAYS COHORT

Thermal hyperalgesia measured at day 0 and at day 7 after the punch biopsy. In this graph data obtained from both 8 days and 30 days experiments are pooled (N=16 in each group); data are mean+SEM. Statistical analysis Student's t test, *p<0.05.

CHF6467=polypeptide of SEQ ID NO: 4

FIG. 13: THE EFFICACY STUDY, 30 DAYS COHORT.

The table lists the day of closure over the observational time, as derived from the clinical observation. “No” means that no closure was observed.

CHF6467=polypeptide of SEQ ID NO: 4

FIG. 14: THE EFFICACY STUDY, 30 DAYS COHORT.

Time course of the healing process measured as external area of the ulcer until day 29 from the skin biopsy. Results are expressed as % value of the lesion area at day 0; data are mean+SEM. See text for the statistical analysis.

CHF6467=polypeptide of SEQ ID NO: 4

FIG. 15: THE EFFICACY STUDY, 8+30 DAYS cohorts

Time-to-closure of the ulcer (external area) at day 8. Data depicted in the graph have been obtained by pooling values from both 8 days and 30 days experiments; N=16. Data are represented as mean+SEM. Statistical analysis: One-way ANOVA followed by post-hoc Dunnett's multiple comparisons test: F (5, 77)=3.007, P=0.0156.

CHF6467=polypeptide of SEQ ID NO: 4

FIG. 16: THE EFFICACY STUDY, 30 DAYS COHORT.

NGF plasma levels measured at day 30. Data are represented as mean+SEM; Statistical analysis: one-way ANOVA and post-hoc Dunnett's test.

CHF6467=polypeptide of SEQ ID NO: 4

FIG. 17: THE EFFICACY STUDY, 30 DAYS COHORT.

A. Micrographs illustrating the wound histology (H&E staining) at 7 days, as sampled according to the scheme present in B.

D-F. Microphotographs of the wound border illustrating the MET (epidermis migrating tongue, D,E) and angiogenesis (F).

FIG. 18: THE EFFICACY STUDY, 30 DAYS COHORT.

Representative micrographs of the re-epithelialization process taken from vehicle, SEQ ID NO: 4 (1, 10 and 30 μg/day)-treated mice. For comparative purposes, also a microphotograph of the intact skin is reported. The thickness of epidermal layer in the different groups is reported in the graph. Data are reported as mean+SEM; Statistical analysis: one-way ANOVA followed by post-hoc Tukey's test, **p<0.01; ****p<0.0001.

CHF6467=polypeptide of SEQ ID NO: 4

FIG. 19: THE EFFICACY STUDY, 30 DAYS COHORT.

Anatomy of the innervation of the mouse skin back, as visualized by PGP-9.5 immunostaining.

FIG. 20: THE EFFICACY STUDY, 8+30 DAYS COHORTS.

PGP9.5-IR in the skin of intact animals (A), vehicle (B) and SEQ ID NO: 4 (30 μg/day)-treated (C) mice, at 30 days after lesion induction. D, E: ROI (region of interest, D) and threshold setting (E) for the computerized image analysis procedure used to evaluate skin innervation at 30 days.

FIG. 21: THE EFFICACY STUDY, 8+30 DAYS COHORTS.

PGP9.5-IR morphometric analysis in the skin at 8 (A) and 30 (B) days after wound induction. Data are expressed as mean+SEM; Statistical analysis: one-way ANOVA followed by Dunnett's post-hoc test; *p<0.05; **p<0.01.

CHF6467=polypeptide of SEQ ID NO: 4

FIG. 22: THE EFFICACY STUDY, 8+30 DAYS COHORTS.

Histological (H&E staining) and immunohistochemical analysis of the repaired skin in the SEQ ID NO: 4 (30 μg/day)-treated group. A-B: repaired skin layers on a basal membrane, as indicated by the laminin-staining (B, arrows); the reinnnervation is coming from the subepidermal plexus and projects up through the basal membrane (C). The MET (D) is highly innervated at both 8 (E) and 30 (F) days after; lesion; angiogenesis is observed in MET, as visualized by H&E staining (G), laminin-IR at low (H) and high (I) magnification.

Abbreviations: ep, epidermal layer; MET, epidermis migrating tongue

FIG. 23: THE EFFICACY STUDY, 8+30 DAYS COHORTS.

Laminin-IR morphometric analysis in the skin at 8 (A) and 30 (B) days after wound induction. The grey horizontal bar in the B graph represents the laminin-IR in the intact skin. Data are expressed as mean+SEM; Statistical analysis: one-way ANOVA followed by Dunnett's post-hoc test; *p<0.05; ***p<0.001; ****p<0.0001.

FIG. 24: Polypeptide sequences. Asterisk (*)=position 61 in mature human NGF; cross (+): position 100 in mature human NGF.

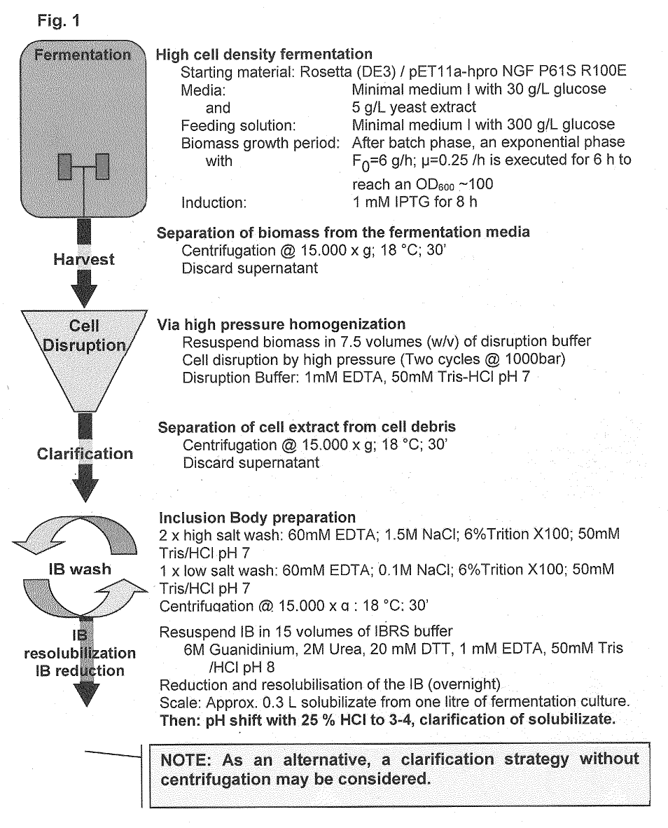

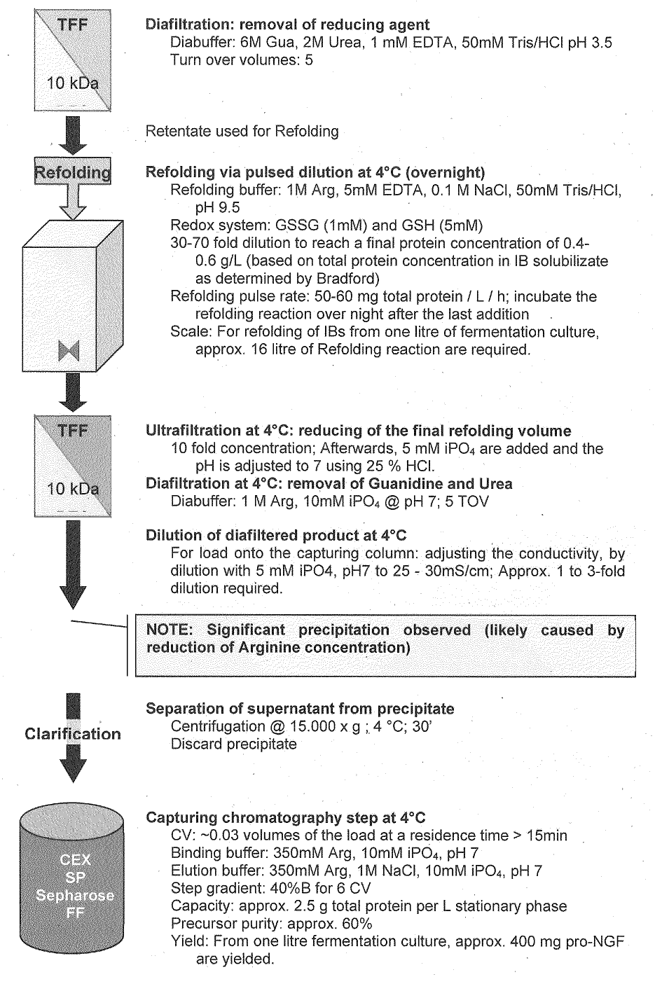

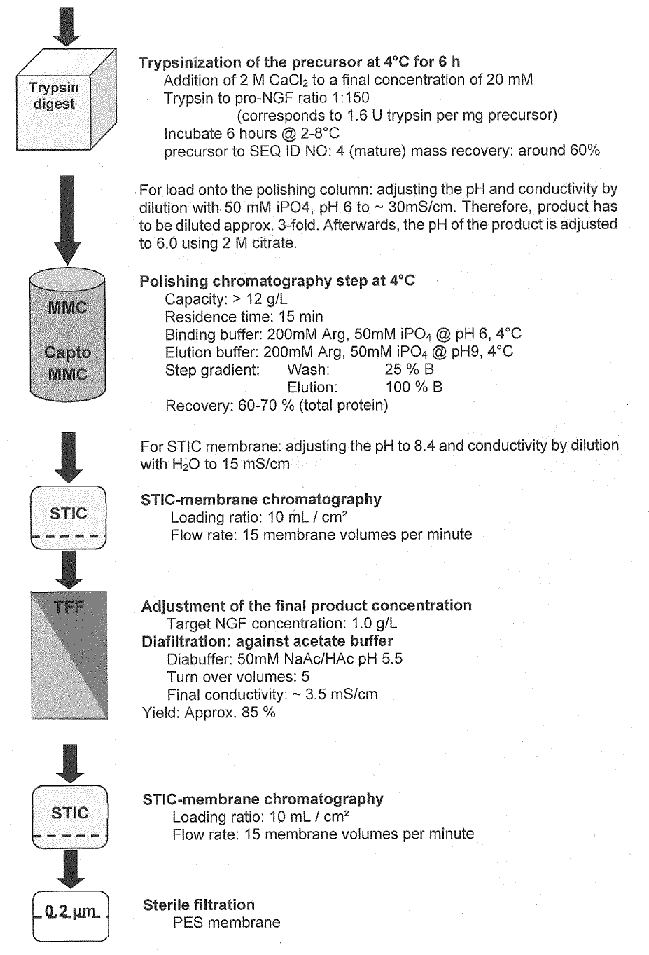

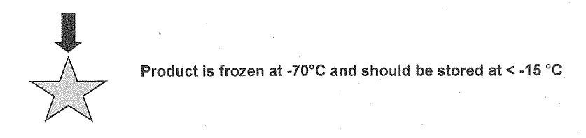

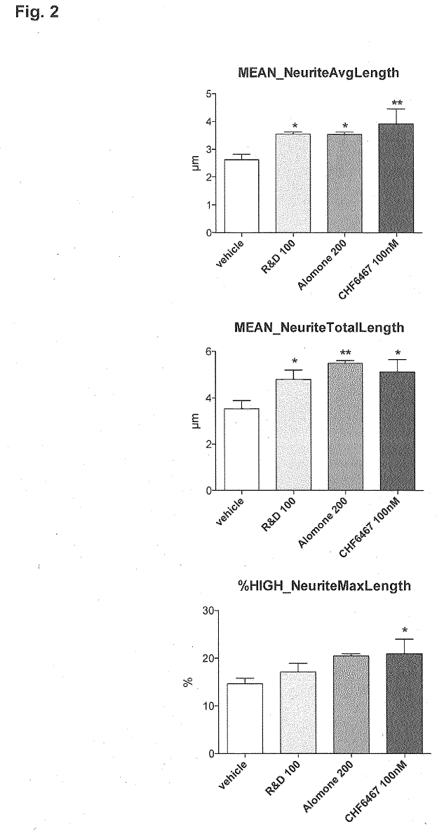

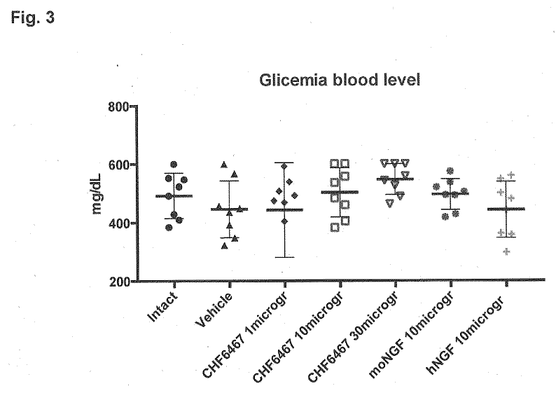

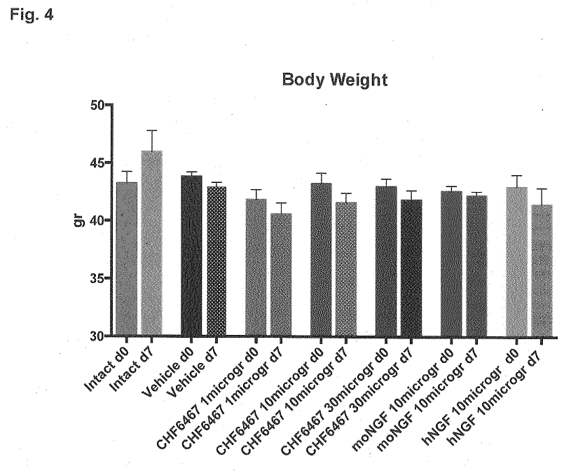

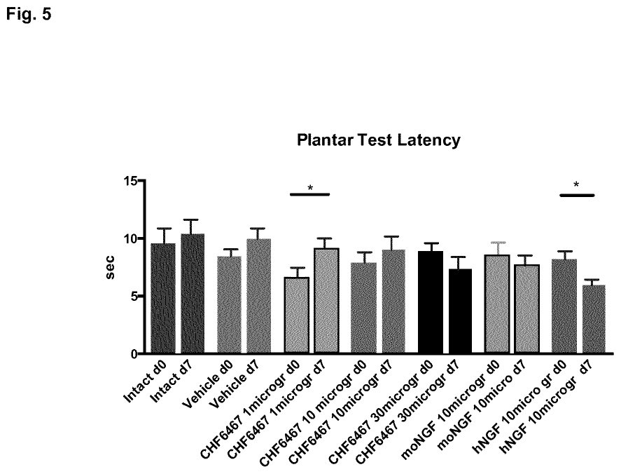

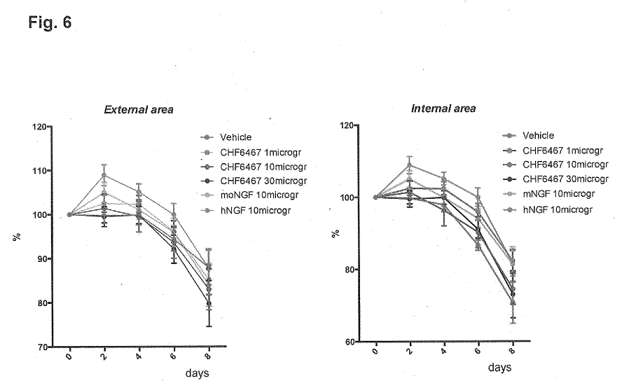

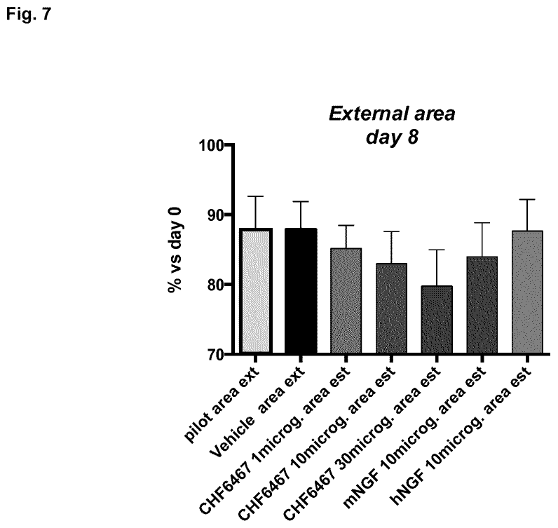

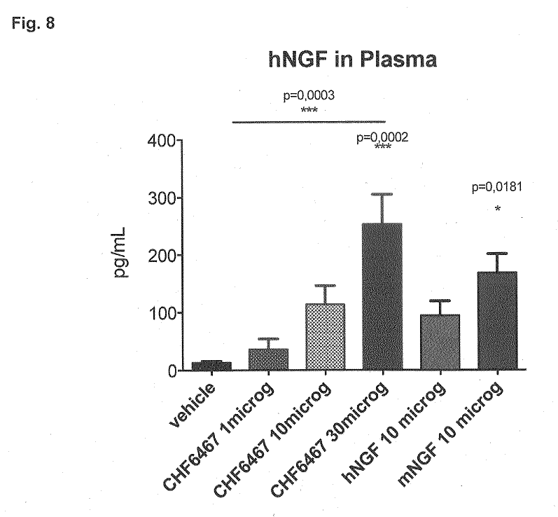

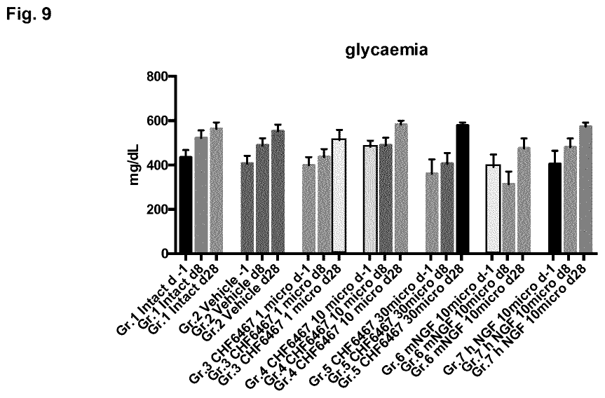

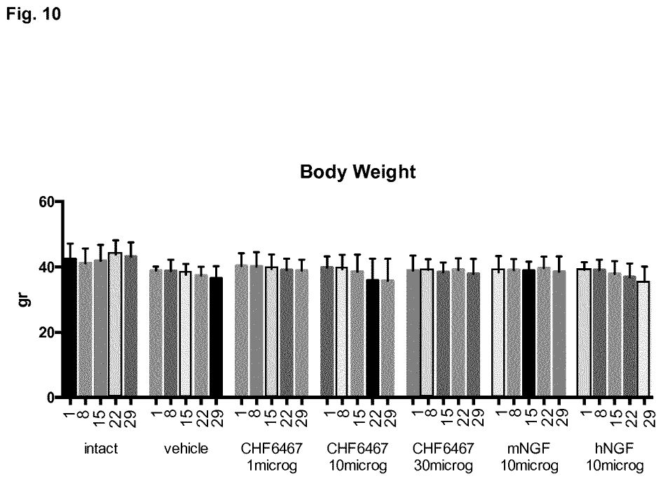

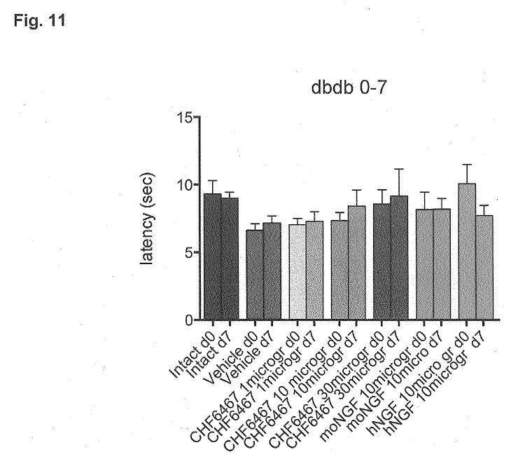

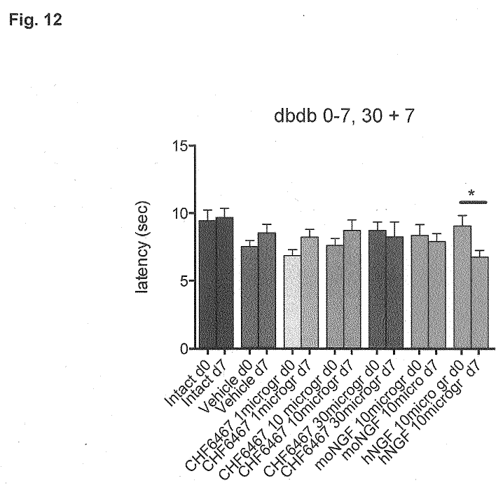

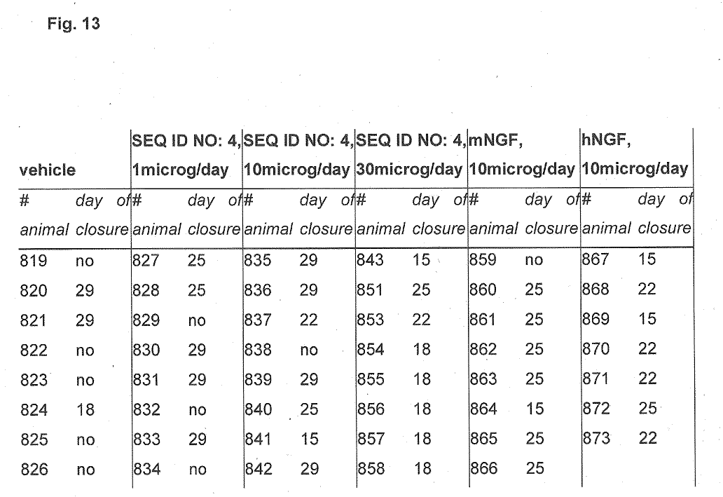

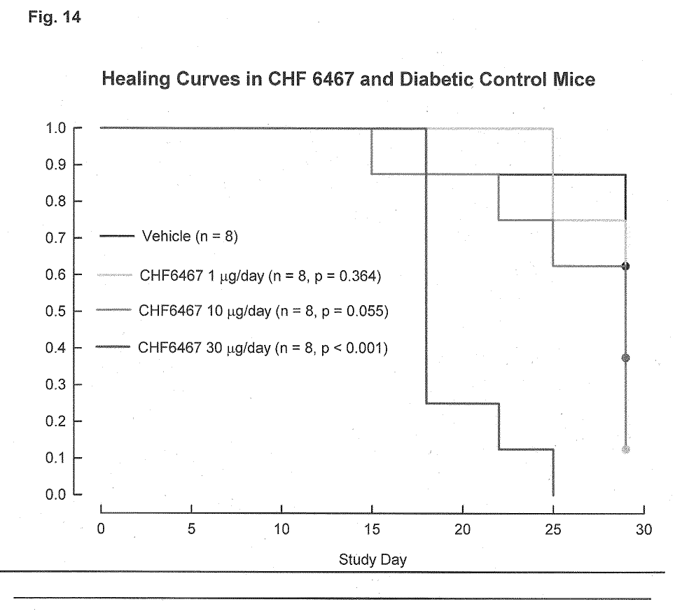

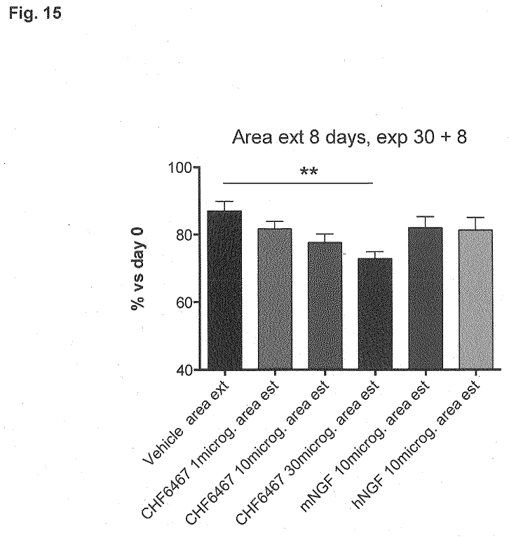

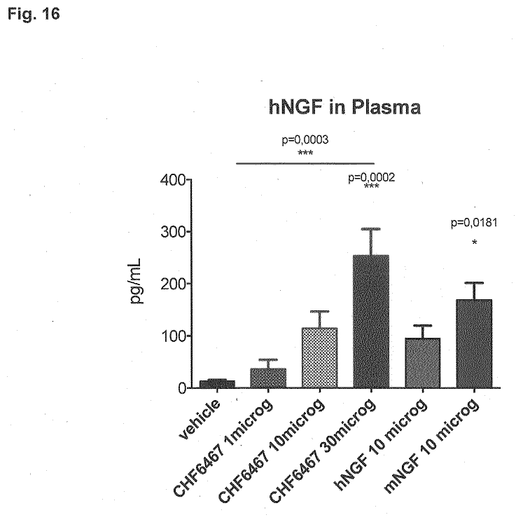

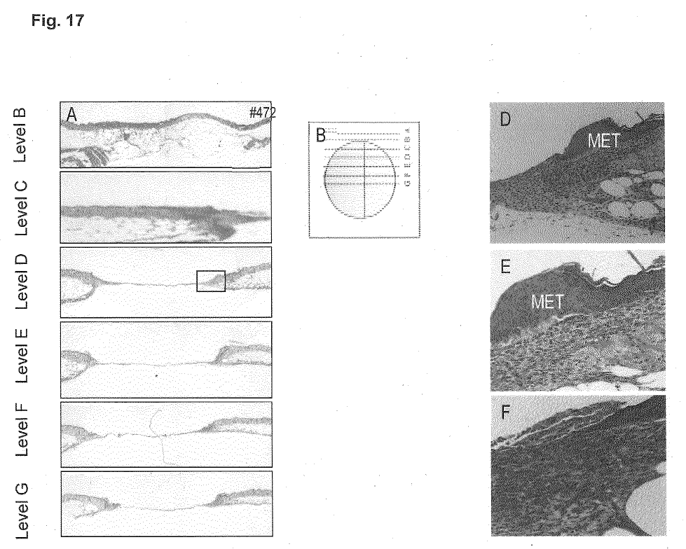

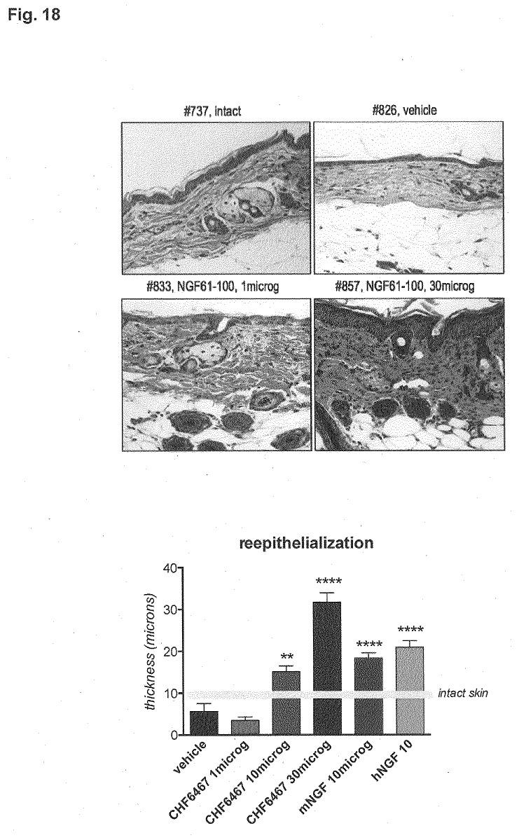

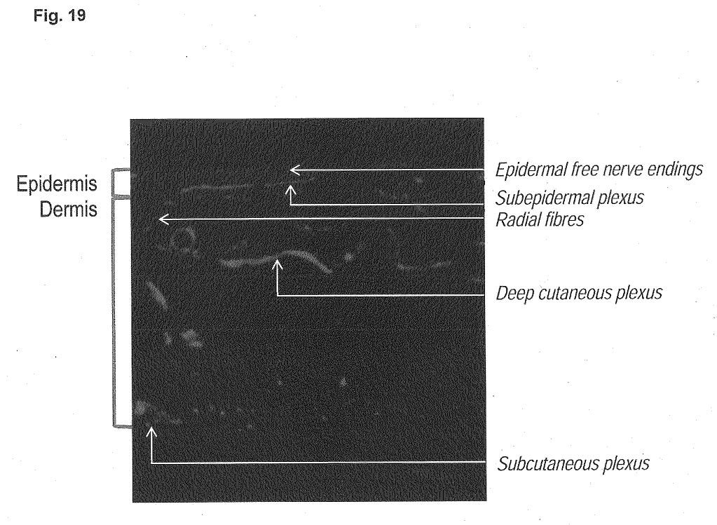

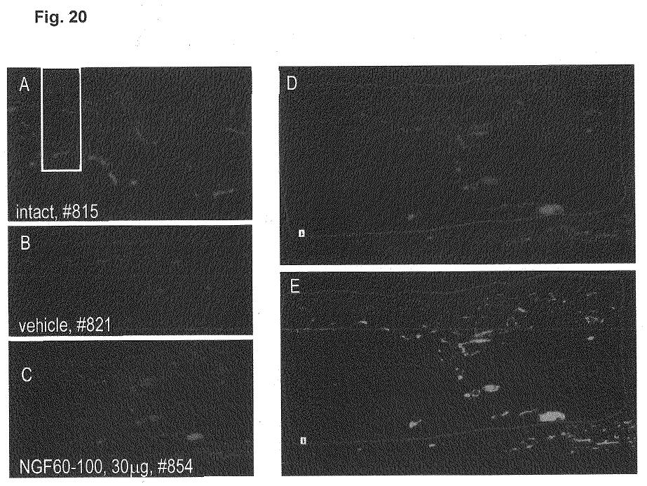

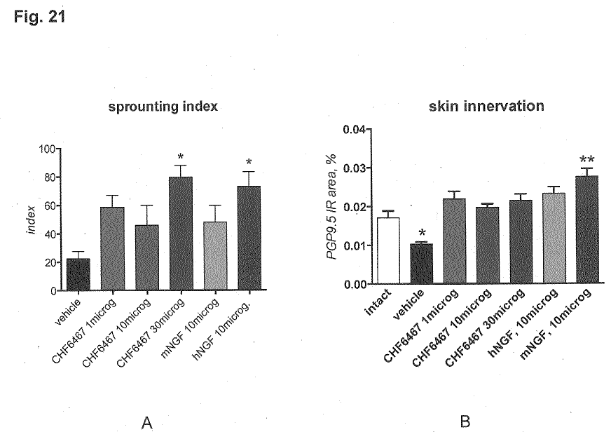

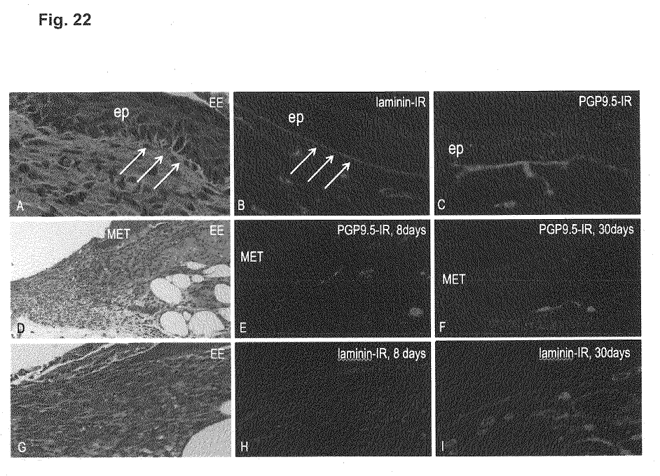

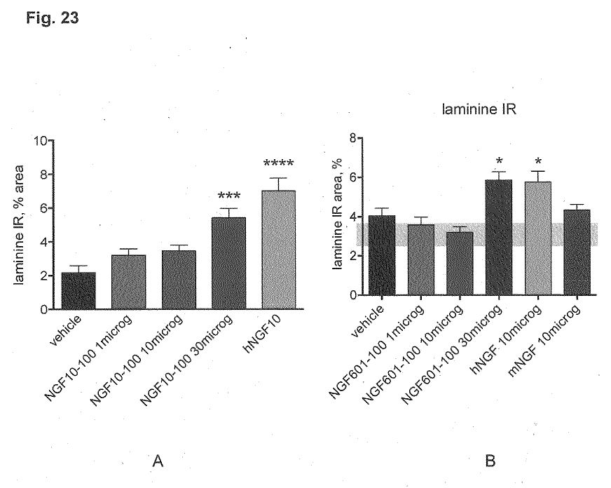

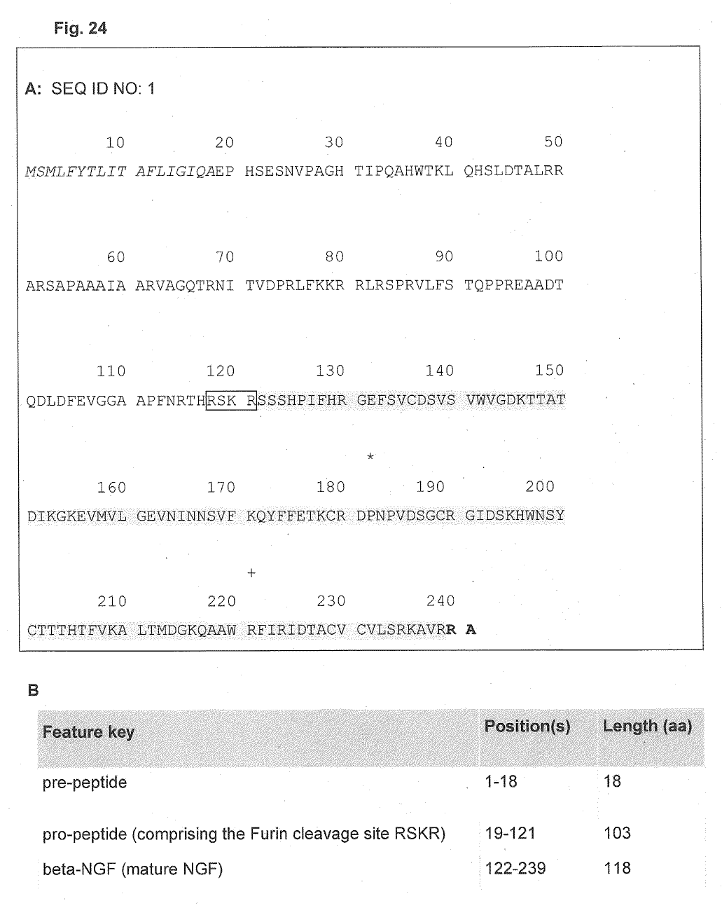

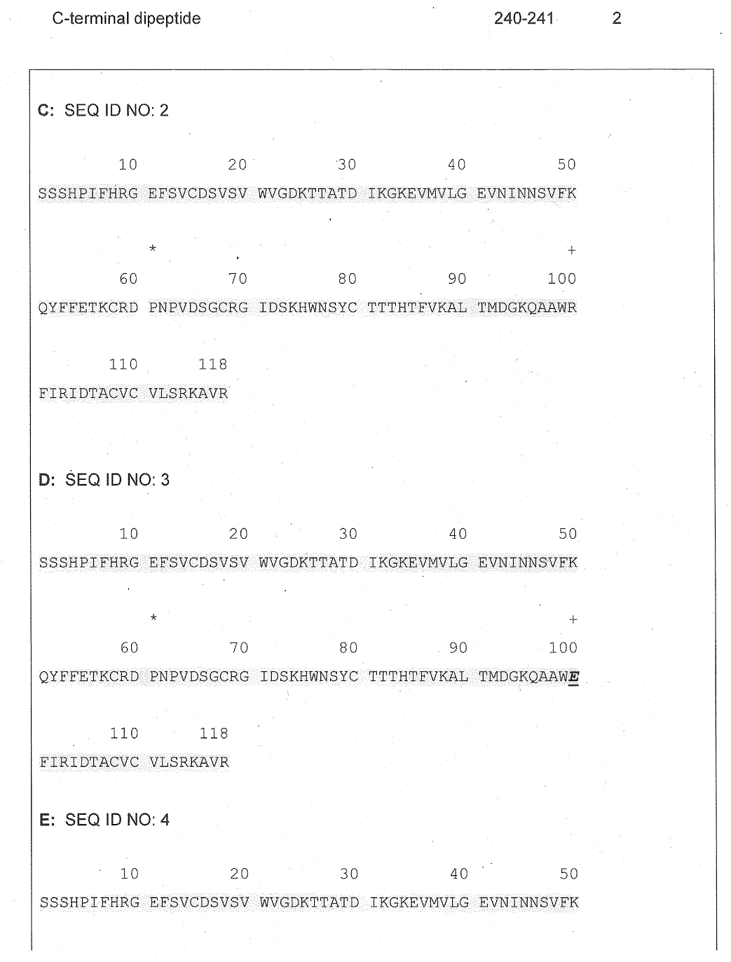

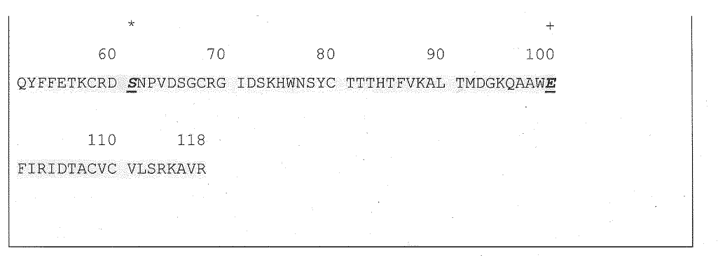

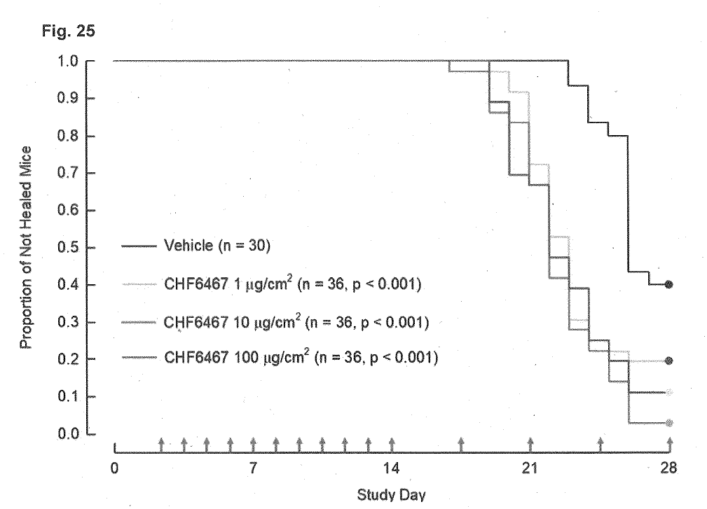

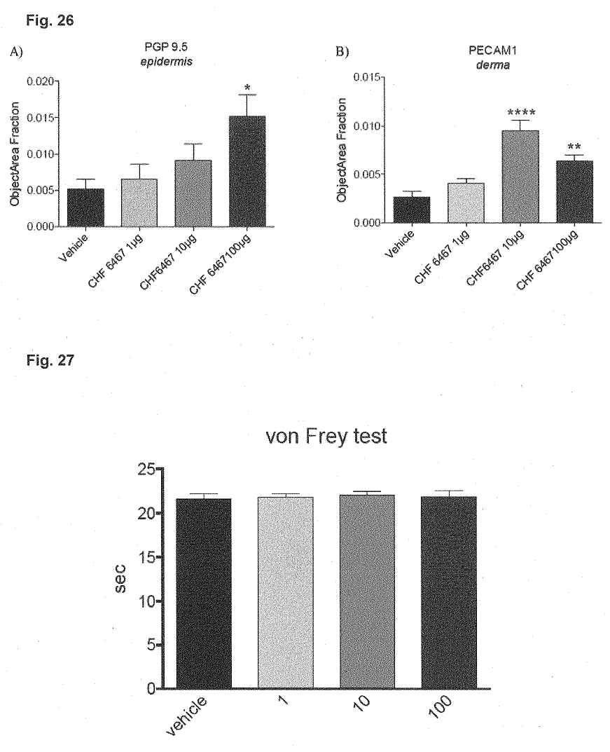

A: SEQ ID NO: 1: Sequence of pre-pro human NGF as encoded by the respective human Open Reading Frame.

Pre-peptide: amino acid positions 1-18; pro-peptide: amino acid positions 19-121; mature NGF: amino acid positions 122-239; C-terminal dipeptide: amino acid positions 240-241.

Disulfide bonds (in the correctly folded mature part): linking amino acid positions 136↔201, 179↔229, 189↔231.

Furin cleavage site (RSKR): amino acid positions 118-121.

B: Schematic overview of pre-peptide, pro-peptide and mature NGF.

C: SEQ ID NO: 2: Sequence of mature human NGF.

D: SEQ ID NO: 3

E: SEQ ID NO: 4

FIG. 25: The rate of wound healing after treatment with vehicle or polypeptide of SEQ ID NO: 4 (1-10-100 μg/cm²/day) in diabetic mice is represented as “Proportion of Not Healed Mice” over the entire course of the study. Statistical analysis: Kaplan-Meier survival curve estimate.

CHF6467=polypeptide of SEQ ID NO: 4

FIG. 26: PGP9.5-IR (A) and PECAM1-IR (A) morphometric analysis in the repaired skin at 28 days after wound induction. Data are expressed as mean±SEM (n=15-18). Statistical analysis: one-way ANOVA followed by Dunnett's post-hoc test; *p<0.05; **p<0.01.

CHF6467=polypeptide of SEQ ID NO: 4

FIG. 27: The effect of the polypeptide of SEQ ID NO: 4 on pain mechanical threshold in the area surrounding the ulcers border was evaluated after 14 days of chronic topical treatment with the Bioseb's electronic von Frey apparatus. Data are expressed as mean±SEM (n=15-18). Statistical analysis: one-way ANOVA.

1. A polypeptide selected among the polypeptide of SEQ ID NO: 3 and the polypeptide of SEQ ID NO: 4 for the use in treatment and/or prevention of a dermatological disorder in a mammalian subject.

2. The polypeptide for use according to claim 1, wherein the mammalian subject is a human.

3. The polypeptide for use according to any one of the preceding claims, wherein the polypeptide is the polypeptide of SEQ ID NO: 4.

4. The polypeptide for use according to any one of the preceding claims, wherein the dermatological disorder is characterized by wounded surface on at least a part of the body of the subject.

5. The polypeptide for use according to claim 4, wherein the dermatological disorder characterized by wounded surface is a skin lesion, preferably characterized by at least partial ablation of the dermis, and optionally of the dermis.

6. The polypeptide for use according to any one of the preceding claims, wherein the dermatological disorder comprises at least one ulcer, preferably selected from the group consisting of diabetic ulcers, trauma ulcers, surgical ulcers, pressure ulcers, chronic ulcers, and combinations of any of these ulcers, or wherein the dermatological disorder comprises a burn or mechanical injury.

7. The polypeptide for use according to any one of the preceding claims, wherein the mammal, preferably the human, suffers from diabetes mellitus or has a predisposition to suffer from diabetes mellitus.

8. The polypeptide for use according to claim 7, wherein the diabetes mellitus is selected among diabetes mellitus Type 1 and diabetes mellitus Type 2.

9. The polypeptide for use according to any one of the preceding claims, wherein the polypeptide is administered in a single administration.

10. The polypeptide for use according to any one of claims 1 to 8, wherein the polypeptide is administered repeatedly.

11. The polypeptide for use according to claim 10, wherein the polypeptide is administered repeatedly one to five times per day, preferably about twice per day.

12. The polypeptide for use according to claim 10 or claim 11, wherein the polypeptide is administered repeatedly, for a period of three to 30 days, preferably seven to 14 days, or alternatively until closure of the wounded body surface.

13. The polypeptide for use according to any one of the preceding claims, wherein the polypeptide is administered to subjects with diabetic foot ulcers (DFU), preferably to subjects with diabetic neuropathic foot ulcers (DFU).

14. The polypeptide for use according to any one of the preceding claims for topical administration.

15. The polypeptide for use according to claim 14, wherein the polypeptide is administered onto the wounded body surface.

16. The polypeptide for use according to claim 15, wherein the dose/each dose has an amount of 0.3 to 6 μg of the polypeptide per mm² of wounded body surface being treated (0.3 to 6 μg/mm²).

17. The polypeptide for use according to any one of the preceding claims, wherein the treatment and/or prevention does not cause hyperalgesia in the mammalian subject.

18. The polypeptide for use according to any one of the preceding claims, wherein the polypeptide is comprised in an aqueous medium, and the aqueous medium is administered to the mammalian subject.

19. The polypeptide for use according to any one of the preceding claims, wherein the polypeptide is obtainable by recombinant expression and purification, wherein the purification comprises purification on a mixed mode stationary phase.
